SEQUENCE LISTING (74 sequences)

SEQ ID NO: 1 - DNA, 5 bp, Artificial Sequence
  HBS
rcgts

SEQ ID NO: 2 - DNA, 5 bp, Artificial Sequence
  has
casrb

SEQ ID NO: 3 - DNA, 6 bp, Artificial Sequence
  hnf4
tgacct

SEQ ID NO: 4 - DNA, 6 bp, Artificial Sequence
  linker
gtctca

SEQ ID NO: 5 - DNA, 26 bp, Homo sapiens
gggccctacgtgctgtctcacacagc

SEQ ID NO: 6 - DNA, 26 bp, Mus musculus
gggccctacgtgctgcctcgcatggc

SEQ ID NO: 7 - DNA, 25 bp, Homo sapiens
tgtcacgtcctgcacgacgcgagta

SEQ ID NO: 8 - DNA, 24 bp, Mus musculus
cgcgtcgtgcaggacgtgacaaat

SEQ ID NO: 9 - DNA, 30 bp, Mus musculus
ccagcggacgtgcgggaacccacgtgtagg

SEQ ID NO: 10 - DNA, 26 bp, Homo sapiens
tccacaggcgtgccgtctgacacgca

SEQ ID NO: 11 - DNA, 35 bp, Homo sapiens
ccacagtgcatacgtgggctccaacaggtcctctt

SEQ ID NO: 12 - DNA, 8 bp, Homo sapiens
tacgtggg

SEQ ID NO: 13 - DNA, 24 bp, Rattus exulans
acagtgcatacgtgggcttccaca

SEQ ID NO: 14 - DNA, 17 bp, Homo sapiens
actacgtgctgcctagg

SEQ ID NO: 15 - DNA, 26 bp, Homo sapiens
cccctcggacgtgactcggaccacat

SEQ ID NO: 16 - DNA, 37 bp, Homo sapiens
acgctgagtgcgtgcgggactcggagtacgtgacgga

SEQ ID NO: 17 - DNA, 26 bp, Mus musculus
cggacgctggcgtggcacgtcctctc

SEQ ID NO: 18 - DNA, 470 bp, Moloney murine sarcoma virus
ctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 19 - DNA, 606 bp, Moloney murine sarcoma virus
ctagcggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgactctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 20 - DNA, 145 bp, Moloney murine sarcoma virus
gaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgg

SEQ ID NO: 21 - DNA, 107 bp, Moloney murine sarcoma virus
ctagagaaccatcagatgtttccagggtgccccaaggacctgaaatgaccctgtgccttatttgaactaaccaatcagttcgcttctcgcttctgttcgcgcgcttc

SEQ ID NO: 22 - DNA, 60 bp, Moloney murine sarcoma virus
tgctccccgagctcaataaaagagcccacaacccctcactcggggcgccagtcctccgat

SEQ ID NO: 23 - DNA, 52 bp, Moloney murine sarcoma virus
tgactgagtcgcccgggtacccgtgtatccaataaaccctcttgcagttgca

SEQ ID NO: 24 - DNA, 143 bp, Moloney murine sarcoma virus
gcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 25 - DNA, 11 bp, Moloney murine sarcoma virus
ggggtctttca

SEQ ID NO: 26 - DNA, 255 bp, Homo sapiens
ggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtctcacacagcctgtctgacggccctacgtgctgtct

SEQ ID NO: 27 - DNA, 34 bp, Homo sapiens
ggccctacgtgctgtctcacacagcctgtctgac

SEQ ID NO: 28 - protein, 18 aa, Homo sapiens
  MISC_FEATURE (1)..(1): can be any amino acid
  MISC_FEATURE (2)..(2): can be any amino acid
  MISC_FEATURE (11)..(11): can be any amino acid
  MISC_FEATURE (16)..(16): can be any amino acid
  MISC_FEATURE (17)..(17): can be any amino acid
  MISC_FEATURE (18)..(18): can be any amino acid
XXLEMLAPYIXMDDDXXX

SEQ ID NO: 29 - protein, 203 aa, Homo sapiens
APAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQ

SEQ ID NO: 30 - protein, 74 aa, Homo sapiens
EFKLELVEKLFAEDTEAKNPFSTQDTDLDLEMLAPYIPMDDDFQLRSFDQLSPLESSSASPESASPQSTVTVFQ

SEQ ID NO: 31 - protein, 124 aa, Homo sapiens
EFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTS

SEQ ID NO: 32 - protein, 55 aa, Homo sapiens
VVSHFNDCPLSHDGYCLHDGVCMYIEALDKYACNCVVGYIGERCQYRDLKWWELR

SEQ ID NO: 33 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 35 - protein, 500 aa, Artificial Sequence
  fusion
MGPGVLLLLLVATAWHGQGGVVSHFNDCPLSHDGYCLHDGVCMYIEALDKYACNCVVGYIGERCQYRDLKWWELRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQ

SEQ ID NO: 36 - DNA, 1500 bp, Artificial Sequence
  fusion
atgggcccaggagttctgctgctcctgctggtggccacagcttggcatggtcagggaggtgtggtgtcgcacttcaatgactgtccactgtcgcacgatggatactgcctccatgatggtgtgtgcatgtacatcgaggcattggacaagtatgcatgcaactgtgtcgtcggctacatcggagagcgatgtcagtaccgagacctgaagtggtgggaactgagagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccag

SEQ ID NO: 37 - protein, 13 aa, Homo sapiens
LEMLAPYIPMDDD

SEQ ID NO: 38 - DNA, 31 bp, Artificial Sequence
  primer
tccagcggctggggcgcgagggggcagggcc

SEQ ID NO: 39 - DNA, 31 bp, Artificial Sequence
  primer
ggccctgccccctcgcgccccagccgctgga

SEQ ID NO: 40 - DNA, 24 bp, Artificial Sequence
  primer
ccatggtgaagcgtgagaaaaatg

SEQ ID NO: 41 - DNA, 29 bp, Artificial Sequence
  primer
ctcgagttacttgtacagctcgtccatgc

SEQ ID NO: 42 - DNA, 30 bp, Artificial Sequence
  primer
gagaaggccggcggtgccccagccgctgga

SEQ ID NO: 43 - DNA, 50 bp, Artificial Sequence
  primer
cctcaaagcacagttacagtattccagggaagcggagctactaacttcag

SEQ ID NO: 44 - DNA, 24 bp, Artificial Sequence
  primer
ccatggtgaagcgtgagaaaaatg

SEQ ID NO: 45 - DNA, 29 bp, Artificial Sequence
  primer
ctcgagttacttgtacagctcgtccatgc

SEQ ID NO: 46 - DNA, 22 bp, Artificial Sequence
  primer
tgtctgtcgcagtaagaatgga

SEQ ID NO: 47 - DNA, 20 bp, Artificial Sequence
  primer
aaaatcccagacacggtggg

SEQ ID NO: 48 - DNA, 21 bp, Artificial Sequence
  primer
tttggtgtttgcttcagtcag

SEQ ID NO: 49 - DNA, 25 bp, Artificial Sequence
  primer
atacctagaaaacaggagttgctca

SEQ ID NO: 50 - DNA, 20 bp, Artificial Sequence
  primer
atttgactgccggcgaaatg

SEQ ID NO: 51 - DNA, 21 bp, Artificial Sequence
  primer
aagattcatcgccgaccacat

SEQ ID NO: 52 - DNA, 15 bp, Artificial Sequence
  primer
cggagaaagcgcagc

SEQ ID NO: 53 - DNA, 17 bp, Artificial Sequence
  primer
gggtccggggttctctt

SEQ ID NO: 54 - DNA, 36 bp, Artificial Sequence
  primer
ccacctgtaggtttggcaagctagcgtccgggaaac

SEQ ID NO: 55 - DNA, 38 bp, Artificial Sequence
  primer
ccacctgtaggtttggcaagctagccacgcctgtaatc

SEQ ID NO: 56 - DNA, 36 bp, Artificial Sequence
  primer
ccacctgtaggtttggcaagctagcccccctttggg

SEQ ID NO: 57 - DNA, 21 bp, Artificial Sequence
  primer
gaaccatcagatgtttccagg

SEQ ID NO: 58 - DNA, 18 bp, Artificial Sequence
  primer
atccgccacaacatcgag

SEQ ID NO: 59 - DNA, 39 bp, Artificial Sequence
  primer
cctggaaacatctgatggttctctagacctcaggcccgg

SEQ ID NO: 60 - DNA, 19 bp, Artificial Sequence
  primer
gcgggcctcttcgctatta

SEQ ID NO: 61 - DNA, 19 bp, Artificial Sequence
  primer
ttgccaaacctacaggtgg

SEQ ID NO: 62 - DNA, 33 bp, Artificial Sequence
  primer
ggtggtaccggtctgtaggtttggcaagctagc

SEQ ID NO: 63 - DNA, 22 bp, Artificial Sequence
  primer
gaaagaccccacctgtaggttt

SEQ ID NO: 64 - DNA, 38 bp, Artificial Sequence
  primer
gccacgaccggtgccgccaccatcccctgacccacgcc

SEQ ID NO: 65 - DNA, 30 bp, Artificial Sequence
  primer
gggtatataatggaagctcgaattctagcg

SEQ ID NO: 66 - DNA, 38 bp, Artificial Sequence
  primer
cgaaaggagcgcacgaccaattcaattggccctacgtg

SEQ ID NO: 67 - DNA, 15 bp, Artificial Sequence
  primer
cggatggccgcgaga

SEQ ID NO: 68 - DNA, 20 bp, Artificial Sequence
  primer
atttgactgccggcgaaatg

SEQ ID NO: 69 - DNA, 21 bp, Artificial Sequence
  primer
aagattcatcgccgaccacat

SEQ ID NO: 70 - DNA, 45 bp, Artificial Sequence
  primer
cctggaaacatctgatggttctctagatttggccatgttgactag

SEQ ID NO: 71 - DNA, 45 bp, Artificial Sequence
  primer
cctggaaacatctgatggttctctagagttccggggttagtcagt

SEQ ID NO: 72 - DNA, 22 bp, Artificial Sequence
  primer
caccaaagagtcctaaacgatc

SEQ ID NO: 73 - DNA, 38 bp, Artificial Sequence
  primer
cacgtagggccaattgaattggtcgtgcgctcctttcg

SEQ ID NO: 74 - protein, 18 aa, Homo sapiens
LDLEMLAPYIPMDDDFQL

(19) United States
(12) Patent Application Publication
(10) Pub. No.: US 2022/0195009 A1
(43) Pub. Date: Jun. 23, 2022

(54) HYPOXIA-RESPONSIVE CHIMERIC ANTIGEN RECEPTORS

(71) Applicant: KING'S COLLEGE LONDON, Strand, London (GB)

(72) Inventors: James Noble ARNOLD, Strand, London (GB); John MAHER, Strand, London (GB); Paraskevas KOSTI, Strand, London (GB)

(21) Appl. No.: 17/431,859

(22) PCT Filed: Feb. 19, 2020
(86) PCT No.: PCT/GB2020/050401

§ 371 Date: Aug. 18, 2021

(30) Foreign Application Priority Data
Feb. 19, 2019 (GB) 1902277.1

(51) Int. Cl. C07K 14/705; C12N 15/62; C12N 5/0783; A61P 35/00; A61K 35/17; C07K 14/725

(52) U.S. Cl. CPC C07K 14/70567; C12N 15/62; C12N 5/0636; A61P 35/00; A61K 35/17; A61K 2039/5156; C07K 2319/30; C07K 2319/33; C12N 2510/00; C12N 2500/02; C07K 14/7051

(57) ABSTRACT

The present invention relates to therapeutic agents, particularly to therapeutic polypeptides and nucleic acids having the capacity for selective expression under conditions of hypoxia, cells incorporating the nucleic acids and their use in therapy, in particular in methods requiring selective expression under conditions of hypoxia, such as typically found in solid cancers. The nucleic acids encode novel hypoxia-responsive chimeric antigen receptors (CARs). The invention also relates to hypoxia-responsive regulatory nucleic acids.

### CROSS-REFERENCE TO RELATED APPLICATIONS

This application is a U.S. National Stage Application of International Application No. PCT/GB2020/050401, filed Feb. 19, 2020, which claims the benefit of, and priority to, GB Patent Application No. 1902277.1, filed Feb. 19, 2019, the entire contents of which are hereby incorporated by reference in their entirety.

### INCORPORATION BY REFERENCE OF SEQUENCE LISTING

This application contains a Sequence Listing that has been submitted in ASCII format via EFS-Web and is hereby incorporated by reference in its entirety. The ASCII copy, created on Feb. 19, 2019, is named P3468PC00 Sequence Listing_ST25.txt, and is 1800 bytes in size. A corrected listing created Sep. 16, 2021, is named P3468US00_Corrected_Sequence_Listing.txt, and is 26,000 bytes in size.

### TECHNICAL FIELD

The present invention relates to therapeutic agents, particularly to therapeutic polypeptides and nucleic acids capable of hypoxia-responsive expression, cells incorporating the same and their use in therapeutic or prophylactic treatment, in particular in methods requiring selective expression of the therapeutic agent under conditions of hypoxia, such as typically found in a solid cancer environment. The nucleic acids may encode novel hypoxia-responsive chimeric antigen receptors (CARs). The invention also relates to hypoxia-responsive regulatory nucleic acids.

### BACKGROUND

T-cells engineered to express chimeric antigen receptors (CARs) or engineered T-cell receptors (TCRs) are an effective way of re-directing the immune system to target and destroy cancer cells in the human body. CAR T-cell (CAR-T) therapy in particular has shown great promise as an effective and viable treatment for haematological cancers. However, the complexity of the solid cancer microenvironment poses a challenge to the current CAR-T approaches. One main hurdle is the paucity of tumour-specific target antigens, the absence of which can result in off-target CAR T-cell activation within normal tissues with consequent side-effects. Upon antigen binding, CARs initiate robust T-cell activation and subsequent cytolytic killing of the target cell. However, the selectivity of CAR-mediated killing of the tumour cells is currently dictated solely by the biodistribution of the CAR antigen. In current approaches, tumour selectivity is therefore crucial to the success of CAR-T therapy as on-target off-tumour activation of CAR T-cells can result in potentially lethal toxicities.

Hypoxia is characteristic of most solid tumours, where proliferative and high metabolic demands of the tumour cells, alongside inefficient tumour vasculature, result in a state of inadequate oxygen supply (<2% O₂) compared to that of healthy organs/tissues (5-10% O₂). Clinically, hypoxia has been associated with poor prognosis, and resistance to both chemotherapy and radiotherapy. Cells have evolved an elegant biological machinery to both detect and rapidly respond to hypoxia through the constitutively expressed transcription factor hypoxia-inducible factor alpha (HIF1α). Under conditions of sufficient O₂, HIF1α is degraded through hydroxylation of two prolines in an Oxygen-Dependent Degradation Domain (ODD) within its structure. Hydroxylated ODDs are subsequently recognised by von Hippel-Lindau tumour suppressor, which forms part of an E3 ubiquitin ligase complex, that ubiquitinates HIF1α and thereby targets it for proteasomal degradation. Conversely, under limiting O₂ concentrations HIF1α becomes stabilised and translocates to the nucleus where it binds to HIF1β and p300/CBP. This complex can then associate with Hypoxia Responsive Elements (HREs) in the promoter region of several hypoxia-responsive genes initiating transcription.

Various cancer therapies that exploit low oxygen tension are in development, including amongst others hypoxia-specific gene therapy, hypoxia-activated pro-drugs, HIF1-interacting drugs and obligate anaerobic bacteria. As hypoxia differentiates the tumour microenvironment from that of healthy, normoxic tissue, it represents a desirable marker for the induction of CAR T-cell expression. Juillerat et al., 2017, (Scientific Reports 7, 39833) investigated CARs fused with an ODD. Although this approach endowed CAR T-cells with an improved ability to kill tumour cells under hypoxic conditions in vitro, the authors observed residual tumour killing under normoxic conditions, indicating undesirable leakiness of the system.

It would be desirable to develop therapeutic nucleic acids, polypeptides, and engineered cells, for example in the form of a CAR and CAR T-cells, capable of stringently restricting expression to areas of hypoxia so as to reduce off-target effects. This in turn would allow treatment, particularly of solid cancers, to be extended to a wider variety of tumour antigens, particularly to those found on normal tissues as well as on tumours.

It would also be desirable to improve the enabling technologies for driving and regulating expression of the therapeutic agents at the tumour site.

It would also be desirable to be able to determine, prior to treatment, a subject's suitability for CAR T-cell therapy.

### SUMMARY OF THE INVENTION

The applicants have devised a dual oxygen-sensing system comprising a nucleic acid molecule encoding a chimeric polypeptide comprising one or more Oxygen dependent Degradation Domains (ODD) and at least one polypeptide with anti-tumour properties, which nucleic acid molecule is operably linked to a hypoxia-responsive regulatory nucleic acid comprising, consisting essentially of, or consisting of a plurality of Hypoxia Responsive Elements (HREs). This allows the nucleic acid molecule to be expressed under hypoxic conditions but with negligible expression under normoxic conditions. The dual oxygen-sensing system further provides for degradation, in normoxic conditions, of at least one polypeptide with anti-tumour properties, owing to the presence of the ODD, in combination with the action of the hypoxia-responsive regulatory nucleic acid. The nucleic acid molecule and/or chimeric polypeptide may be comprised in and/or expressed in a chimeric antigen receptor (CAR) and/or in immunoresponsive cells, for example. The combined use of a CAR-linked to one or more ODD and expressed under the control of a hypoxia-responsive regulatory nucleic acid is referred to herein as “hypoxiCAR”. The combined use of the hypoxia-responsive regulatory nucleic acid, which allows for expression only in substantially hypoxic conditions, along with the capability conferred by the one or more ODDs to cause degradation in normoxic conditions of the polypeptide with anti-tumour properties, allows for a reduction or substantial elimination of any off-target effects.

The applicants have also developed methods for determining a subject's suitability for treatment with a hypoxiCAR. This may be done by monitoring for the co-expression of any two, three, four or five the following genes: PGK1, SLC2A1, CA9, ALDOA and VEGFA, wherein such co-expression is indicative of the subject's suitability for treatment. Alternatively or additionally, a tumour biopsy from a subject may be immunohistochemically stained and assessed for HIF stabilisation in the tumour or stroma and/or for infiltration of T cells or other immunoresponsive cells to HIF stabilised regions of the tumour, wherein such HIF stabilisation or infiltration of the immunoresponsive cells to HIF stabilised regions of the tumour is indicative of a subject's suitability for treatment.

The applicants have also discovered that the hypoxia-responsive regulatory nucleic acids of the invention are better able to drive and regulate expression at the site of a solid tumour relative to conventional regulatory nucleic acids.

### DETAILED DESCRIPTION

Hypoxia-Responsive Regulatory Nucleic Acid

A first aspect of the present invention provides a hypoxia-responsive regulatory nucleic acid comprising, consisting essentially of, or consisting of a plurality of hypoxia-responsive elements (HREs). The hypoxia-responsive regulatory nucleic acid is capable of driving and regulating expression of a nucleic acid molecule preferentially under conditions of hypoxia.

The hypoxia-responsive regulatory nucleic acid may be derived from or based on a known regulatory nucleic acid modified to introduce therein a plurality of HREs. Alternatively, the plurality of HREs alone may themselves have regulatory function, i.e. the capability to initiate transcription and to drive expression of a nucleic acid molecule operably linked thereto. In such cases the plurality of HREs alone will constitute the hypoxia-responsive regulatory nucleic acid.

The hypoxia-responsive regulatory nucleic acid of the invention is hypoxia-responsive, meaning that expression of the nucleic acid molecule operably linked thereto is preferentially induced under hypoxic conditions. This advantageously allows expression to be induced only in hypoxic regions of the body, for example in solid tumours, hypoxic tissues and hypoxic organs (geographic targeting); or during certain periods of time, such as periods of hypoxia, ischemia (temporal targeting); or in response to certain environmental conditions, for example when conditions are hypoxic (environmental targeting or triggered targeting). Reference herein to “preferential” expression is taken to mean expression being driven under hypoxic conditions in preference to normoxic conditions. Although regulatory nucleic acids sometimes have “leaky” expression, the hypoxia-responsive regulatory nucleic acids of the invention showed no evidence of activation in normoxic conditions or tissues both in vitro and in vivo.

Furthermore, in a hypoxic environment, the regulatory nucleic acids of the invention are unexpectedly and advantageously stronger than some of the strongest lentiviral and retroviral promoters in current use, such as the SFG promoter. As a result, increased expression levels at the site of a tumour (i.e. in a hypoxic environment) is possible when using the regulatory nucleic acids of the invention compared to the expression levels seen when using conventional retroviral and lentiviral promoters in a hypoxic environment. This makes the use of the regulatory nucleic acids of the invention particularly advantageous when, for example, targeting in transient or low-level hypoxia, or when delivery of high loads of a therapeutic agent specifically in a hypoxic microenvironment is required, or when targeting low-density antigens, or when using a weak therapeutic agent, such as a weak CAR.

Use of the hypoxia-responsive regulatory nucleic acids of the invention need not be limited to applications where expression at the site of a tumour is desired. They may be used for any application where hypoxia-responsive expression is desired.

The term “regulatory nucleic acid” as defined herein refers to a nucleic acid capable of driving expression of a nucleic acid molecule operably linked thereto, “driving expression” referring to the initiation of transcription. Expression of the nucleic acid molecule which is operably linked to the regulatory nucleic acid is also dependent upon regulation of transcription, which regulation determines factors such as the strength of expression (as determined, for example, by the number of transgenes expressed per cell), where the nucleic acid molecule is expressed (e.g. tissue-specific expression), and when the nucleic acid molecule is expressed (e.g. inducible expression).

A “hypoxia-responsive regulatory nucleic acid” as defined herein is therefore capable of preferentially driving expression of a nucleic acid molecule operably linked thereto under conditions of hypoxia.

Regulation of expression may be mediated via transcriptional control elements, which are generally embedded in the nucleic acid sequence 5′-flanking or upstream of the expressed nucleic acid molecule. This upstream nucleic acid region is often referred to as a “promoter” since it promotes the binding, formation and/or activation of a transcription initiation complex and therefore is capable of driving and/or regulating expression of the 3′ downstream nucleic acid molecule.

The term “promoter” as used herein refers to regulatory nucleic acids capable of effecting (driving and/or regulating) expression of the sequences to which they are operably linked. A “promoter” encompasses transcriptional regulatory nucleic acids derived from a classical genomic gene. Usually a promoter comprises a TATA box, which is capable of directing the transcription initiation complex to the appropriate transcription initiation start site. However, some promoters do not have a TATA box (TATA-less promoters), but are still fully functional for driving and/or regulating expression. A promoter may additionally comprise a CCAAT box sequence and additional regulatory elements (i.e. upstream activating sequences or cis-elements such as enhancers and silencers). The terms (hypoxia-responsive) “regulatory nucleic acid”, “regulatory sequence” and “promoter” are used interchangeably herein. The regulatory nucleic acid may be “isolated”, i.e. removed from its original source.

Reference herein to being “operably linked” to a promoter or to a regulatory nucleic acid refers to the arrangement and relative positioning of the promoter/regulatory nucleic acid and the nucleic acid molecule to be expressed, such that the promoter/regulatory nucleic acid is able to drive expression of the nucleic acid molecule. The “nucleic acid molecule” may suitably be a gene, transgene, coding or non-coding sequence, RNA molecule (e.g. mRNA or RNA molecules for silencing, such as (shRNA, RNAi), micro-RNA regulation (miR), catalytic RNA, antisense RNA, RNA aptamers, etc.), an expression vector, TCR, CAR (first, second, third, fourth or any subsequent generation of CAR), or any other nucleic acid sequence of interest.

The hypoxia-responsive regulatory nucleic acid may be a known regulatory sequence modified to include a plurality of HREs or to add additional HRE(s). The plurality of HREs may be positioned anywhere within a known promoter (which promoter may comprise additional regulatory elements such as upstream activating sequences or cis-elements such as enhancers and silencers) and may confer hypoxia-responsiveness or may enhance existing levels of hypoxia-responsiveness. The plurality of HREs may be insertions within the known promoter sequence and/or may substitute all or a part or parts of the known promoter. Additionally or alternatively, the plurality of HREs may be insertions within a known enhancer and/or may substitute all or a part or parts of the known enhancer. The plurality of HREs may be spatially separate or may be sequential, or a combination of both.

The promoter to be modified to include a plurality of HREs may be selected from prokaryotic or eukaryotic promoters, such as: SFG, hACTB, hEF-1alpha, CAG, CMV, HSV-TK, hACTB, hACTB-R, LTRs, EF1a, SV40, PGK1, Ubc, human beta actin, TRE, UAS, Ac5, Polyhedrin, CaMKIIa, GAL1,10, TEF1, GDS, ADH1, CaMV35S, Ubi, H1, U6, T7, T7lac, Sp6, araBAD, trp, lac, Ptac, pL, an NFAT-interacting promoter (such as an IL-2 promoter), including functional fragments and minimal versions thereof. Other promoters which may be modified to include a plurality of HREs include the promoters listed in Table 1 below from Powel et al., (Discov Med. 2015, 19 (102), 49-57), also including functional fragments and minimal versions of the promoters listed in Table 1.

The hypoxia-responsive regulatory nucleic acid of the invention may be a “hybrid promoter”, such as a chimeric promoter, which may in addition to the plurality of HREs comprise a part or parts, preferably functional part(s), from another promoter. Examples of such parts include minimal promoters, additional regulatory elements to further enhance activity and/or to alter spatial and/or temporal expression pattern.

TABLE 1
Comparison of Selected Ubiquitous and Cell-specific Promoters.

| | | Relative | Size | |
|---|---|---|---|---|
| Promoter | Specificity | Strength | (bps) | Reference(s) |
| CMV | Ubiquitous | +++ | 750-800 | Xu et al., 2001; |
| | | | | Gray et al., 2011 |
| CBA | Ubiquitous | +++ | 248- | Klein et al., 2002; |
| (including | | | 1,600 | Ohlfest et al., |
| derivatives: | | | | 2005; Gray et al., |
| CAG, | | | | 2011 |
| CBh, etc.) | | | | |
| EF-1α | Ubiquitous | ++ | 2,500 | Gill et al., 2001; |
| | | | | Xu et al., 2001; |
| | | | | Ikeda et al., |
| | | | | 2002; Gilham |
| | | | | et al., 2010 |
| PGK | Ubiquitous | ++ | 426 | Gilham et al., 2010 |
| UBC | Ubiquitous | + | 403 | Gill et al., 2001; |
| | | | | Qin et al., 2010 |
| GUSB (hGBp) | Ubiquitous | + | 378 | Husain et al., 2009 |
| UCOE | Ubiquitous | ++ | 600- | Antoniou et al., 2013 |
| (Promoter of | | | 2,500 | |
| HNRPA2B1- | | | | |
| CBX3) | | | | |
| hAAT | Liver | ++ | 347- | Van Linthout et al., |
| | | | 1,500 | 2002; Cunningham |
| | | | | et al., 2008 |
| TBG | Liver | ++ | 400 | Yan et al., 2012 |
| Desmin | Skeletal | +++ | 1,700 | Talbot et al., 2010 |
| | muscle | | | |
| MCK | Skeletal | ++ | 595- | Wang et al., 2008; |
| | muscle | | 1,089 | Talbot et al., |
| | | | | 2010; Katwal et |
| | | | | al., 2013 |
| C5-12 | Skeletal, | ++ | 312 | Wang et al., 2008 |
| | cardiac, and | | | |
| | diaphragm | | | |
| NSE | Neuron | +++ | 300- | Xu et al., 2001 |
| | | | 2,200 | |
| Synapsin | Neuron | + | 470 | Kügler et al., 2003; |
| | | | | Hioki et al., 2007; |
| | | | | Kuroda et |
| | | | | al., 2008 |
| PDGF | Neuron | +++ | 1,400 | Patterna et al., 2000; |
| | | | | Hioki et al., 2007 |
| MeeP2 | Neuron | + | 229 | Rastegar et al., 2009; |
| | | | | Gray et al., 2011 |
| CaMKII | Neuron | ++ | 364- | Hioki et al., 2007; |
| | | | 2,300 | Kuroda et al., 2008 |
| mGluR2 | Neuron | + | 1,400 | Brené et al., 2000; |
| | | | | Kuroda et al., 2008 |
| NFL | Neuron | + | 650 | Xu et al., 2001 |
| NFH | Neuron | + | 920 | Xu et al., 2001 |
| nβ2 | Neuron | + | 650 | Xu et al., 2001 |
| PPE | Neuron | + | 2,700 | Xu et al., 2001 |
| Enk | Neuron | + | 412 | Xu et al., 2001 |
| EAAT2 | Neuron and | ++ | 966 | Su et al., 2003; |
| | astrocyte | | | Kuroda et al., 2008 |
| GFAP | Astrocyte | ++ | 681- | Brenner et al., 1994; |
| | | | 2,200 | Xu et al., 2001; |
| | | | | Lee et al., |
| | | | | 2008; Dirren et al., |
| | | | | 2014 |
| MBP | Oligo | ++ | 1,900 | Chen et al., 1998 |

Note:
Cell type specificity, relative strength (+ being the weakest and +++ being the strongest), size, and relevant references for commonly used promoters.

Each single HRE element (of the plurality of HREs) independently comprises, consists essentially of, or consists of, in any order, at least one HIF-binding site (HBS) and optionally at least one HIF ancillary site (HAS), optionally wherein said HBS and HAS are separated by a linker. Suitably the HRE may further comprise an HNF-4 site.

Although HREs comprising both HBS and HAS are preferred, the presence of the HAS is optional. Therefore, any reference herein to HREs also includes the option where the HRE has no HAS element.

HIF binding site (HBS):
(SEQ ID NO: 1)
5′-(A/G)CGT(G/C)-3′.
The HBS may optionally be ACGTG.
HIF ancillary site (HAS):
(SEQ ID NO: 2)
5′-CA(C/G)(G/A)(T/C/G)-3′.
The HAS may optionally be CACAG.
HNF-4 site:
(SEQ ID NO: 3)
5′-TGACCT-3′.

The HBS and HAS (if present) may be separated by a linker which may be rigid or flexible. Suitably, the linker is at least 6 nucleotides in length, optionally more than 8 nucleotides in length. Preferably, the linker is 6 or 8 nucleotides in length.

The linker may correspond to linkers naturally found in the promoter region of oxygen-responsive genes. An example of a suitable linker is given in SEQ ID NO: 4 (5′-GTCTCA-3′). Other suitable linkers are well known in the art and a person skilled in the art is familiar with the principles of linker design.

Table 2 below shows representative, but non-limiting, examples of HREs. The gene source from which the HRE is derived is shown in the left-hand column. The HBS and HAS (where present) is shown in bold.

TABLE 2
HREs from various gene sources

| Gene | Putative HRE with HBS | SEQ |
|---|---|---|
| source | and HAS highlighted | ID NO |
| hEPO | GGGCCCTACGTGC | SEQ ID |
| | TGTCTCACACAGC | NO: 5 |
| mEPO | GGGCCCTACGTGC | SEQ ID |
| | TGCCTCGCATGGC | NO: 6 |
| hPGK | TGTCACGTCCTGC | SEQ ID |
| | ACGACGCGAGTA | NO: 7 |
| mPGK | CGCGTCGTGCAGG | SEQ ID |
| | ACGTGACAAAT | NO: 8 |
| mLDH | CCAGCGGACGTGC | SEQ ID |
| | GGGAACCCACGTG | NO: 9 |
| | TAGG | |
| Glucose | TCCACAGGCGTGC | SEQ ID |
| trpt | CGTCTGACACGCA | NO: 10 |
| hVEGF | CCACAGTGCATAC | SEQ ID |
| | GTGGGCTCCAACA | NO: 11 |
| | GGTCCTCTT | |
| mVEFG | TACGTGGG | SEQ ID |
| | (conserved | NO: 12 |
| | human, | |
| | mouse, rat) | |
| rVEGF | ACAGTGCATACGT | SEQ ID |
| | GGGCTTCCACA | NO: 13 |
| hNOS | ACTACGTGCTGCC | SEQ ID |
| | TAGG | NO: 14 |
| hAldolase | CCCCTCGGACGTG | SEQ ID |
| | ACTCGGACCACAT | NO: 15 |
| hEnolase | ACGCTGAGTGCGT | SEQ ID |
| | GCGGGACTCGGAG | NO: 16 |
| | TACGTGACGGA | |
| mHeme | CGGACGCTGGCGT | SEQ ID |
| Oxygenase | GGCACGTCCTCTC | NO: 17 |

In addition to the HRE-containing genes shown in Table 2 above, other gene sources include: aldolase A, aldolase C, HIF-1β, HIF-2β, CTLA-4, PHD2, PHD3, enolase 1, enolase 2, glyceraldehyde-3-phosphate dehydrogenase, glucose phosphate isomerase 1, HIF-3α, 1L-10, interferon-γ, lymphocyte activation gene 3, mitochondrially encoded 12S rRNA, 6-phosphofructo-2-kinase/fructose-2,6-biphosphatase 3, phosphofructokinase; phosphoglycerate kinase 1, phosphoglucomutase 2, pyruvate kinase, perforin 1, glut1, glut3, triosephosphate isomerase 1, vascular endothelial growth factor A, Von Hippel-Lindau tumour suppressor. The aforementioned genes were shown by Gropper et al., 2017 (Cell Reports 20, 2547-2555) to be upregulated in T-cells upon exposure to hypoxia. The HREs included in the hypoxia-responsive regulatory nucleic acid may therefore be derived from any of the aforementioned genes or any of the genes listed in Table 2. Alternatively, the HREs included in the hypoxia-responsive regulatory nucleic acid may be artificially synthesised.

The hypoxia-responsive regulatory nucleic acid may comprise, essentially consist of, or consist of at least one or a plurality of sequences shown in Table 2 (SEQ ID NOs 5-17) or sequences having at least 70%, 75%, 80%, 85%, 90%, 91%, 92%, 93%, 94%, 95%, 96%, 97%, 98%, 99% or more sequence identity to any of SEQ ID NOs 7-19, and which sequences comprise, essentially consist of, or consist of at least the HBS (and optionally also the HAS) as shown in Table 1 or as defined herein.

The hypoxia-responsive regulatory nucleic acid comprises, essentially consists of, or consists of a plurality of HREs, with each individual HRE element comprising, essentially consisting of, or consisting of any combination of the following, in any order:
(i) at least one, two, three, four, five, six, seven, eight, nine, ten, eleven, twelve, thirteen, fourteen, fifteen or more HIF-binding sites (HBS), for example as represented by SEQ ID NO: 1, and optionally
(ii) at least one, two, three, four, five, six, seven, eight, nine, ten, eleven, twelve, thirteen, fourteen, fifteen or more HIF ancillary sites (HAS), for example as represented by SEQ ID NO: 2, and optionally
(iii) at least one, two, three, four, five, six, seven, eight, nine, ten, eleven, twelve, thirteen, fourteen, fifteen or more HNF-4 sites, for example as represented by SEQ ID NO: 3.

The hypoxia-responsive regulatory nucleic acid may comprise, essentially consist of, or consist of at least one, two, three, four, five, six, seven, eight, nine, ten, eleven, twelve, thirteen, fourteen, fifteen, sixteen, seventeen, eighteen, nineteen, twenty or more copies of SEQ ID NO: 1, optionally together with at least one, two, three, four, five, six, seven, eight, nine, ten, eleven, twelve, thirteen, fourteen, fifteen, sixteen, seventeen, eighteen, nineteen, twenty or more copies of SEQ ID NO: 2, and further optionally at least one, two, three, four, five, six, seven, eight, nine, ten, eleven, twelve, thirteen, fourteen, fifteen, sixteen, seventeen, eighteen, nineteen, twenty or more copies of SEQ ID NO: 3.

The “plurality” of HREs as defined herein is taken to mean at least two, three, four, five, six, seven, eight, nine, ten, eleven, twelve, thirteen, fourteen, fifteen, sixteen, seventeen, eighteen, nineteen, twenty or more copies of a single HRE element, a single HRE element being as defined herein.

Single (individual) HRE elements making up the plurality of HREs may be spatially separate (e.g. separated by elements such as enhancers, linkers, intervening sequences), or may be sequential, or a combination of both. Advantageously, the strength of the hypoxia-responsiveness may be tailored according to needs with an increase in the number of HREs correlating with an increase in hypoxia-responsiveness.

According to one embodiment, the hypoxia-responsive regulatory nucleic acid or plurality of HREs comprises, consists essentially of, or consists of three sequential “HBS-linker-HAS” sequences, i.e. HBS-linker-HAS-linker-HBS-linker-HAS-linker-HBS-linker-HAS. The linker being as defined herein or any suitable linker. In an alternative embodiment, there is no linker or wherein not every HBS-HAS is separated by a linker. In an alternative embodiment, there is no HAS element.

According to one embodiment, the hypoxia-responsive regulatory nucleic acid or plurality of HREs comprises, consists essentially of, or consists of six sequential “HBS-linker-HAS” sequences, i.e. HBS-linker-HAS-linker-HBS-linker-HAS-linker-HBS-linker-HAS-linker-HBS-linker-HAS-linker-HBS-linker-HAS-linker-HBS-linker-HAS. The linker being as defined herein or any suitable linker. In an alternative embodiment, there is no linker or wherein not every HBS-HAS is separated by a linker.

In an alternative embodiment, there is no HAS element.

According to one embodiment, the hypoxia-responsive regulatory nucleic acid or plurality of HREs comprises, consists essentially of or consists of nine sequential “HBS-linker-HAS” sequences, i.e. HBS-linker-HAS-linker-HBS-linker-HAS-linker-HBS-linker-HAS-linker-HBS-linker-HAS-linker-HBS-linker-HAS-linker-HBS-linker-HAS-linker-HBS-linker-HAS-linker-HBS-linker-HAS-linker-HBS-linker-HAS. The linker as being defined herein or any suitable linker. In an alternative embodiment, there is no linker or wherein not every HBS-HAS is separated by a linker. In an alternative embodiment, there is no HAS element.

The parts making up each individual HRE element, i.e. the HBS, and optionally the HAS and further optionally HNF-4, may be in any order. The parts may be positioned sequentially and/or spatially separate, such as through the use of suitable linkers, intervening sequences etc. Sequential positioning is also referred to herein as “in tandem” or “stacked”.

HREs or the parts making up an HRE (i.e. the HBS, and optionally HAS and further optionally HNF-4) may suitably be derived from any oxygen-responsive gene, preferably from a mammalian gene source, such as a human gene source, or they may be artificially synthesised. Examples of such oxygen-responsive genes include, among others, the genes listed in Table 2; genes listed hereinabove as shown by Gropper et al., 2017 (Cell Reports 20, 2547-2555) to be upregulated in T-cells upon exposure to hypoxia; erythropoietin (EPO), vascular endothelial growth factor (VEGF), phosphoglycerate kinase (PGK), glucose transporters (e.g. Glut-1), lactate dehydrogenase (LDH), aldolase (ALD), enolase (e.g. ENO3), glyceraldehyde-3-phosphate dehydrogenase (GAPDH), nitric oxide synthetase (NOS), heme oxygenase, muscle glycolytic enzyme pyruvate kinase (PKM), endothelin-1 (ET-1), including orthologues or paralogues of any of the aforementioned. “Orthologues” and “paralogues” are two forms of homology which encompass evolutionary concepts used to describe ancestral relationships of genes. The term “paralogue” relates to gene-duplications within the genome of a species leading to paralogous genes. The term “orthologue” relates to homologous genes in different organisms due to speciation. Orthologues and paralogues may readily be identified by a person skilled in the art using a (reciprocal) blast search.

The plurality of HREs may be placed anywhere within an expression vector, retroviral vector, or lentiviral vector e.g. pELNS etc., or any vector suitable for expressing a CAR. Optionally, the plurality of HREs are placed in a retroviral expression vector, for example, anywhere in the promoter or long terminal repeats (LTR) of a retroviral promoter. The plurality of HREs may be placed anywhere in the LTR for example, and/or may be juxtaposed to the open reading frame (ORF). The plurality of HREs may for example substitute substantially all or a part of the LTRs, enhancer and/or promoter with HREs. Optionally, the 3′ end of the LRT is modified to comprise a plurality of HREs. Optionally the 3′ LTR of the SFG retroviral vector is modified to replace substantially the entirety of the natural enhancer with a plurality of HREs, optionally whilst retaining the natural promoter or a part thereof.

SEQ ID NO: 18 below shows the unmodified 3′ LTR in the SFG retroviral vector.

SEQ ID NO: 18
CTGAATATGGGCCAAACAGGATATCTGTGGTAAGC
AGTTCCTGCCCCGGCTCAGGGCCAAGAACAGATGG
AACAGCTGAATATGGGCCAAACAGGATATCTGTGG
TAAGCAGTTCCTGCCCCGGCTCAGGGCCAAGAACA
GATGGTCCCCAGATGCGGTCCAGCCCTCAGCAGTT
TCTAGAGAACCATCAGATGTTTCCAGGGTGCCCCA
AGGACCTGAAATGACCCTGTGCCTTATTTGAACTA
ACCAATCAGTTCGCTTCTCGCTTCTGTTCGCGCGC
TTCTGCTCCCCGAGCTCAATAAAAGAGCCCACAAC
CCCTCACTCGGGGCGCCAGTCCTCCGATTGACTGA
GTCGCCCGGGTACCCGTGTATCCAATAAACCCTCT
TGCAGTTGCATCCGACTTGTGGTCTCGCTGTTCCT
TGGGAGGGTCTCCTCTGAGTGATTGACTACCCGTC
AGCGGGGGTCTTTCA

The MLV enhancer region of the SFG retroviral vector modified to include 9 HREs is shown below. SEQ ID NO: 19 below shows the sequence of the HRE modified 3′ LTR. The lower case section shows nine sequentially placed HREs, with a single HRE element being indicated in bold.

SEQ ID NO: 19
CTAGCggccctacgtgctgtctcacacagcctgtc
tgacggccctacgtgctgtctcacacagcctgtct
gacggccctacgtgctgtctcacacagcctgtctg
acggccctacgtgctgtctcacacagcctgtctga
cggccctacgtgctgtctcacacagcctgtctgac
ggccctacgtgctgtctcacacagcctgtctgacg
gccctacgtgctgtctcacacagcctgtctgacgg
ccctacgtgctgtctCACACAGCCTGTCTGACGGC
CCTACGTGCTGTCTCACACAGCCTGTCTGACtCTA
GAGAACCATCAGATGTTTCCAGGGTGCCCCAAGGA
CCTGAAATGACCCTGTGCCTTATTTGAACTAACCA
ATCAGTTCGCTTCTCGCTTCTGTTCGCGCGCTTCT
GCTCCCCGAGCTCAATAAAAGAGCCCACAACCCCT
CACTCGGGGCGCCAGTCCTCCGATTGACTGAGTCG
CCCGGGTACCCGTGTATCCAATAAACCCTCTTGCA
GTTGCATCCGACTTGTGGTCTCGCTGTTCCTTGGG
AGGGTCTCCTCTGAGTGATTGACTACCCGTCAGCG
GGGGTCTTTCA

SEQ ID NOs 20 to 25 annotate the component parts of SEQ ID NOs 18 and 19. As would be apparent to a person skilled in the art, not all the component parts are necessary for function. Also, one or more of the component parts represented by SEQ ID NOs 20 to 25 may be used to create hybrid promoters as defined herein.

MLV Promoter:
SEQ ID NO: 20
GAACCATCAGATGTTTCCAGGGTGCCCCAAGGACC
TGAAATGACCCTGTGCCTTATTTGAACTAACCAAT
CAGTTCGCTTCTCGCTTCTGTTCGCGCGCTTCTGC
TCCCCGAGCTCAATAAAAGAGCCCACAACCCCTCA
CTCGG
CCAAT box:
SEQ ID NO: 21
CTAGAGAACCATCAGATGTTTCCAGGGTGCCCCAA
GGACCTGAAATGACCCTGTGCCTTATTTGAACTAA
CCAATCAGTTCGCTTCTCGCTTCTGTTCGCGCGCT
TC
TATA box:
SEQ ID NO: 22
TGCTCCCCGAGCTCAATAAAAGAGCCCACAACCCC
TCACTCGGGGCGCCAGTCCTCCGAT
Poly A site:
SEQ ID NO: 23
TGACTGAGTCGCCCGGGTACCCGTGTATCCAATAA
ACCCTCTTGCAGTTGCA
RNA template for strong-stop-cDNA:
SEQ ID NO: 24
GCGCCAGTCCTCCGATTGACTGAGTCGCCCGGGTA
CCCGTGTATCCAATAAACCCTCTTGCAGTTGCATC
CGACTTGTGGTCTCGCTGTTCCTTGGGAGGGTCTC
CTCTGAGTGATTGACTACCCGTCAGCGGGGGTCTT
TCA

11-Base Inverted Repeat: SEQ ID NO: 25

GGGGTCTTTCA

Alternatively, the plurality of HREs may themselves have sufficient regulatory function/promoter activity, i.e. the capability to initiate transcription and to drive and regulate expression of the nucleic acid molecule operably linked thereto, in which case the plurality of HREs alone will constitute the hypoxia-responsive regulatory nucleic acid. Each individual HRE element of the plurality of HREs may be spatially separate (e.g. separated by elements such as enhancers, linkers, intervening sequences), or may be sequential, or a combination of both.

SEQ ID NO: 26 below shows an example where the plurality of HREs themselves constitute the hypoxia-responsive regulatory nucleic acid. Nine sequential copies of HREs are shown with a single HRE element being in bold.

SEQ ID NO: 26
GGCCCTACGTGCTGTCTCACACAGCCTGTCT
GACGGCCCTACGTGCTGTCTCACACAGCCTG
TCTGACGGCCCTACGTGCTGTCTCACACAGC
CTGTCTGACGGCCCTACGTGCTGTCTCACAC
AGCCTGTCTGACGGCCCTACGTGCTGTCTCA
CACAGCCTGTCTGACGGCCCTACGTGCTGTC
TCACACAGCCTGTCTGACGGCCCTACGTGCT
GTCTCACACAGCCTGTCTGACGGCCCTACGT
GCTGTCT

SEQ ID NO: 27 below shows an example of a single HRE element, with the HBS and HAS shown in bold. The hypoxia-responsive regulatory nucleic acid may comprise, essentially consist of, or consist of multiple copies of SEQ ID NO: 27 or a part thereof comprising at least the HBS and optionally the HAS element, for example, at least 2, 3, 4, 5, 6, 7, 8, 9, 10, 11, 12, 13, 14, 15, 16, 17, 18, 19, 20 or more copies of SEQ ID NO: 27 or a part thereof. Each individual HRE copy may be spatially separate (e.g. separated by elements such as enhancers, linkers, intervening sequences), or may be sequential (also referred to herein as “in tandem” or “stacked”), or a combination of both.

SEQ ID NO: 27
GGCCCTACGTGCTGTCTCACACAGCCTGTCTGAC

The present invention also provides functional fragments of the regulatory nucleic acids of the invention, which “functional fragments”, as defined herein, comprise, consist essentially of, or consist of a plurality of HREs and which retain the capability to drive and to regulate expression of the nucleic acid molecule operably linked thereto. The functional fragments retain the capability to drive and/or to regulate expression in the same way (although possibly not to the same extent) as the unmodified sequence from which they are derived, or on which the fragment is based. Suitable functional fragments may be tested for their capability to drive and/or regulate expression using standard techniques well known to the skilled person. Functional fragments comprise at least 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 or more contiguous nucleotides of the sequence from which they are derived. In a particular embodiment, the functional fragment is a functional fragment of SEQ ID NO: 18, 19, 20, 21, 22, 23, 24, 25, 26 or 27 and which functional fragment comprises or consists of a plurality of HREs as defined herein.

According to another embodiment, the hypoxia-responsive regulatory nucleic acids are represented by or comprise, essentially consist of, or consist of SEQ ID NO: 18, 19, 20, 21, 22, 23, 24, 25, 26 or 27 or a functional fragment thereof or the complement thereof.

The hypoxia-responsive regulatory nucleic acid may also comprise, essentially consist of, or consist of sequences capable of hybridizing under stringent hybridization conditions with any of SEQ ID NO: 18, 19, 20, 21, 22, 23, 24, 25, 26 or 27 or with functional fragments as defined herein, which hybridizing sequences comprise, consist essentially of, or consist of a plurality of HREs and retain the capability to drive and to regulate expression of the nucleic acid molecule operably linked thereto. Hybridization under stringent conditions refers to the ability of a nucleic acid molecule to hybridize to a target nucleic acid molecule under defined conditions of temperature and salt concentration. Typically, stringent hybridization conditions are no more than 25° C. to 30° C. (for example, 20° C., 15° C., 10° C. or 5° C.) below the melting temperature (Tₘ) of the native duplex. Methods of calculating Tₘ are well known in the art. By way of non-limiting example, representative salt and temperature conditions for achieving stringent hybridization are: 1×SSC, 0.5% SDS at 65° C. The abbreviation SSC refers to a buffer used in nucleic acid hybridization solutions. One liter of the 20× (twenty times concentrate) stock SSC buffer solution (pH 7.0) contains 175.3 g sodium chloride and 88.2 g sodium citrate. A representative time period for achieving hybridization is 12 hours.

The hypoxia-responsive regulatory nucleic acid may comprise or consist of a homologue having at least 70%, 75%, 80%, 85%, 90%, 91%, 92%, 93%, 94%, 95%, 96%, 97%, 98%, 99% or more sequence identity to SEQ ID NO: 18, 19, 20, 21, 22, 23, 24, 25, 26 or 27 or to functional fragments thereof, which homologues comprise, essentially consist of, or consist of a plurality of HREs. The percentage identity may be calculated using an alignment program. Preferably a pair wise global alignment program may be used, which implements the algorithm of Needleman-Wunsch (J. Mol. Biol. 48: 443-453, 1970). This algorithm maximizes the number of matches and minimizes the number of gaps. Such programs are for example GAP, Needle (EMBOSS package), stretcher (EMBOSS package) or Align X (Vector NTI suite 5.5) and may use the standard parameters (for example gap opening penalty 15 and gap extension penalty 6.66). Alternatively, a local alignment program implementing the algorithm of Smith-Waterman (Advances in Applied Mathematics 2, 482-489 (1981)) may be used. Such programs are for example Water (EMBOSS package) or matcher (EMBOSS package).

Other variants of the hypoxia-responsive regulatory nucleic acid of the invention or variants of SEQ ID NO: 18, 19, 20, 21, 22, 23, 24, 25, 26 or 27 include mutational variants, substitutional variants, insertional variants, derivatives, variants including intervening sequences, splice variants and allelic variants, which variants comprise or consist of a plurality of HREs.

A “mutation variant” of a nucleic acid may readily be made using recombinant DNA manipulation techniques or nucleotide synthesis. Examples of such techniques include site directed mutagenesis via M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols. Alternatively, the nucleic acid of the present invention may be randomly mutated.

A “substitutional variant” refers to those variants in which at least one residue in the nucleic acid sequence has been removed and a different residue inserted in its place. Nucleic acid substitutions are typically of single residues, but may be clustered depending upon functional constraints placed upon the nucleic acid sequence; insertions usually are of the order of about 1 to about 10 nucleic acid residues, and deletions can range from about 1 to about 20 residues.

An “insertional variant” of a nucleic acid is a variant in which one or more nucleic acid residues are introduced into a predetermined site in that nucleic acid. Insertions may comprise 5′-terminal and/or 3′-terminal fusions as well as intra-sequence insertions of single or multiple nucleotides. Generally, insertions within the nucleic acid sequence will be smaller than 5′- or 3′-terminal fusions, of the order of about 1 to 10 residues. Examples of 5′- or 3′-terminal fusions include the coding sequences of binding domains or activation domains of a transcriptional activator as used in the yeast two-hybrid system or yeast one-hybrid system, or of phage coat proteins, (histidine)6-tag, glutathione S-transferase-tag, protein A, maltose-binding protein, dihydrofolate reductase, Tag●100 epitope, c-myc epitope, FLAG®-epitope, lacZ, CMP (calmodulin-binding peptide), HA epitope, protein C epitope and VSV epitope.

The term “derivative” of a nucleic acid may comprise substitutions, and/or deletions and/or additions of naturally and non-naturally occurring nucleic acid residues compared to the natural nucleic acid. Derivatives may, for example, comprise methylated nucleotides, or artificial nucleotides.

The regulatory sequence may be interrupted by an intervening sequence. With “intervening sequence” is meant any nucleic acid or nucleotide, which disrupts another sequence. Examples of intervening sequences comprise introns, nucleic acid tags, T-DNA and mobilizable nucleic acids sequences such as transposons or nucleic acids that can be mobilized via recombination. Examples of particular transposons comprise Ac (activator), Ds (Dissociation), Spm (suppressor-Mutator) or En. In case the intervening sequence is an intron, alternative splice variants may arise. The term “alternative splice variant” as used herein encompasses variants of a nucleic acid sequence in which intervening introns have been excised, replaced or added. Such splice variants may be found in nature or may be manmade.

The hypoxia-responsive regulatory nucleic acid of the invention is capable of driving expression under conditions of hypoxia, which as defined herein, is taken to mean O₂ concentration of below 5% (such as less than 4%, 3%, 2%, 1%, 0.5% 0.25% or 0.1% or the mmHg equivalent) or reduced O₂ availability relative to O₂ availability or partial pressure of a corresponding non-cancerous organ, tissue or cells. Conversely, “normoxia” as defined herein is taken to mean O₂ concentrations above 5% or O₂ availability associated with healthy organs. A person skilled in the art would readily be able to determine whether any given environment is hypoxic or normoxic. Depending on the envisaged use of the promoter, the skilled person would be able to use a differing number of HRE copies in order to adjust the degree of hypoxia responsiveness, with an increase in HRE copies correlating to an increase in hypoxia responsiveness.

The hypoxia-responsive regulatory nucleic acid is capable of driving expression of a nucleic acid molecule which may suitably be a gene, transgene, coding or non-coding sequence, RNA molecule (e.g. mRNA or RNA molecules for silencing, such as (shRNA, RNAi), micro-RNA regulation (miR), catalytic RNA, antisense RNA, RNA aptamers, etc.), an expression vector, and engineered receptor such as a CAR (first, second, third, fourth or any subsequent generation of CAR) or TCR, or any other sequence of interest.

Engineered Receptor

In one aspect, the hypoxia-responsive regulatory nucleic acid drives expression of an engineered receptor that, when expressed in an immunoresponsive cell, confers on the cell a predetermined antigen specificity and, upon binding of the cell to the predetermined antigen, delivers to the cell an activation signal and, optionally, one or more costimulatory signals. In typical embodiments, the immunoresponsive cell is a Natural Killer cell, invariant NKT-cell, NK T-cell, B-cell, T-cell, such as cytotoxic T-cells, helper T-cells or regulatory T-cells, αβ T-cell, γδ T-cell, or myeloid-derived cells such as a macrophages or neutrophils, stem cells, induced pluripotent stem cells (iPSCs).

Operably linking the hypoxia-responsive regulatory nucleic acid to a polynucleotide that encodes the engineered receptor confers hypoxia-responsive expression to the engineered receptor and/or renders it suitable for targeting the immunoresponsive cell to a solid tumour mass.

The earliest chimeric antibody-TCR was made by Kuwana et al. 1987 (Biochemical and Biophysical Research Communications, Vol. 149, No. 3). One of the earliest CARs was developed by Zelig Eshhar et al. at the Weizmann Institute in Israel (Gross et al., 1989 (PNAS, Vol. 86, pp. 10024-10028); Eshhar et al., 1993 (PNAS, Vol. 90, pp. 720-724)). Based on their findings, the fusion of a Fab antigen binding region from an antibody with the intracellular TCR signalling domains gives rise to a chimeric receptor, which is functional when expressed on T-cells and delivers a TCR signal in response to a specified MHC/HLA independent antigen. The modular architecture of the CAR, which includes various functional domains, permits the choice of antigen specificity and to finely control signalling strength. CAR can comprise a single chain variable fragment (scFv), which contains the variable heavy (VH) and light chain (VL) regions of an antibody specific to a TAA or peptide ligand to a receptor or a fusion of peptides, a suitable spacer domain, for example, CD8, CD28 or IgG-Fc, others being well known in the art; a transmembrane domain and an endodomain. The spacer orients the scFv at an optimal distance from the T-cell plasma membrane for efficient signalling to occur. Apart from this, the spacer plays an important role in receptor homodimerization, flexibility and segregation and aggregation. The signalling endodomain is made of proteins that contain signal transduction motifs, which provide the co-stimulation for the native TCR activation. The endodomain can contain CD3ζ, FcRγ, CD28, OX40 and/or 4-1BB, amongst others, and the combination of these domains determines the generation of the chimeric receptor, which has become more sophisticated over time.

The hypoxia-responsive regulatory nucleic acid regulatory element according to the first aspect of the present invention may be used to drive and to regulate expression of any engineered receptor.

In addition, in certain embodiments, the engineered receptor, such as a CAR, comprises one or more Oxygen dependent Degradation Domains (ODDs), as defined herein, and at least one polypeptide with anti-tumour properties.

Due to the modular nature of CARs, the one or more ODDs or chimeric polypeptide may readily be included in any known CAR design: for example, they may be included in a first, second, third, fourth or subsequent generation of CAR; split CAR systems; TRUCKs or armoured CARs etc. Known CARs may be adapted to confer hypoxia responsiveness or to confer improved hypoxia responsiveness through the inclusion of one or more ODDs, as defined herein, and/or through the use of the hypoxia-responsive regulatory nucleic acids according to the first aspect of the invention.

First generation CARs are composed of an extracellular binding domain, a hinge region, a transmembrane domain, and one or more intracellular signalling domains. Commonly, the extracellular binding domain comprises a single-chain variable fragment (scFv) derived from a tumour antigen-reactive antibody and usually has high specificity to tumour antigen. A first generation CAR typically comprises the CD3ζ chain domain or a modified derivative thereof as the intracellular signalling domain, which is the primary transmitter of signals.

Second generation CARs also contain a co-stimulatory domain, such as CD28 and/or 4-1BB. The inclusion of an intracellular co-stimulatory domain improves T-cell proliferation, cytokine secretion, resistance to apoptosis, and in vivo persistence. The co-stimulatory domain of a second generation CAR is typically in cis with and upstream of the one or more intracellular signalling domains.

Third-generation CARs combine multiple co-stimulatory domains in cis with one or more intracellular signalling domains, to augment T-cell activity. For example, a third-generation CAR may comprise co-stimulatory domains derived from CD28 and 41BB, together with an intracellular signalling domain derived from CD3z. Other third-generation CARs may comprise co-stimulatory domains derived from CD28 and OX40, together with an intracellular signalling domain derived from CD3z.

Fourth-generation CARs (also known as TRUCKs or armoured CARs), combine the expression of a second-generation CAR with factors that enhance anti-tumoural activity (e.g., cytokines, co-stimulatory ligands, chemokines receptors or further chimeric receptors of immune regulatory or cytokine receptors). The factors may be in trans or in cis with the CAR, typically in trans with the CAR.

The CAR or nucleic acid encoding the CAR may additionally include other mechanisms to deal with off target effects, dose control, location and timing of activation. For example, the nucleic acid encoding the CAR may include suicide gene(s), such as herpes simplex virus thymidine kinase (HSV-TK) or inducible caspase 9 (iCas9), or other means to control off target effects. Other means for control of CAR activity include the use of a small molecule agent (e.g. as reported in Giordano-Attinese et al., 2020, Nature Biotechnology Letters). These control systems may be activated by an extracellular molecule to induce apoptosis of the immunoresponsive cell.

Another example includes a CAR designed to express two or more antigen-specific targeting regions (as defined herein). The CAR may be a split CAR system in which the therapeutic function of the CAR requires the presence of both a tumour antigen and a benign exogenous molecule. Such a system may be used in the present invention to control the deployment of the ODD.

In various embodiments, the engineered receptor is a first generation CAR, such as those described in Eshhar et al., Proc. Natl. Acad. Sci. USA (1993) 90(2):720-724.

In various embodiments, the engineered receptor is a co-stimulatory chimeric receptor, such as those described in Krause et al., J. Exp. Med. (1998) 188(4):619-26.

In various embodiments, the engineered receptor is a second generation CAR, such as those described in Finney et al., J. Immunol. (1998) 161(6):2791-7; Maher et al., Nat. Biotechnol. (2002) 20(1):70-75; Finney et al., J. Immunol. (2004) 172(1):104-113; and Imai et al., Leukemia (2005) 18(4):676-84.

In various embodiments, the engineered receptor is a third generation CAR, such as those described in Pule et al. (2005), Mol. Ther. 12(5):933-941; Geiger et al., Blood (2001) 98:2364-71; and Wilkie et al. J. Immunol. (2008) 180(7):4901-9.

In various embodiments, the engineered receptor is a tandem (Tan)CAR, as described in Ahmed et al., Mol. Ther. Nucleic Acids (2013) 2:e105.

In various embodiments, the engineered receptor is a TRUCK CAR, as described in Chmielewski et al., Cancer Res. (2011), 71:5697-5706 (2011).

In various embodiments, the engineered receptor is an Armoured CAR, as described in Pegram et al., Blood (2012) 119:4133-4141 and Curran et al., Mol. Ther. (2015) 23(4):769-78.

In various embodiments, the engineered receptor is a Switch Receptor, as described in WO 2013/019615.

In various embodiments, the engineered receptor is expressed in the cell with other engineered constructs.

In some of these embodiments, the engineered receptor is expressed in the cell with other engineered constructs to provide co-stimulation in cis and in trans, as described in Stephan et al. Nat. Med. (2007) 13(12):1440-49.

In some of these embodiments, the engineered receptor is expressed in the cell with other engineered constructs to provide dual-targeted CARs, such as those described in Wilkie et al., J. Clin. Immunol. (2012) 32(5):1059-70.

In some of these embodiments, the engineered receptor is expressed in the cell with other engineered constructs to provide inhibitory CARs (NOT gate), as described in Fedorov et al., Sci. Transl. Med. (2013) 5(215):215ra172.

In some of these embodiments, the engineered receptor is expressed in the cell with other engineered constructs to provide combinatorial CARs (AND gates), as described in Kloss et al., Nat. Biotechnol. (2013) 31(1):71-5 and WO 2014/055668.

In some of these embodiments, the engineered receptor is expressed in the cell with other engineered constructs to provide a Go-CAR T, as described in Foster et al., (2014), Abstract, bloodjournal.org/content/124/21/1121?sso-checked=true.

In some of these embodiments, the engineered receptor is expressed in the cell with other engineered constructs to provide engineered co-stimulation, as described in Zhao et al., Cancer Cell (2015) 28:415028.

In some of these embodiments, the engineered receptor is expressed in the cell with other engineered constructs to provide SynNotch/sequential AND gate as described in Roybal et al., Cell (2016) 164:770-79.

In certain preferred embodiments, the engineered receptor is expressed in the cell with other engineered constructs to provide a parallel CAR (pCAR), as described in WO 2017/021701. A pCAR may comprise a second generation chimeric antigen receptor comprising:
(a) a signalling region;
(b) a co-stimulatory signalling region;
(c) a transmembrane domain; and
(d) a binding element that specifically interacts with a first epitope on a target antigen; and a chimeric costimulatory receptor comprising
(e) a co-stimulatory signalling region which is different to that of (b);
(f) a transmembrane domain; and
(g) a binding element that specifically interacts with a second epitope on a target antigen.

In various embodiments, the engineered receptor is an engineered T-cell receptor, such as those described in WO 2010/026377; WO 2010/133828; WO 2011/001152; WO 20123/013913; WO 2013/041865; WO 2017/109496; WO 2017/163064; and WO 2018/234319.

In embodiments, the CAR comprises means to home to or infiltrate the tumour bed. For example, the CAR may comprise one or more chemokine receptors.

Further engineered receptors may be included. Additional engineered receptors may be designed to include means to home to or infiltrate the tumour bed. For example, an additional engineered receptor may comprise a chimeric cytokine receptor or a chemokine receptor.

As discussed further below, any known CAR design or type, such as any of the aforementioned, may be adapted to include the capacity for expression and regulation under hypoxic conditions through the use of one or more ODDs and/or through the use of the hypoxia-inducible regulatory sequence according to the first aspect of the invention.

In addition to use of the hypoxia-inducible regulatory sequence and optional inclusion of the one or more ODDs as discussed further below, the CAR will typically include the following known components described under I to IV below.

I. Extracellular Antigen-Specific Targeting Region (or Polypeptide with Anti-Tumour Properties)

In addition to the at least one ODD, the chimeric polypeptide comprises at least one polypeptide with anti-tumour properties, also referred to herein as an extracellular antigen-specific targeting region. The extracellular antigen-specific targeting region and the one or more ODDs may be linked.

Such proteins for delivery to a tumour include but are not limited to any one or more of the following: immune stimulating antibodies; surface or intracellular receptors that confer cell activation and tumour-killing capability; a T-cell Receptor (TCR).

The antigen-specific targeting region provides the CAR with the ability to bind a predetermined antigen of interest. The antigen-specific targeting region preferably targets an antigen of clinical interest. The antigen-specific targeting region may be any protein or peptide that possesses the ability to specifically recognise and bind to a biological molecule (e.g., a cell surface receptor or a component thereof). The antigen-specific targeting region includes any naturally occurring, synthetic, semi-synthetic, or recombinantly produced binding partner for a biological molecule of interest. Illustrative antigen-specific targeting regions include antibodies or antibody fragments or derivatives, extracellular domains of receptors, ligands for cell surface molecules/receptors, or receptor binding domains thereof, and tumour binding proteins.

In a preferred embodiment, the antigen-specific targeting region is, or is derived from, an antibody. An antibody-derived targeting domain can comprise a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a single-domain antibody (VHH). The antigen-specific targeting region may additionally or alternatively comprise or consist of or be derived from monobodies. In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another directly or via a peptide linker sequence.

Antigen binding regions of a CAR that specifically bind a predetermined antigen can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

Antigens which may be targeted by the present CAR include but are not limited to antigens expressed on cells associated with a solid cancer.

The antigen to targeted is not limited to but may be selected from one or more and any combination of the following and derivatives and variants thereof: extended ErbB family, Erbb1, Erbb3, Erbb4, Erbb2/HER-2, mucins, PSMA, CEA, mesothelin, GD2, MUC1, folate receptor, GPC3, CAIX, FAP, NY-ESO-1, gp100, PSCA, ROR1, PD-L1, PD-L2, EpCAM, EGFRvIII, CD19, GD3, CLL-1, ductal epithelial mucin, Gp36, TAG-72, glycosphingolipids, glioma-associated antigen, beta-hCG, AFP (alpha-fetoprotein) and lectin-reactive AFP, thyroglobulin, receptor for advanced glycation end products (RAGE), TERT, telomerase, carboxylesterase, M-CSF, PSA, survivin, PCTA-1, MAGE, CD22, IGF-1, IGF-2, IGF-1 receptor, MHC-associated tumour peptide, 5T4, tumour stroma-associated antigens, WT1, MLANA, CA 19-9, BCMA, □v□6 integrin, virus-specific antigens.

A preferred extracellular antigen-specific targeting region is T1E (Davies et al., 2012, Mol Med 18:565-576), SEQ ID NO: 32. Functional fragments and variant thereof, wherein the variant has at least 70%, 75%, 80%, 85%, 90%, 95% or more sequence identity to SEQ ID NO: 32, are also included.

T1E Peptide (Derived from Human TGFα and EGF); SEQ ID NO: 32

(SEQ ID NO: 32)
VVSHFNDCPLSHDGYCLHDGVCMYIEALDK
YACNOVVGYIGERCQYRDLKVWVELR.

II. Intracellular Signalling Domain (Also Referred to as an Endodomain)

Suitable intracellular signalling domains are known in the art and include, for example, any region comprising an Immune-receptor-Tyrosine-based-Activation-Motif (ITAM), as reviewed for example by Love et al. Cold Spring Harbor Perspect. Biol 2010 2(6)I a002485. In a particular embodiment, the signalling region comprises the intracellular domain of human CD3 [zeta] chain as described for example in U.S. Pat. No. 7,446,190, or a variant thereof.

The intracellular signalling domain may also be a transcription factor for indirect signalling.

The intracellular domain may be represented by SEQ ID NO: 33 or a functional fragment or variant thereof, wherein the variant has at least 70%, 75%, 80%, 85%, 90%, 95% or more sequence identity to SEQ ID NO: 33.

CD3z or CD3 zeta (intracellular domain); SEQ ID NO: 33

(SEQ ID NO: 33)
RVKFSRSADAPAYQQGQNQLYNELNLGRREEY
DVLDKRRGRDPEMGGKPRRKNPQEGLYNELQ
KDKMAEAYSEIGMKGERRRGKGHDGLYQGLS
TATKDTYDALHMQALPPR.

III. Transmembrane Domain

CARs are expressed on the surface of the cell membrane and therefore typically comprise transmembrane domains. Suitable transmembrane domains are known in the art and include for example, the transmembrane sequence from any protein which has a transmembrane domain, including any of the type I, type II or type III transmembrane proteins. The transmembrane domain of the CAR may also comprise an artificial hydrophobic sequence. The transmembrane domains of the CAR may be selected so as not to dimerize. Suitable transmembrane domains include CD8α, CD28, CD4 or CD3ζ transmembrane domains.

In an embodiment, the transmembrane domain is represented by SEQ ID NO: 34 or a functional fragment or variant thereof, wherein the variant has at least 70%, 75%, 80%, 85%, 90%, 95% or more sequence identity to SEQ ID NO: 34.

CD28 (Transmembrane Domain); SEQ ID NO: 34

(SEQ ID NO: 34)
IEVMYPPPYLDNEKSNGTIIHVKGKHLCPSPL
FPGPSKPFVVVLVVVGGVLACYSLLVTVAFII
FVVVRSKRSRLLHSDYMNMTPRRPGPTRKHYQ
PYAPPRDFAAYRS.

IV. Co-Stimulatory Domains

Suitable co-stimulatory domains are also well known in the art, and include members of the B7/CD28 family such as B7-1. B7-2, B7-H1, B7-H2, B7-H3, B7-H4, BT-H6, 87-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumour necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha or TNF RII; or members of the SLAM family such as 2B4, BLAME, CD2, CD2F-10, CD48, CD58, CD84, 00229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP12, Dectin-1, DPPIV, EphB6, Integrin alpha 4 beta 1, Integrin alpha 4 beta 7/LPAM-1, LAG-3 or TSLP R.

In embodiments, the CAR comprises a plurality of co-stimulatory domains, for example two or more co-stimulatory domains. In some embodiments the co-stimulatory domain is derived from CD28, 4-1BB and/or OX40.

In embodiments, the co-stimulatory domain is CD28 or is derived from CD28.

In embodiments, the co-stimulatory domain is 4-1BB or is derived from 4-1BB.

Chimeric Polypeptides Comprising ODD

According to one embodiment, the hypoxia-responsive regulatory nucleic acid is operably linked to a nucleic acid molecule encoding a chimeric polypeptide that comprises (i) one or more Oxygen-dependent Degradation Domains (ODD) and (ii) at least one polypeptide with anti-tumour properties.

The ODD may be derived from any ODD-containing protein, such as ATF-4, HIF1-alpha, HIF2-alpha and HIF3-alpha, which may be from a mammalian, such as human, source or may be artificially created.

The ODD may be represented by SEQ ID NO: 28 (X¹X²LEMLAPYIXMDDDX³X⁴X⁵), where “X¹⁻⁵” can be any amino acid residue. Optionally, X¹ is “L” or any conservative substitution; X² is “D” or any conservative substitution, X³ is “F” or any conservative substitution, X⁴ is “Q” or any conservative substitution, X⁵ is “L” or any conservative substitution.

Optionally, the ODD may be represented by SEQ ID NO: 29, 30 or 31, or homologues or variants thereof having at least 70%, 75%, 80%, 85%, 90%, 95% or more sequence identity to SEQ ID NO: 29, 30 or 31, wherein the homologue or variant comprises SEQ ID NO: 28.

SEQ ID NO: 29 (HIF1-Alpha Amino Acids 401-603, with SEQ ID NO: 37 in Bold)

APAAGDTIISLDFGSNDTETDDQQLEEVPLYNDVM
LPSPNEKLQNINLAMSPLPTAETPKPLRSSADPAL
NQEVALKLEPNPESLELSFTMPQIQDQTPSPSDGS
TRQSSPEPNSPSEYCFYVDSDMVNEFKLELVEKLF
AEDTEAKNPFSTQDTDLDLEMLAPYIPMDDDFQLR
SFDQLSPLESSSASPESASPQSTVTVFQ

SEQ ID NO: 30 (HIF1-Alpha Amino Acids 530-603, with SEQ ID NO: 37 in Bold)

EFKLELVEKLFAEDTEAKNPFSTQDTDLDLEMLAP
YIPMDDDFQLRSFDQLSPLESSSASPESASPQSTV
TVFQ

SEQ ID NO: 31 (HIF1-Alpha Amino Acids 530-653, with SEQ ID NO: 37 in Bold)

EFKLELVEKLFAEDTEAKNPFSTQDTDLDLEMLAP
YIPMDDDFQLRSFDQLSPLESSSASPESASPQSTV
TVFQQTQIQEPTANATTTTATTDELKTVTKDRMED
IKILIASPSPTHIHKETTS

Additionally or alternatively, the ODD may be encoded by a nucleic acid encoding SEQ ID NO: 29, 30 or 31, or homologues or variants thereof having at least 70%, 75%, 80%, 85%, 90%, 91%, 92%, 93%, 94%, 95%, 96%, 97%, 98%, 99% or more sequence identity to SEQ ID NO: 29, 30 or 31 and comprising SEQ ID NO: 28.

The “homologue” as defined herein has at least 70%, 75%, 80%, 85%, 90%, 91%, 92%, 93%, 94%, 95%, 96%, 97%, 98%, 99% or more sequence identity to SEQ ID NO: 29, 30 or 31, and comprises SEQ ID NO: 27. Identity in this context (and as referred to elsewhere in the present application) may be determined using the BLASTP computer program with SEQ ID NO 29, 30 or 31, for example, as the base sequence. The BLAST software is publicly available at http://blast.ncbi.nlm.nih.gov/Blast.cgi (accessible on 12 Mar. 2009).

More generally, unless stated otherwise, the term “variant” as referred to herein refers to a polypeptide sequence which is a naturally occurring polymorphic form of the basic sequence as well as synthetic variants, in which one or more amino acids within the chain are inserted, removed or replaced. The variant produces a biological effect which is similar to that of the basic sequence.

Amino acid substitutions may be regarded as “conservative” where an amino acid is replaced with a different amino acid in the same class with broadly similar properties. Non-conservative substitutions are where amino acids are replaced with amino acids of a different type or class.

Amino Acid Classes are Defined as Follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions may also be possible provided that these do not interrupt the function of the polypeptide as described above. Broadly speaking, fewer non-conservative substitutions will be possible without altering the biological activity of the polypeptides.

The hypoxia-responsive regulatory nucleic acid is operably linked to a nucleic acid molecule encoding a chimeric polypeptide comprising one or more ODDs. The chimeric polypeptide may comprise at least one, two, three, four, five or more ODDs, for example, as represented by any of SEQ ID NOs 29, 30 and 31, and homologues and variants thereof as defined herein and which comprise SEQ ID NO: 28. Where the use of more than one ODD is envisaged, they may be provided in the same construct or in separate constructs; if on the same construct, they may be sequential or spatially separate.

The one or more ODDs may be positioned anywhere in a polypeptide or nucleic acid (including RNA). For example, they may be positioned at the C- or N-terminal or anywhere in between the polypeptide chain, either directly attached to the polypeptide chain or linked to the polypeptide chain using linkers, the polypeptide having anti-tumour properties. Suitable linkers are well known in the art and may be rigid or flexible. In one embodiment, the ODD(s) may be comprised in a CAR, optionally fused to the C-terminal end of a CAR.

The polypeptides and nucleic acids encoding the same, the CARs and immunoresponsive cells of the invention are capable of dual sensing/dual expression, i.e. to cause activity or expression of the tumour-targeting polypeptide under conditions of hypoxia, such as found in the solid cancer environment, but with little or no activity or expression in a normoxic environment. This is thanks to the degradation of the polypeptide with anti-tumour properties as effected by the ODD(s) in combination with the expression driven by the hypoxia-responsive regulatory nucleic acid described in the first aspect of the invention.

The hypoxia-responsive regulatory nucleic acid according to the first aspect of the invention is capable of regulating expression of a nucleic acid molecule encoding a chimeric polypeptide comprising one or more Oxygen-Dependent Degradation Domains (ODD) and at least one polypeptide with anti-tumour properties. The expression of the chimeric polypeptide is controlled in a hypoxia-responsive manner thanks to the action of the regulatory sequence in combination with the one or more ODDs, wherein the stringency of the system can be adjusted, for example, by adjusting the number of HRE copies and/or the number of ODDs.

In addition to the at least one ODD, the chimeric polypeptide comprises at least one polypeptide with anti-tumour properties. Such proteins for delivery to a tumour, include but are not limited to any one or more of the following: immune stimulating antibodies; surface or intracellular receptors that confer cell activation and tumour-killing capability; a T-cell Receptor (TCR), an NK receptor, a Toll-like receptor. Also included are co-receptors that associate with the polypeptide with anti-tumour properties, for example, to facilitate intracellular signalling.

According to one embodiment, the chimeric polypeptide encoded by the nucleic acid comprises or consists of a CAR polypeptide sequence. A further aspect of the present invention provides a CAR, the expression of which is driven by the regulatory nucleic acid sequence according to the first aspect of the invention, and which CAR also comprises one or more ODDs and at least one polypeptide with anti-tumour properties.

An example of a CAR according to the present invention is provided below, with the amino acid sequence (SEQ ID NO: 35) and the corresponding nucleotide sequence (SEQ ID NO: 36) presented, and in which the CSF1-R Leader Seq (including an optional additional glycine) is in bold and underlined; the T1E peptide (derived from human TGFα□ and EGF) is in bold; the CD28 (extracellular, transmembrane and intracellular domains) is in italics; CD3ζ (intracellular domain) is underlined, and the ODD domain (derived from human HIF1-alpha) is grey shaded.

An example of a CAR according to the present invention is provided below, with the amino add sequence (SEQ ID NO: 35) and the corresponding nucleotide sequence (SEQ ID NO: 36) presented, and in which the CSF1-R Leader Seq (including an optional additional glycine) is in bold, lower case; the T1E peptide (derived from human TGFα and EGF) is in bold, upper case; the CD28 (extracellular, transmembrane and intracellular domains) is in italics, upper case; CD3ζ (intracellular domain) is lower case, and the ODD domain (derived from human HIF1-alpha) is in upper case.

T1E28z CAR and fused ODD amino acid
sequence
(SEQ ID NO: 35)
mgpgvIIIIIvatawhgqg(g)VVSHFNDCPLSHI
DGYCLHIDGVCMYIEALDKYACNCVVGYIGERCQY
RDLKWWELRAAAIEVMYPPPYLDNEKSNGTIIHVK
GKHLCPSPLFPGPSKPFWVLVVVGGVLACYSLLVT
VAFIIFWVRSKRSRLLHSDYMNMTPRRPGPTRKHY
QPYAPPRDFAAYRSrvkfsrsadapayqqgqnqly
nelnlgrreeydvIdkrrgrdpemggkprrknpqe
glynelqkdkmaeayseigmkgerrrgkghdglyq
glstatkdtydalhmqalpprAPAAGDTIISLDFG
SNDTETDDQQLEEVPLYNDVMLPSPNEKLQNINLA
MSPLPTAETPKPLRSSADPALNQEVALKLEPNPES
LELSFTMPQIQDQTPSPSDGSTRQSSPEPNSPSEY
CFYVDSDMVNEFKLELVEKLFAEDTEAKNPFSTQD
TDLDLEMLAPYIPMDDDFQLRSFDQLSPLESSSAS
PESASPQSTVTVFQ
Corresponding nucleotide sequence
(SEQ ID NO: 36)
atgggcccaggagttctgctgctcctgctggtggc
cacagcttggcatggtcagggaggtGTGGTGTCGC
ACTTCAATGACTGTCCACTGTCGCACGATGGATAC
TGCCTCCATGATGGTGTGTGCATGTACATCGAGGC
ATTGGACAAGTATGCATGCAACTGTGTCGTCGGCT
ACATCGGAGAGCGATGTCAGTACCGAGACCTGAAG
TGGTGGGAACTGAGAGCGGCCGCAATTGAAGTTAT
GTATCCTCCTCCTTACCTAGACAATGAGAAGAGCA
ATGGAACCATTATCCATGTGAAAGGGAAACACCTT
TGTCCAAGTCCCCTATTTCCCGGACCTTCTAAGCC
CTTTTGGGTGCTGGTGGTGGTTGGTGGAGTCCTGG
CTTGCTATAGCTTGCTAGTAACAGTGGCCTTTATT
ATTTTCTGGGTGAGGAGTAAGAGGAGCAGGCTCCT
GCACAGTGACTACATGAACATGACTCCCCGCCGCC
CCGGGCCCACCCGCAAGCATTACCAGCCCTATGCC
CCACCACGCGACTTCGCAGCCTATCGCTCCagagt
gaagttcagcaggagcgcagacgcccccgcgtacc
agcagggccagaaccagctctataacgagctcaat
ctaggacgaagagaggagtacgatgttttggacaa
gagacgtggccgggaccctgagatggggggaaagc
cgagaaggaagaaccctcaggaaggcctgtacaat
gaactgcagaaagataagatggcggaggcctacag
tgagattgggatgaaaggcgagcgccggaggggca
aggggcacgatggcctttaccagggtctcagtaca
gccaccaaggacacctacgacgcccttcacatgca
ggccctgccccctcgcGCCCCAGCCGCTGGAGACA
CAATCATATCTTTAGATTTTGGCAGCAACGACACA
GAAACTGATGACCAGCAACTTGAGGAAGTACCATT
ATATAATGATGTAATGCTCCCCTCACCCAACGAAA
AATTACAGAATATAAATTTGGCAATGTCTCCATTA
CCCACCGCTGAAACGCCAAAGCCACTTCGAAGTAG
TGCTGACCCTGCACTCAATCAAGAAGTTGCATTAA
AATTAGAACCAAATCCAGAGTCACTGGAACTTTCT
TTTACCATGCCCCAGATTCAGGATCAGACACCTAG
TCCTTCCGATGGAAGCACTAGACAAAGTTCACCTG
AGCCTAATAGTCCCAGTGAATATTGTTTTTATGTG
GATAGTGATATGGTCAATGAATTCAAGTTGGAATT
GGTAGAAAAACTTTTTGCTGAAGACACAGAAGCAA
AGAACCCATTTTCTACTCAGGACACAGATTTAGAC
TTGGAGATGTTAGCTCCCTATATCCCAATGGATGA
TGACTTCCAGTTACGTTCCTTCGATCAGTTGTCAC
CATTAGAAAGCAGTTCCGCAAGCCCTGAAAGCGCA
AGTCCTCAAAGCACAGTTACAGTATTCCAG

Polynucleotides

According to one aspect of the present invention, there is provided a nucleic acid molecule encoding a chimeric polypeptide, which chimeric polypeptide may comprise a CAR.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present polynucleotide may further comprise a nucleic acid sequence encoding a selectable marker. Suitably selectable markers are well known in the art and include, but are not limited to, fluorescent proteins—such as green fluorescent protein (GFP). The nucleic acid sequence encoding a selectable marker may be provided in combination with a nucleic acid sequence encoding the present CAR in the form of a polycistronic nucleic acid construct. Such a nucleic acid construct may be provided in a vector.

The nucleic acid sequences encoding the CAR and the selectable marker may be separated by a co-expression site which enables expression of each polypeptide as a discrete entity. Suitable co-expression sites are known in the art and include, for example, internal ribosome entry sites (IRES) and self-cleaving peptides.

Further suitable co-expression sites/sequences include self-cleaving or cleavage domains. Such sequences may either auto-cleave during protein production or may be cleaved by common enzymes present in the cell. Accordingly, inclusion of such self-cleaving or cleavage domains in the polypeptide sequence enables a first and a second polypeptide to be expressed as a single polypeptide, which is subsequently cleaved to provide discrete, separated functional polypeptides.

The use of a selectable marker is advantageous as it allows a cell in which a polynucleotide or vector of the present invention has been successfully introduced (such that the encoded CAR is expressed) to be selected and isolated from a starting cell population using common methods, e.g. flow cytometry.

Codon Optimisation

The polynucleotides used in the present invention may be codon-optimised. Codon optimisation has previously been described in WO 1999/41397 and WO 2001/79518.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Vectors

A further aspect of the invention provides vectors comprising the polynucleotide sequences of the invention.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Other methods for transfection include DNA, RNA, mRNA, proteins, plasmids, proteins having transposase activity, proteins with the ability to cut DNA (e.g. Cas proteins bound to sgRNAs (small guide RNAs), molecules for editing nucleic acids, such as Cas9 protein alone or linked to guide RNA (gRNA).

Various methods are known in the art for editing nucleic acid, for example to cause gene knockout, knock-in or expression of a gene to be downregulated or overexpressed, or to introduce mutations in the form of one or more deletions, insertions or substitutions. For example, use of various nuclease systems, such as zinc finger nucleases (ZFN), transcription activator-like effector nucleases (TALEN), meganucleases, or combinations thereof are known in the art for editing nucleic acid and may be used in the present invention. In recent times, the clustered regularly interspersed short palindromic repeats (CRISPR)/CRISPR-associated (Cas) (CRISPR/Cas) nuclease system has become more commonly used for genome engineering. The CRISPR/Cas system is detailed in, for example WO2013/176772, WO2014/093635 and WO2014/089290.

For example, a CRISPR/Cas9 may include a guide RNA (gRNA) sequence with a binding site for Cas9 and a targeting sequence specific for the area to be modified. The Cas9 binds the gRNA to form a ribonucleoprotein that binds and cleaves the target area. In addition to the CRISPR/Cas 9 platform (which is a type II CRISPR/Cas system), alternative systems exist including type I CRISPR/Cas systems, type III CRISPR/Cas systems, and type V CRISPR/Cas systems. Any of the above CRISPR systems may be used to prepare vectors comprising the polynucleotide sequences of the invention.

Immunoresponsive Cells

A further aspect the present invention provides an immunoresponsive cell comprising a nucleic acid molecule encoding a chimeric polypeptide comprising one or more Oxygen-Dependent Degradation Domains (ODD) and at least one polypeptide with anti-tumour properties. Multiple nucleic acids can be operably linked to the said hypoxia-responsive regulatory nucleic acid in the form of bicistronic or polycistronic vectors, separated by IRES or self-cleaving 2A peptides. In a further aspect, the present invention provides a CAR comprising one or more Oxygen-Dependent Degradation Domains (ODD) and at least one polypeptide with anti-tumour properties in an immunoresponsive cell.

In one embodiment, the immunoresponsive cells are capable of expressing a nucleic acid encoding a CAR(s). These cells are “engineered cells”, meaning that the cell has been modified to comprise or express a polynucleotide which is not naturally encoded by the cell. Alternatively, an engineered cell may be modified to overexpress a naturally expressed polynucleotide or to reduce/silence natural expression (knock-down with shRNA, for example). Methods for engineering cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

Accordingly, the nucleic acid molecule encoding a CAR as described herein is not naturally expressed by a corresponding, unmodified cell. Suitably, an engineered cell is a cell whose genome has been modified e.g. by transduction or by transfection. Suitably, an engineered cell is a cell whose genome has been modified by retroviral transduction. Suitably, an engineered cell is a cell whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells according to the present invention may be generated by introducing DNA or RNA encoding a CAR as described herein by one of many means including transduction with a viral vector, transfection with DNA or RNA. Cells may be activated and/or expanded prior to, or after, the introduction of a polynucleotide encoding the CAR as described herein. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The nucleic acid molecule encoding a chimeric polypeptide comprising one or more ODDs, and at least one polypeptide with anti-tumour properties, may be comprised in any mammalian cell, preferably an immunoresponsive cell or a tumour cell. The cell may be in vitro or in vivo. The immunoresponsive cell may comprise the chimeric polypeptide, which itself may be comprised in a chimeric antigen receptor (CAR), wherein the CAR is expressed under conditions of hypoxia, with substantially no expression under normoxic conditions.

Suitable immunoresponsive cells include, but are not limited to, lymphoid-derived cell such as Natural Killer cells, NK T-cell, invariant NKT-cell, or T-cell, such as cytotoxic T-cells, helper T-cells or regulatory T-cells; an αβ T-cell, γδ T-cell, B-cell, or myeloid-derived cells such as a macrophages or neutrophils; stem cells, induced pluripotent stem cells (iPSCs).

Suitably, the immunoresponsive cell, such as a T-cell, is isolated from peripheral blood mononuclear cells (PBMCs) obtained from the subject. Suitably the subject is a mammal, preferably a human. The immunoresponsive cell may optionally be allogenic, in the case of an “off the shelf” CAR T-Cell, where the T cells are not necessarily derived from the subject with cancer (see for example Depil et al., 2020 (Nature Reviews Drug Discovery)).

Suitably the cell is matched or is autologous to the subject. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is matched or autologous to the subject.

A further aspect of the invention provides immunoresponsive cells, particularly T-cells, obtainable or obtained by the method of the invention, as well as pharmaceutical compositions comprising the same.

Method for the Preparation of an Immunoresponsive Cell

In a further aspect of the invention, there is provided a method for preparing an immuno-responsive cell, the method comprising
Isolating lymphoid-derived or myeloid-derived cells from a subject (which may be a cancer patient or a healthy donor);
Modifying said cells to introduce a nucleic acid molecule and/or CAR as defined herein;
Expanding said modified cells ex-vivo;
Obtaining cells capable of expressing a nucleic acid molecule and/or CAR under conditions of hypoxia.

Expression of the nucleic acid molecule or CAR is driven by a hypoxia-responsive regulatory nucleic acid comprising a plurality of HREs, as defined herein.

The immunoresponsive cells of the present invention may be generated by introducing DNA or RNA coding for the nucleic acid molecule and/or CAR(s) as defined herein, by one of many means including transduction with a viral vector, transfection with DNA or RNA.

The cell of the invention may be made by: introducing to a cell (e.g. by transduction or transfection) the polynucleotide or vector as defined herein. Suitably, the cell may be from a sample isolated from a subject.

A further aspect of the present invention provides immunoresponsive cells obtainable by the method of the invention, as well as pharmaceutical compositions comprising the same.

Pharmaceutical Composition

A pharmaceutical composition is a composition that comprises, essentially consists of, or consists of a therapeutically effective amount of a pharmaceutically active agent, the pharmaceutically active agent here being a modified immunoresponsive cell. It preferably includes a pharmaceutically acceptable carrier, diluent or excipient (including combinations thereof). Acceptable carriers or diluents for therapeutic use are well known, and are described, for example, in Remington's Pharmaceutical Sciences, Mack Publishing Co., (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Method of Treatment

A further aspect of the present invention provides a method for the treatment of a tumor, comprising administering immunoresponsive cells of the invention to a subject in need thereof.

The subject suitable for treatment as described herein include mammals, such as a human, non-human primate, cow, horse, pig, sheep, goat, dog, cat, rabbit, or rodent. In preferred embodiments, the subject is a human. Practice of methods described herein in other mammalian subjects, especially mammals that are conventionally used as models for demonstrating therapeutic efficacy in humans (e.g. murine, primate, porcine, canine, or rabbit animals), is also encompassed. Standard dose-response studies are used to optimise dosage and dosing schedule.

“Administering” refers to the physical introduction of the immunoresponsive cells to a subject using any of the various known methods and delivery systems. Examples include intratumoral (i.t.), intravenous (i.v.), intramuscular, subcutaneous, intraperitoneal, intrapleural, spinal, pleural effusion, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intracavitary, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

The immunoresponsive cells are useful in therapy or in prophylactic treatment to stimulate a T-cell mediated immune response to a target cell population. The invention further provides a method for stimulating a T-cell mediated immune response to a target cell population in a patient in need thereof, said method comprising administering to the patient a population of immunoresponsive cells as described above.

The immunoresponsive cells are particularly useful in the treatment of solid cancers. In the CAR-based anti-cancer immunotherapy according to the invention, T-lymphocytes are isolated from a cancer patient (or healthy donor), modified and expanded ex-vivo by, for example, retro/lenti-viral vectors to constitutively express a CAR molecule at the cell surface, with binding specificity for a tumour-associated antigen (TAA) expressed on the surface by the tumour cell, and then are re-infused back into the patient (FIG. 1). As a result, a large population of patient autologous T-cells and/or non-patient derived allogenic T-cells is redirected towards killing cancerous cells. Furthermore, the dual oxygen sensing properties of the CAR allows for off target effects to be reduced or eliminated (through the use of the hypoxia responsive promoter in conjunction with the activity of the ODD(s)), and furthermore, there is increased expression of the anti-tumour polypeptide at the site of the tumour due to the unexpectedly increased strength of the hypoxia-responsive promoter compared to conventional constitutive retroviral promoters.

A method for treating a disease relates to the therapeutic use of the immunoresponsive cells of the present invention. In this respect, the cells may be administered to a subject having an existing disease or condition in order to lessen, reduce or improve at least one symptom associated with the disease and/or to slow down, reduce or block the progression of the disease.

The method of treatment may comprise prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method of treatment need not be carried out using T-cells, but may also be carried out using other suitable immunoresponsive cells such as lymphoid-derived cells such as Natural Killer cell, B-cell, invariant NKT-cell or T-cell, such as cytotoxic T-cells, helper T-cells or regulatory T-cells; or myeloid-derived cells such as a macrophages or neutrophils.

The disclosed methods are useful for treating cancer, for example, inhibiting cancer growth, including complete cancer remission, for inhibiting cancer metastasis, and for promoting cancer resistance. The term “cancer growth” generally refers to any one of a number of indices that suggest change within the cancer to a more developed form. Indices for measuring an inhibition of cancer growth include but are not limited to a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of cytolytic T-lymphocytes, and a decrease in levels of tumour-specific antigens. The term “cancer resistance” refers to an improved capacity of a subject to resist cancer growth, in particular growth of a cancer already had. In other words, the term “cancer resistance” refers to a decreased propensity for cancer growth in a subject.

Cancer cells in the individual with cancer may be immunologically distinct from normal somatic cells in the individual. For example, the cancer cells may express an antigen which is not expressed by normal somatic cells in the individual (i.e. a tumour antigen). Tumour antigens are well-known in the art and are described in more detail herein.

Various types of cancers are known in the art. The cancer may be metastatic or non-metastatic. The cancer may be familial or sporadic. In some embodiments, the cancer is selected from the group consisting of: leukaemia and multiple myeloma. Additional cancers that can be treated using the methods of the invention include, for example, benign and malignant solid tumours and benign and malignant non-solid tumours.

For example, a cancer may comprise a solid tumour, for example, a carcinoma or a sarcoma.

Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures.

Carcinomas that may be treated include adrenocortical, acinar, acinic cell, acinous, adenocystic, adenoid cystic, adenoid squamous cell, cancer adenomatosum, adenosquamous, adnexel, cancer of adrenal cortex, adrenocortical, aldosterone-producing, aldosterone-secreting, alveolar, alveolar cell, ameloblastic, ampullary, anaplastic cancer of thyroid gland, apocrine, basal cell, basal cell, alveolar, comedo basal cell, cystic basal cell, morphea-like basal cell, multicentric basal cell, nodulo-ulcerative basal cell, pigmented basal cell, sclerosing basal cell, superficial basal cell, basaloid, basosquamous cell, bile duct, extrahepatic bile duct, intrahepatic bile duct, bronchioalveolar, bronchiolar, bronchioloalveolar, bronchoalveolar, bronchoalveolar cell, bronchogenic, cerebriform, cholangiocelluarl, chorionic, choroids plexus, clear cell, cloacogenic anal, colloid, comedo, corpus, cancer of corpus uteri, cortisol-producing, cribriform, cylindrical, cylindrical cell, duct, ductal, ductal cancer of the prostate, ductal cancer in situ (DCIS), eccrine, embryonal, cancer en cuirasse, endometrial, cancer of endometrium, endometroid, epidermoid, cancer ex mixed tumour, cancer ex pleomorphic adenoma, exophytic, fibrolamellar, cancer fibro sum, follicular cancer of thyroid gland, gastric, gelatinform, gelatinous, giant cell, giant cell cancer of thyroid gland, cancer gigantocellulare, glandular, granulose cell, hepatocellular, Hurthle cell, hypernephroid, infantile embryonal, islet cell carcinoma, inflammatory cancer of the breast, cancer in situ, intraductal, intraepidermal, intraepithelial, juvenile embryonal, Kulchitsky-cell, large cell, leptomeningeal, lobular, infiltrating lobular, invasive lobular, lobular cancer in situ (LCIS), lymphoepithelial, cancer medullare, medullary, medullary cancer of thyroid gland, medullary thyroid, melanotic, meningeal, Merkel cell, metatypical cell, micropapillary, mucinous, cancer muciparum, cancer mucocellulare, mucoepidermoid, cancer mucosum, mucous, nasopharyngeal, neuroendocrine cancer of the skin, noninfiltrating, non-small cell, non-small cell lung cancer (NSCLC), oat cell, cancer ossificans, osteoid, Paget's, papillary, papillary cancer of thyroid gland, periampullary, preinvasive, prickle cell, primary intrasseous, renal cell, scar, schistosomal bladder, Schneiderian, scirrhous, sebaceous, signet-ring cell, cancer simplex, small cell, small cell lung cancer (SCLC), spindle cell, cancer spongiosum, squamous, squamous cell, terminal duct, anaplastic thyroid, follicular thyroid, medullary thyroid, papillary thyroid, trabecular cancer of the skin, transitional cell, tubular, undifferentiated cancer of thyroid gland, uterine corpus, verrucous, villous, cancer villosum, yolk sac, squamous cell particularly of the head and neck, oesophageal squamous cell, and oral cancers and carcinomas.

Another broad category of cancers includes sarcomas and fibrosarcomas, which are tumours whose cells are embedded in a fibrillar or homogeneous substance, such as embryonic connective tissue.

Sarcomas that may be targeted include adipose, alveolar soft part, ameloblastic, avian, botryoid, sarcoma botryoides, chicken, chloromatous, chondroblastic, clear cell sarcoma of kidney, embryonal, endometrial stromal, epithelioid, Ewing's, fascial, fibroblastic, fowl, giant cell, granulocytic, hemangioendothelial, Hodgkin's, idiopathic multiple pigmented hemorrhagic, immunoblastic sarcoma of B cells, immunoblastic sarcoma of T-cells, Jensen's, Kaposi's, Kupffer cell, leukocytic, lymphatic, melanotic, mixed cell, multiple, lymphangio, idiopathic haemorrhagic, multipotential primary sarcoma of bone, osteoblastic, osteogenic, parosteal, polymorphous, pseudo-Kaposi, reticulum cell, reticulum cell sarcoma of the brain, rhabdomyosarcoma, Rous, soft tissue, spindle cell, synovial, telangiectatic, sarcoma (osteosarcoma)/malignant fibrous histiocytoma of bone, and soft tissue sarcomas.

Lymphomas that may be treated include Acquired Immune Deficiency Syndrome (AIDS)-related, non-Hodgkin's, Hodgkin's, T-cell, T-cell leukaemia/lymphoma, African, B-cell, B-cell monocytoid, bovine malignant, Burkitt's, centrocytic, lymphoma cutis, diffuse, diffuse, large cell, diffuse, mixed small and large cell, diffuse, small cleaved cell, follicular, follicular centre cell, follicular, mixed small cleaved and large cell, follicular, predominantly large cell, follicular, predominantly small cleaved cell, giant follicle, giant follicular, granulomatous, histiocytic, large cell, immunoblastic, large cleaved cell, large non-cleaved cell, Lennert's, lymphoblastic, lymphocytic, intermediate; lymphocytic, intermediately differentiated, plasmacytoid; poorly differentiated lymphocytic, small lymphocytic, well differentiated lymphocytic, lymphoma of cattle; Mucosa-Associated Lymphoid Tissue (MALT), mantle cell, mantle zone, marginal zone, Mediterranean lymphoma, mixed lymphocytic-histiocytic, nodular, plasmacytoid, pleomorphic, primary central nervous system, primary effusion, small B-cell, small cleaved cell, small non-cleaved cell, T-cell lymphomas; convoluted T-cell, cutaneous T-cell, small lymphocytic T-cell, undefined lymphoma, u-cell, undifferentiated, aids-related, central nervous system, cutaneous T-cell, effusion (body cavity based), thymic lymphoma, and cutaneous T-cell lymphomas.

Leukaemias and other blood cell malignancies that may be targeted include acute lymphoblastic, acute myeloid, acute lymphocytic, acute myelogenous leukaemia, chronic myelogenous, hairy cell, erythroleukaemia, lymphoblastic, myeloid, lymphocytic, myelogenous, leukaemia, hairy cell, T-cell, monocytic, myeloblastic, granulocytic, gross, hand mirror-cell, basophilic, haemoblastic, histiocytic, leukopenic, lymphatic, Schilling's, stem cell, myelomonocytic, monocytic, prolymphocytic, promyelocytic, micromyeloblastic, megakaryoblastic, megakaryoctyic, Rieder cell, bovine, aleukemic, mast cell, myelocytic, plasma cell, subleukaemic, multiple myeloma, nonlymphocytic, chronic myelogenous leukaemia, chronic lymphocytic leukaemia, polycythemia vera, lymphoma, Hodgkin's disease, non-Hodgkin's lymphoma (indolent and high grade forms), multiple myeloma, Waldenstrom's macroglobulinaemia, heavy chain disease, myelodysplastic syndrome, myelodysplasia and chronic myelocytic leukaemias.

Brain and central nervous system (CNS) cancers and tumours that may be treated include astrocytomas (including cerebellar and cerebral), brain stem glioma, brain tumours, malignant gliomas, ependymoma, glioblastoma, medulloblastoma, supratentorial primitive neuroectodermal tumours, visual pathway and hypothalamic gliomas, primary central nervous system lymphoma, ependymoma, brain stem glioma, visual pathway and hypothalamic glioma, extracranial germ cell tumour, medulloblastoma, myelodysplastic syndromes, oligodendroglioma, myelodysplastic/myeloproliferative diseases, myelogenous leukaemia, myeloid leukaemia, multiple myeloma, myeloproliferative disorders, neuroblastoma, plasma cell neoplasm/multiple myeloma, central nervous system lymphoma, intrinsic brain tumours, astrocytic brain tumours, gliomas, and metastatic tumour cell invasion in the central nervous system.

Gastrointestinal cancers that may be treated include extrahepatic bile duct cancer, colon cancer, colon and rectum cancer, colorectal cancer, gallbladder cancer, gastric (stomach) cancer, gastrointestinal carcinoid tumour, gastrointestinal carcinoid tumours, gastrointestinal stromal tumours, bladder cancers, islet cell carcinoma (endocrine pancreas), pancreatic cancer, islet cell pancreatic cancer, prostate cancer rectal cancer, salivary gland cancer, small intestine cancer, colon cancer, and polyps associated with colorectal neoplasia.

Lung and respiratory cancers that may be treated include bronchial adenomas/carcinoids, oesophageal cancer, hypopharyngeal cancer, laryngeal cancer, hypopharyngeal cancer, lung carcinoid tumour, non-small cell lung cancer, small cell lung cancer, small cell carcinoma of the lungs, mesothelioma, nasal cavity and paranasal sinus cancer, nasopharyngeal cancer, nasopharyngeal cancer, oral cancer, oral cavity and lip cancer, oropharyngeal cancer; paranasal sinus and nasal cavity cancer, and pleuropulmonary blastoma.

Urinary tract and reproductive cancers that may be treated include cervical cancer, endometrial cancer, ovarian epithelial cancer, extragonadal germ cell tumour, extracranial germ cell tumour, extragonadal germ cell tumour, ovarian germ cell tumour, gestational trophoblastic tumour, spleen, kidney cancer, ovarian cancer, ovarian epithelial cancer, high grade serous ovarian cancer, ovarian germ cell tumour, ovarian low malignant potential tumour, penile cancer, renal cell cancer (including carcinomas), renal cell cancer, renal pelvis and ureter (transitional cell cancer), transitional cell cancer of the renal pelvis and ureter, gestational trophoblastic tumour, testicular cancer, ureter and renal pelvis, transitional cell cancer, urethral cancer, endometrial uterine cancer, uterine sarcoma, vaginal cancer, vulvar cancer, ovarian carcinoma, primary peritoneal epithelial neoplasms, cervical carcinoma, uterine cancer and solid tumours in the ovarian follicle), superficial bladder tumours, invasive transitional cell carcinoma of the bladder, and muscle-invasive bladder cancer.

Skin cancers and melanomas (as well as non-melanomas) that may be treated include cutaneous T-cell lymphoma, intraocular melanoma, tumour progression of human skin keratinocytes, basal cell carcinoma, and squamous cell cancer. Liver cancers that may be targeted include extrahepatic bile duct cancer, and hepatocellular cancers. Eye cancers that may be targeted include intraocular melanoma, retinoblastoma, and intraocular melanoma.

Hormonal cancers that may be treated include: parathyroid cancer, pineal and supratentorial primitive neuroectodermal tumours, pituitary tumour, thymoma and thymic carcinoma, thymoma, thymus cancer, thyroid cancer, cancer of the adrenal cortex, and adrenocorticotrophic hormone (ACTH)-producing tumours.

Miscellaneous other cancers that may be targeted include advanced cancers, AIDS-related, anal cancer adrenal cortical, aplastic anaemia, aniline-induced and betel-induced cancers, buyo cheek cancer, cerebriform, chimney-sweeps' carcinoma, clay pipe-induced cancer, colloid cancer, cystic, dendritic, cancer a deux, duct, dye workers, encephaloid, cancer en cuirasse, endometrial, endothelial, epithelial, glandular, cancer in situ, Kang cancer, Kangri cancer, latent, medullary, melanotic, mule-spinners', occult cancer, paraffin, pitch workers', scar, schistosomal bladder, scirrhous, lymph node, soft, soot, spindle cell, swamp, tar, and tubular cancers.

Miscellaneous other cancers that may be targeted also include carcinoid (gastrointestinal and bronchial), Castleman's disease, chronic myeloproliferative disorders, clear cell sarcoma of tendon sheaths, Ewing's family of tumours, head and neck cancer, lip and oral cavity cancer, metastatic squamous neck cancer with occult primary, multiple endocrine neoplasia syndrome, tumour, mycosis fungoides, pheochromocytoma, Sezary syndrome, supratentorial primitive neuroectodermal tumours, tumours of unknown primary site, peritoneal effusion, malignant pleural effusion, trophoblastic neoplasms, and hemangiopericytoma.

The cancer may particularly include but is not limited to any of the following: lung, breast, ovarian, head and neck, pancreatic, epithelioma, sarcoma, neuroblastoma, prostate, colorectal, gastric, small intestine, hepatic, bone, testicular, renal, thyroid cancers.

Method for Determining a Subject's Suitability for Treatment

A further aspect of the present invention provides a method for determining a subject's suitability for treatment with immunoresponsive cells of the invention. The method may comprise monitoring for the co-expression of at least two, three, four or all five of the following genes: PGK1, SLC2A1, CA9, ALDOA and VEGFA, wherein co-expression of said genes in said subject is indicative of the subject's suitability for treatment. Expression levels of the aforementioned genes may be increased or changed compared to gene expression levels in healthy controls.

Additionally or alternatively, a subject's suitability for treatment with immunoresponsive cells of the invention may be determined by immunohistochemically staining biopsy tissue from a subject and assessing HIF stabilisation in the tumour or stroma and/or monitoring T cell (and/or other immunoresponsive cells) infiltration to HIF stabilised regions of the tumour. Infiltration of the immunoresponsive cells to HIF stabilised regions of the tumour is indicative of a subject's suitability for treatment with the immunoresponsive cells of the invention comprising the HypoxiCAR system.

Kits

A further aspect of the invention provides a kit comprising any one or more of: polypeptides, nucleic acids, constructs, vectors, CARs, immunoresponsive cells and/or a pharmaceutical composition of the invention.

Nucleic acids, polypeptides, CAR constructs, CAR vectors may be combined in a kit, which is supplied with a view to generating immunoresponsive cells of the invention in situ.

Uses

A further aspect of the invention provides use of immunoresponsive cells according to the invention or a pharmaceutical composition comprising the same in the treatment of cancer, particularly a solid cancer.

Also provided is the use of a polypeptide, nucleic acids, constructs, vectors, CARs and immunoresponsive cells according to the invention, or use of a pharmaceutical composition comprising the same in the treatment of cancer, particularly a solid cancer.

The invention also provides use of the regulatory nucleic acids of the invention for driving increased expression of a CAR under hypoxic conditions compared to the corresponding non-modified wild type counterpart under the same conditions. The use of the hypoxia-responsive regulatory sequence of the invention is particularly advantageous when targeting in transient or low-level hypoxia, when targeting low-density antigens and when using a weak therapeutic agent, such as a weak CAR.

Also provided is the use of a hypoxia-responsive regulatory nucleic acid according to the first aspect of the invention in the prevention or reduction of tonic CAR signalling. Also provided is the use of the dual sensing system of the present invention (i.e. the use of a hypoxia-responsive regulatory nucleic acid in conjunction with the use of one or more ODDs) in the prevention of tonic CAR signalling.

Advantageously, tonic CAR signalling is substantially prevented or reduced through the dual sensing system of the invention. Tonic antigen-independent signalling in CAR T-cells, both during their ex vivo expansion and following their in vivo infusion, can increase differentiation and exhaustion of T-cells leading to decreased potency in vivo. This basal tonic signalling is commonly present due to the high cell surface density and self-aggregating properties of CARs. Advantageously, in the methods of the invention, the immunoresponsive cells, which contain the CAR-coding DNA, does not express any (or expresses only a minimal number of) CARs on its cell surface, unless in a hypoxic environment, i.e. a solid tumour. When this CAR T-cell is found in areas of hypoxia or in the tumour microenvironment, it will express CARs on its surface at high density that will cause sustained T-cell activation and T-cell mediated tumour killing, should the antigen target be present.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows a schematic representation of CAR T-cell immunotherapy. In the practice of CAR T-cell immunotherapy, T-cells are isolated from the cancer patient and genetically modified ex-vivo, for example using retro- or lentiviral particles or RNA electroporation. By this means, the T-cells are engineered to express a chimeric receptor (CAR) with specific binding affinity to a tumour antigen of interest. Following this genetic modification, the resultant CAR-expressing T-cells are expanded using appropriate cytokines and the expanded population is re-infused back into the patient leading to T-cell-mediated targeting of the cancer.

FIG. 2 shows a schematic representation of oxygen sensing in the mammalian cell. Under conditions of normoxia (left), HIF1α is hydroxylated by PHD enzymes in a process that requires oxygen. Hydroxylated HIF1α is then able to bind to pVHL ubiquitin ligases, which add ubiquitin on the HIF1α molecule causing its proteasomal degradation. Under conditions of hypoxia (right), due to the lack of oxygen, HIF1α hydroxylation and degradation is blocked leading to the stabilisation of the HIF1α. Stabilised HIF1α then translocates to the nucleus, where it forms a complex with HIF1β and other molecules (such as P300 and CBP). This complex is then able to bind to HIF-binding sites (HREs) that are present upstream of hypoxia-inducible genes and activates their transcription.

FIG. 3 shows a schematic representation of the system of the present invention in which a cytotoxic T-lymphocyte (CTL), which when in the circulation or in tissue under normal oxygen tension, will not express on its surface any artificial receptor. However, when it is located in a hypoxic region, the CTL will express a cell surface CAR that will have specific binding affinity for a cancer antigen of interest. Therefore, CTL-mediated killing will happen only when both hypoxia and the antigen of interest are present, owing to the presence of the hypoxia-responsive regulatory nucleic acid.

FIG. 4 shows the frequency logos of nucleotides in HIF-binding or ancillary sites: A. Frequency of HIF-binding nucleotides in human hypoxia-inducible genes B. Frequency of HIF-binding nucleotides in mouse hypoxia-inducible genes C. Frequency of HIF-ancillary nucleotides in hypoxia-inducible genes. The height of each letter is representative of the frequency of occurrence of the corresponding nucleotide in each position.

FIG. 5 shows an example of a 3 tandem HRE design. The human erythropoietin (hEPO) HRE includes 3 HREs in tandem, wherein each single HRE includes HIF-binding-linker-HIF-ancillary sequences derived from the human EPO gene. The human vascular endothelial growth factor A (hVEGFA) HRE includes 3 HREs in tandem, wherein each single HRE includes HIF-binding-linker-HIF-ancillary sequences derived from the human VEGFA gene. The human glucose transporter 3(hGLUT3) HRE includes 3 HREs in tandem, wherein each single HRE includes HIF-binding-linker-HIF-ancillary sequences derived from the human GLUT3 gene.

FIG. 6 shows a linear map representation of constructs used to optimise the technology: A. The long terminal repeat (LTR) unmodified SFG reporter retroviral construct containing click beetle luciferase (cbluc) and enhanced green fluorescent protein (eGFP) cDNAs (reporter SFG), B. A modified reporter SFG vector in which the hEPO HRE has been inserted within the 3′ LTR, C. A modified reporter SFG vector in which the hVEGF HRE has been inserted within the 3′ LTR, D. A modified reporter SFG vector in which the hGLUT3 HRE has been inserted within the 3′ LTR.

FIG. 7 shows the HIF1α amino acid sequence (UniProt database).

FIG. 8 shows a linear map representation of further constructs used to optimise the technology: A. Reporter SFG vector containing cbluc luciferase-ODD fusion, B. Reporter SFG vector containing cbluc luciferase-ODD fusion and hEPO HRE LTR modification, C. Reporter SFG vector containing cbluc luciferase-ODD fusion and hVEGFA HRE LTR modification, D. Reporter SFG vector containing cbluc luciferase-ODD fusion and hGLUT3 HRE LTR modification.

FIG. 9 shows Western blot results. These results represent HIF1α protein levels (and β-Actin reference) detected in cell lines (293T, HT1080, T47D and Jurkat) following their incubation in 0.1% oxygen and 20% oxygen. Bar chart depicts the intensity of HIF1α bands. This was calculated by plotting the bands and calculating the area under the curve (AUC) using ImageJ.

FIG. 10 shows the gating strategy and determination of transduction efficiency (of the unmodified SFG reporter construct) by measuring eGFP fluorescence signal of transduced cells (FIGS. 6 and 8). 7-AAD negative cells (viable cells) are gated and used in evaluating eGFP fluorescence in the histogram.

FIG. 11 shows qPCR assay validation. The graph shows the linear (y=x) relationship between thermal cycle number and the DNA amount (Log scale) in nanograms for detecting genomic TATA-box binding protein gene (TBP) and luciferase (luc; encoded by the constructs) in the transduced cells.

FIG. 12 shows relative light unit (RLU) data obtained from 293T cells following 18 hours of 5% (A), 1% (B) and 0.1% (C) oxygen (right bars) incubation compared to their respective normoxic condition (left bars) for the indicated constructs.

FIG. 13 shows relative light unit (RLU) data obtained following culture of 293T cells for 18 hours in 100 or 0 μM cobalt chloride for the indicated constructs.

FIG. 14 shows mRNA expression of ErbB receptor (egfr and erbb2-4) and integrin β6 (intgb6) genes in healthy mouse tissue. In total, 13 tissues were analysed in this experiment. Tissues are ranked according to their expression level of each mRNA relative to the house keeping gene, Tbp.

FIG. 15 shows the effect of 3 and 9 HRE copies versus the control (constitutive) in the expression of luciferase under conditions of normoxia. The inclusion of the HREs significantly silenced the expression of the downstream reporter transgene (luciferase). NT: non-transduced; Constitutive: wild-type non-HRE modified LTR; 3HRE: LTR modified to contain 3 tandem HRE elements; 9HRE: LTR modified to contain 9 tandem HRE elements. The HRE elements were derived from human EPO gene promoter. By modifying the LTRs (retroviral promoter) to contain multiple HREs, the expression of luciferase was significantly reduced under conditions of normoxia.

FIG. 16 shows the fold induction of luciferase expression under conditions of hypoxia (calculated by dividing gene expression under conditions of hypoxia with that observed under conditions of normoxia). Constitutive: wild-type non-HRE modified LTR; 3HRE: LTR modified to contain 3 tandem HRE elements; 9HRE: LTR modified to contain 9 tandem HRE elements. Under hypoxic conditions (0.1% O₂), the expression of luciferase correlates with the number HREs included in the promoter.

FIG. 17 shows the effect of fusing different lengths of the human HIF1α ODD (amino acid numbers are indicated) onto the C-terminus of click beetle luciferase in SFG vectors containing an unmodified LTR. Gene expression was assessed in normoxic conditions. Constitutive: no ODD addition vs fusion of different indicated lengths of ODD to luciferase.

17A: Constructs containing variable ODDs fused on the C-terminus of Click Beetle luciferase.

17B: T47D cells transduced with constructs shown in A, non-transduced (NT) or constitutive transduced (wild type non ODD modified Click Beetle luciferase) were exposed in hypoxia (0.1% oxygen) for 18 h. Fold induction is the luciferase expression induction seen in hypoxia in relative to the normoxic expression in each construct. N=3 Line=mean and error bars SEM.

FIG. 18 shows the combination of the 9 HRE promoter architecture with the human HIF1α ODD (amino acids 401-603) fused onto the C-terminus of luciferase. This dual oxygen sensing system showed no detectable expression of luciferase under conditions of normoxia, but was switched on in hypoxic conditions (0.1% oxygen).

FIG. 19 shows that T4-CAR T-cells reside in the liver and lung acutely after i.v. infusion. T4-CAR T-cells co-expressing a luciferase reporter were injected i.v. into NSG immunocompromised mice bearing an established subcutaneous SKOV3 tumour. CAR T-cells were tracked using an IVIS bioluminescence imager.

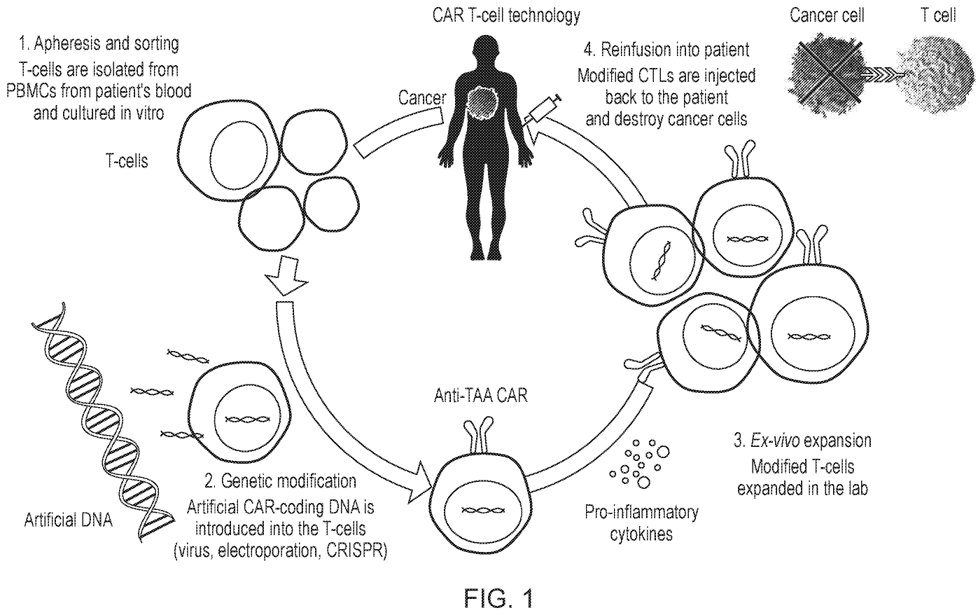

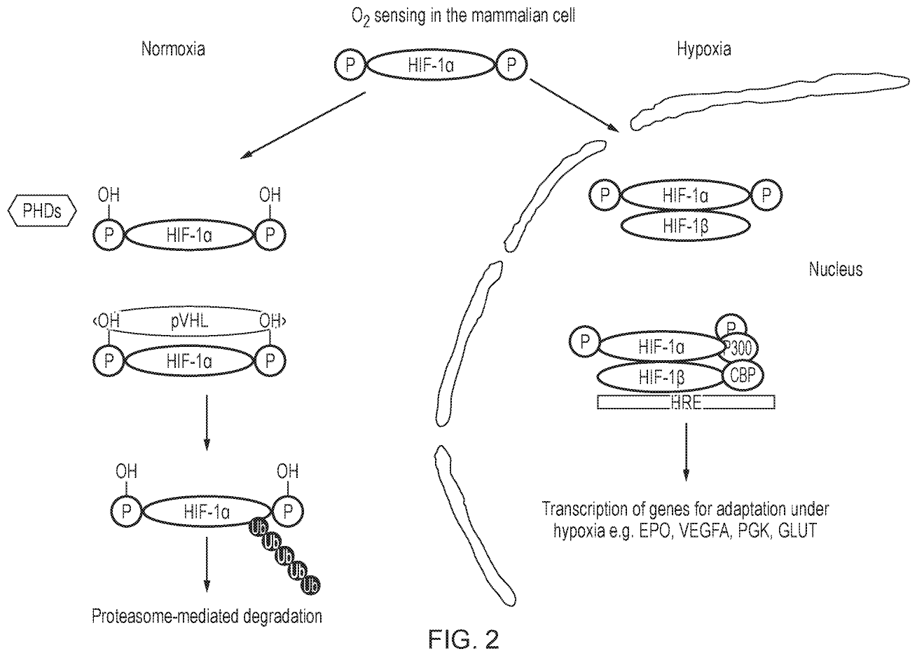

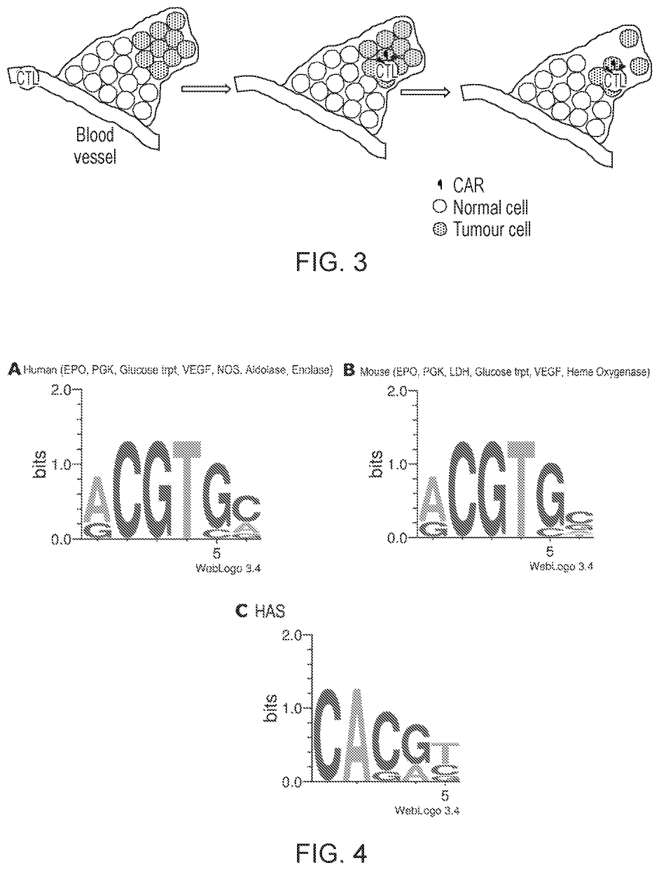

(a) Shows the detected light (shown in blue/green on the picture) from the luciferase that is expressed within the T4-CAR T-cells in three mice bearing established SKOV3 human ovarian tumours implanted subcutaneously (left) and the dissected organs/tumour from a representative mouse (right), 4 days post infusion.

(b) Quantitation of the luciferase signal in each indicated organ (n=6 individual mice). As can be seen at the 4 day timepoint post infusion, these cells preferentially reside in the lung and liver rather than the tumour.

(c) T4-CAR T-cells have specificity for 8 homo- and heterodimers formed by the Erbb receptor family, which are expressed by most, if not all, epithelial cells. Analysis of the vital organs for mRNA expression of the Erbb family (presented relative to the housekeeping gene Tbp), demonstrated that both the lung and liver, where T4-CAR T-cells initially accumulate, are both rich sources of the CAR ligands. N=6 (biological replicates combined).

FIG. 20 shows the median fluorescence intensity (MFI) of CAR expression on the gated detectable CAR T-cells in the indicated groups at 20 h post exposure to 0.1% oxygen (e.g. hypoxic conditions; n=3 individual CAR preparations). ‘T4’ is expressed using the standard SFG vector (LTR-based retroviral promoter) and ‘HRE-CAR’ is expressed using a modified SFG vector (9×HRE elements inserted into the LTR of the SFG vector). The encoded HRE CAR does not contain an additional ODD. Unexpectedly, the median fluorescence intensity (MFI) of CAR expression was greater in the HRE-CAR group.

FIG. 21 shows that HypoxiCAR T-cell effector function is stringently restricted to hypoxic conditions: (a) Schematic diagram depicting the CAR constructs (with 9×HREs in tandem, not shown), and their modular arrangements (when integrated in the genome) that were transduced into human T-cells; LTR-Long terminal repeat. (b) Representative flow cytometry dot plots evaluating surface CAR and CD8α (to identify CD8⁺ T-cells). Data demonstrates CAR expression by live (7AAD⁻) CD3⁺ T4-CAR, HypoxiCAR or non-transduced T-cells that had been maintained in normoxic or hypoxic (0.1% O₂) conditions for 18 h prior to staining and flow cytometry analysis. (c-h) Healthy donor CD3⁺ T-cells (n=6) were transduced to generate T4-CAR or HypoxiCAR T-cells and (c) placed into 0.1% O₂ hypoxic conditions for up to 18 h prior to being transferred back to normoxic conditions, where CAR expression was evaluated at the indicated times using flow cytometry analysis. (d) The median fluorescence intensity (MFI) of CAR expression on T4-CAR and HypoxiCAR T-cells at 18 h of exposure to 0.1% O₂ hypoxia from panel (c). (e) Detectable surface CAR expression on HypoxiCAR T-cells after 18 h exposure to decreasing concentrations of O₂; statistical significance was evaluated in comparison to expression under normoxic conditions. (f) In vitro SKOV3 tumour cell killing by T4 CAR, HypoxiCAR, or CD3-truncated HypoxiCAR (CD3□ endodomain removed to prevent intracellular signalling) T-cells at the indicated times in normoxic and 0.1% O₂ hypoxic conditions. Quantification of IL-2 (g) and IFN-γ (h) released from the T-cells used in (f). ELISA analysis was performed on media collected 72 h post exposure to SKOV3 cells, making comparison with co-cultures performed using untransduced T-cells (“T-cells”). All statistical comparisons that were conducted are shown. Bar on the bar charts shows the group mean and each dot represents an individual healthy donor in the group. * P<0.05, ** P<0.01, *** P<0.001, **** P<0.0001.

FIG. 22 shows in panel A) a schematic of the HypoxiCAR retroviral construct when integrated into the genome of the T-cells. HypoxiCAR T-cells were injected either i.v. or i.t. into HN3 tumour bearing NSG mice. B) 24 hours after infusion, tumours were excised, enzyme-digested and stained for markers of interest prior to flow cytometry analyses. Shown are gated HypoxiCAR T-cells (CD3⁺ and CD45⁺) residing in the indicated tissues, which were assessed for cell surface CAR expression (x axis of histogram). CAR expression was only detected in T-cells residing in the tumour. C) Quantification of surface CAR expression (as seen in B) where each dot represents an individual mouse for each respective tissue.

FIG. 23 shows that HypoxiCAR provides tumour-selective CAR expression in SKOV3 and LL2 tumours: (a) Growth curve of SKOV3 tumours grown in NSG mice (n=6 mice). (b) Representative stacked histograms showing detectable cell surface HypoxiCAR expression in enzyme-dispersed tissues and blood of a SKOV3 tumour bearing mouse that had been injected i.v. and i.t. with HypoxiCAR T-cells 24 h prior to sacrifice. Histograms show gated live (7AAD⁻) Ter119⁻ CD45⁺ CD3⁺ T-cells alongside a CAR isotype stained tumour (grey histogram) (left) and full cohort quantification of percent T-cells with detectable CAR in the respective tissues (across n=6 individual mice). (c) Equivalent experiment to that described in b, but with LL2 tumour bearing Rag2^{-/-} mice, showing representative cell surface HypoxiCAR expression by T-cells within the respective tissues (left) and quantification of the percent CAR expressing HypoxiCAR T-cells (right) in the respective tissues (across n=3 individual mice). Bar on the bar charts shows the group mean and each dot represents an individual healthy mouse in the group. * P<0.05, ** P<0.01, *** P<0.001, **** P<0.0001.

FIG. 24: T4-CAR T-cells cause inflammation in healthy organs. (A) Diagram depicting T4-CAR. (B) Representative histogram showing cell surface CAR expression on live (7AAD⁻) CD3⁺ T4-CAR or non-transduced human T-cells, assessed using flow cytometry. (C-E) Day 13 post subcutaneous HN3 tumour cell inoculation, mice were infused i.v. with vehicle or 10×10⁶ non-transduced or T4-CAR T-cells (n=5). (C) Schematic diagram depicting the experiment. (D) Weight change of the mice. Arrow denotes T-cell infusion; cross indicates an animal that was culled because a humane endpoint had been exceeded. (E) Serum cytokines 24 h post-infusion. (F) Low-dose human ErbB-CAR/Luc T-cells (4.5×10⁶) were infused i.v. into SKOV3 tumour bearing NSG mice and 4 days later, bioluminescence imaging was performed on the whole body and dissected organs. (G) Quantification of the photons/s/unit area as percent of all organs (n=6), LN-inguinal lymph node, SI-small intestine. (H,I) H&E stained sections (left) and quantitation of myeloid infiltration (right) in the lung (H) and liver (I) 5 days post infusion i.v. of low-dose (4.5×10⁶ cells) T4-CAR or untransduced T-cells or vehicle. Arrows indicated myeloid infiltrates. (J,K) Immunohistochemistry (IHC) staining of tissue sections for reductively-activated pimonidazole in tumour bearing NSG mice (J) and quantitation of the staining (K). All experiments are representative of a biological repeat. Line charts, the dots mark mean and error bars represent s.e.m. Bar charts show mean and dots individual mice. * P<0.05, ** P<0.01.

FIG. 25: HypoxiCAR T-cell effector function is stringently restricted to hypoxia. (A) Diagram depicting HypoxiCAR under conditions of normoxia and hypoxia. (B) Representative histograms to show cell surface CAR expression on live (7AAD⁻) CD3⁺ T4-CAR, HypoxiCAR and non-transduced human T-cells in normoxic or 18 h hypoxic (0.1% O₂) conditions, assessed using flow cytometry. (C) Surface CAR expression on HypoxiCAR T-cells at the indicated times under conditions of hypoxia (0.1% O₂) or normoxia assessed using flow cytometry analysis. Values were normalized to those seen at 18 h hypoxia (n=6). (D) Surface CAR expression on HypoxiCAR T-cells after 18 h exposure to 0.1, 1, 5%, 20% O₂ (n=6). Values were normalized to those seen in 0.1% O₂. (E-G) In vitro SKOV3 tumour cell killing by T4-CAR, HypoxiCAR, CD3□-truncated HypoxiCAR (CD3⁻; to prevent intracellular signalling) and non-transduced T-cells (CAR⁺ effector to target tumour cell ratio 1:1) in normoxic and 0.1% O₂ hypoxic conditions. (F) Quantification of IL-2 and (G) IFNγ released into the media from the respective T-cells after 24 h and 48 h exposure to SKOV3 cells respectively, under normoxic and 0.1% O₂ hypoxic conditions. Bar on charts shows mean and dots represent each individual healthy donor. Datapoints were collected in parallel and are representative of a biological repeat. In line charts, the dots mark mean and error bars represent s.e.m. * P<0.05, ** P<0.01, *** P<0.001, **** P<0.0001.

FIG. 26: HypoxiCAR T-cells provide anti-tumour efficacy without systemic toxicity. (A-C) Subcutaneous HN3 tumour-bearing NSG mice were injected both i.v. and i.t. with human HypoxiCAR T-cells (2.5×10⁵ cells i.t. and 7.5×10⁵ cells i.v.) 72 h prior to sacrifice. (A) Schematic diagram depicting the experiment. (B) Representative histograms showing surface CAR expression on live nucleated cells (7AAD⁻, Ter119⁻), CD45⁺ CD3⁺ HypoxiCAR T-cells in the indicated enzyme-dispersed tissues and blood and (C) quantification in the respective tissues across n=9 individual mice. (D-F) Sixteen days post subcutaneous HN3 tumour cell inoculation, mice were infused i.v. with either vehicle or 10×10⁶ T4-CAR, HypoxiCAR or non-transduced human T-cells (control) (n=4 mice). (D) Schematic diagram depicting the experiment. (E) Weight change of the mice. (F) Serum cytokines 24 h post-infusion. (G,H) low dose (4.5×10⁶) T4-CAR or HypoxiCAR T-cells were infused i.v. into NSG mice. Five days later the indicated tissues were excised, and myeloid infiltration was scored in the lung (G) and liver (H). (I) HN3 tumour growth curves from (D-F), arrow marking the point of CAR T-cell infusion. All experiments are representative of biological repeat. Bar charts shows the mean and each dot an individual mouse. In line charts, the dots marks the mean and error bars represent s.e.m. * P<0.05, ** P<0.01, *** P<0.001, **** P<0.0001.

FIG. 27: T-cells are not excluded from HIF1α stabilized regions of hypoxic squamous cell carcinomas of head and neck (SCCHN)s. (A-C) An HRE-regulated gene signature was constructed from known HRE-regulated genes in SCCHN tumours (n=528). (A) Heatmap displaying the Pearson correlation coefficient for the individual genes. (B) Signature expression based on tumour (T) stage (T1 n=48, T2 n=136, T3 n=99, T4 n=174). (C) Survival curve for patients with Stage 3 and 4 SCCHN for high and low expression of the HRE-regulated gene signature (n=87 respectively). (D) Representative IHC stained SCCHN section for HIF1α (red) and CD3 (brown) (n=60). (E-F) Abundance of intra-epithelial T-cells (IETs) in SCCHN tumours was grouped as low/absent (n=40) and high (n=55). An example of an IET is marked by a black arrow in (D). IET number was assessed against the HIF1α stabilization (H)-score of the tumour (E). For tumours in which high numbers of IETs were present, tumour infiltrating lymphocytes directly infiltrating HIF-1α stabilized regions of the tumour (H-TILs) were grouped as absent (n=6 of 55 tumours) or present (n=46 of 55 tumours). Examples of H-TILs are marked by white arrows in (D). H-IET number was assessed against the H-score of the tumour (F). (G) Confocal images of an oral tongue carcinoma stained with DAPI (nuclei; blue) and antibodies against CD3 (green) and HIF1α (red); white denotes CD3 and HIF1α co-localization. Box plots show median and upper/lower quartiles, whiskers show highest and lowest value. * P<0.05, ** P<0.01, *** P<0.001, **** P<0.0001.

FIG. 28: HypoxiCAR T-cells provide anti-tumor efficacy against established SKOV3 tumours. Human HypoxiCAR T-cells (10×10⁶ i.v.) or non transduced control T-cells were injected i.v. into NSG mice bearing established subcutaneous SKOV3 tumours. Chart shows the growth curves of the respective cohorts of mice. The arrow marking the point of CAR T-cell infusion. The dots mark the mean and error bars s.e.m.

FIG. 29: T4 (constitutive non-HRE-modified) or HRE-modified (HRE alone, lacking ODD) CAR T-cells were cultured for 24 h with SCOV3 target cell lines at the indicated CAR+ effector to target ratios in normoxic (20% oxygen) or hypoxic conditions (0.1% oxygen). A. Shows the % of viable targets in the co-cultures following the 24 h co-culture and B. Shows the IL-2 released in the co-cultures following antigen-specific stimulation of T-cells by the targets. Data shown are means from n=4 independent experiments using T-cells from 4 independent donors for panel A, and means from n=3 independent experiments using T-cells from 3 independent donors for panel B. Error bars show SEM.

### EXAMPLES

The invention will now be described with reference to the following examples.

Materials and Methods

Constructs

Three HRE sequences, each containing three in tandem HBS from human EPO, VEGFA and GLUT3, were synthesized by GeneArt (ThermoFisher Scientific) and flanked by a NheI and an XbaI restriction sites. These sequences were sub-cloned and replaced the natural NheI/XhoI sequence within the 3′ LTR of the SFG Moloney murine leukemia virus plasmid. Specific modification of the 3′ LTR was achieved by the synthesis of a XhoI/EcoRI-flanked intermediate fragment, which contained the HREs, achieved using primers that contained the restriction enzyme sites and complementary sequences to the respective HRE cassettes. Overlapping PCR and sub-cloning of the fragment achieved insertion into the SFG vector. Next, a protein-coding sequence coding for green-emitting variant of click beetle luciferase and green fluorescent protein separated by a P2A was cloned into NcoI/XhoI site of the SFG. Restriction digestions were performed at 37° C. using enzymes and buffers purchased from New England Biolab. DNA was detected in ethidium bromide stained 1.2% agarose gels and bands of appropriate sizes as assessed according to the DNA ladder were excised and extracted from gels using QIAquick Gel Extraction Kit (Qiagen). Sticky end ligations were catalysed by T4 DNA ligase (ThermoFisher Scientific) at 16° C. for 1 hour.

CAR/Reporter Construct Cloning

Human T1E CAR containing SFG retroviral vector was modified to generate the constructs utilized in this study. The full-length ODD cDNA encoding amino acids 401-603 (SEQ ID NO: 29) from human HIF1α was synthesis as a gBlock® (Integrated DNA Technologies) and was appended onto the C-terminus of the CD3ζ within the T1E CAR through overlap PCR using Platinum Pfx DNA polymerase (Thermo Fisher Scientific) according to the manufacturer's instructions with the primers; 5′-TCCAGCGGCTGGGGCGCGAGGGGGCAGGGCC-3′ (SEQ ID NO: 38) and 5′-GGCCCTGCCCCCTCGCGCCCCAGCCGCTGGA-3′ (SEQ ID NO: 39). PCR products were run on 1.2% Agarose (Sigma-Aldrich) gels and product size was estimated against a 1 kb Plus DNA ladder (Thermo Fisher Scientific). Fragments of the expected size were excised and purified using the QIAquick® Gel Extraction kit. T1E CAR-ODD was cloned into the SFG vector using AgeI and XhoI restriction endonucleases (New England Biolabs) to cleave AgeI and XhoI restriction enzyme sites in the SFG plasmid and those which had been built into the T1E CAR-ODD cDNA. Vector and constructs that had been restriction endonuclease digested were purified using QIAquick PCR purification kit (QIGEN) and ligated using T4 ligase (Thermo Fisher scientific) prior to transformation into One Shot Stb13™ chemically competent E. coli (Thermo Fisher Scientific).

Transformed E. coli were selected using ampicillin (Santa Cruz Biotechnology) containing Luria Bertani (LB) Agar (Sigma-Aldrich) plates. Transformed colonies were there grown up in LB broth (Sigma-Aldrich) with 100 μg/ml ampicillin and then purified using either QIAGEN Plasmid Midi or Maxi kits. Final constructs were sequence verified (Source BioScience). Using a similar approach, the following additional modifications were made: The constitutive reporter construct was generated using a Click Beetle Luciferase (Luc) and eGFP, separated by a viral P2A sequence, reporter construct previously generated in the lab. This was achieved by PCR amplification using Platinum Pfx DNA polymerase (Thermo Fisher Scientific) according to the manufacturer's protocol with the forward primer 5′-CCATGGTGAAGCGTGAGAAAAATG-3′ (SEQ ID NO: 40) and the reverse primer 5′-CTCGAGTTACTTGTACAGCTCGTCCATGC-3′ (SEQ ID NO: 41). The amplified product was digested with NcoI and XhoI (New England Biolabs) and cloned into the SFG vector using the NcoI and XhoI and T4 DNA ligase (Thermo Fisher Scientific). Full length ODD (as described above) was also appended onto the C-terminus of Luc from the reporter construct by overlap PCR using the primers: forward 5′-GAGAAGGCCGGCGGTGCCCCAGCCGCTGGA-3′ (SEQ ID NO: 42) and reverse 5′-CCTCAAAGCACAGTTACAGTATTCCAGGGAAGCGGAGCTACTAACTTCAG-3′ (SEQ ID NO: 43) to amplify the ODD flanked with complimentary overhangs. Subsequently, overlapping fusion PCR using primers: forward 5′-CCATGGTGAAGCGTGAGAAAAATG-3′ (SEQ ID NO: 44) and reverse 5′-CTCGAGTTACTTGTACAGCTCGTCCATGC-3′ (SEQ ID NO: 45) was performed to generate a fragment encoding Luciferase-ODD-P2A-eGFP flanked by NcoI and XhoI restriction sites, which were used to insert Luciferase-ODD-P2A-eGFP into the SFG vector. The HRE modification was targeted in the 3′ LTR of the SFG retroviral vector, as the 3′ LTR region gets copied to the 5′ LTR upon integration. DNA containing 9 tandem 5′-GGCCCTACGTGCTGTCTCACACAGCCTGTCTGAC-3′ (SEQ ID NO: 27) HRE motifs containing both HIF-binding and ancillary site was synthesized as a gBlock® (Integrated DNA Technologies) and sub-cloned into the 3′ LTR of the SFG vector between the NheI and XbaI restriction endonuclease sites using the NheI and Xba1 restriction endonucleases (New England Biolabs). The T1E CAR CD3⁻ truncated control construct was synthesized as a gBlock® (Integrated DNA Technologies) with flanking SbfI and XhoI restriction sites and sub-cloned into the HRE-modified SFG vector using SbfI and XhoI restriction endonucleases (New England Biolabs). To generate the bicistronic Luciferase-T2A-CAR construct, a gBlock® (Integrated DNA Technologies), which was designed to include Luciferase-T2A-T1E peptide binder flanked with AgeI and NotI restriction sites, was inserted into the T1E CAR construct.

Bacterial Transformation

One Shot Stb13 Chemically Competent E. coli (ThermoFisher Scientific) were used for transformations. 5 μl of the ligation mixture was added into a vial of One Shot Stb13 cells that were thawed on ice. Cells were subsequently incubated on ice for 30 minutes. Next, the cells were heat-shocked (45 seconds, 42° C.), placed on ice for 2 minutes then 250 μl of S.O.C. Media was added and the vial incubated in a 37° C. bacterial shaker. The cells were spread on ampicillin (100 μg/ml) agar plates and incubated overnight at 37° C. in a humidified bacterial incubator. Colonies were picked and grown in 3 ml LB broth containing 100 μg/ml ampicillin. DNA was extracted from bacteria using QIAprep Miniprep Kit (Qiagen) according to the manufacturers protocol. DNA was quantified by nanodrop spectrophotometer at 280 nm and sequenced by Source BioScience. SnapGene software was used for sequencing alignments and verification.

Cell Lines

All cell lines were grown at 37° C. and 5% CO₂ in a humidified incubator. Human embryonic kidney (HEK) 293, Phoenix-ECO (gift from Sandra Diebold), human fibrosarcoma cell line HT1080, BW5147.G.1.4 (purchased from ATCC), Jurkat (Clone E6-1) (ATCC) were maintained in RPMI 1640 medium (Gibco) supplemented with 10% foetal calf serum (FCS; Thermo Fisher Scientific). T47D cells were maintained in RPMI 1640 medium (Gibco) supplemented with 10% FCS and insulin (0.2 U/ml).

SKOV3 human ovarian adenocarcinoma cells were originally purchased from ATCC and were re-authenticated for this study by ATCC. HN3 human head and neck adenocarcinoma were acquired from Ludwig Institute for Cancer Research, London and grown in D10 medium, Dulbecco's modified Eagle's medium (DMEM; Gibco) supplemented with 10% FCS and GlutaMAX (Thermo Fisher Scientific). Murine Lewis Lung carcinoma (LL2) cells were purchased from ATCC and were cultured in RPMI 1640 supplemented with 10% FCS. Cell lines were confirmed to be free of Mycoplasma for this study using the MycoAlert® Mycoplasma Detection Kit (Lonza).

Mice

NSG (NOD-scid IL2Rgamma^{null}) mice were purchased from Charles River and bred internally. Balb/c Rag2^{-/-} mice were a gift from Professor Adrian Hayday (KCL). Male mice were used for studies involving HN3 and female mice were used for studies involving SKOV3 and LL2 studies. All mice used for ectopic tumor studies were 6-8 weeks old and approximately 22 g in weight.

Generation of Retrovirus

To produce retrovirus with tropism for human cells, RD114 pseudotyped transient retroviral particles were generated by triple transfection using (per well of a six well plate) 1.5 μg of Peq-Pam plasmid (Moloney GagPol), 1 μg RDF plasmid (RD114 envelope) and 1.5 μg of the SFG plasmids using FuGENE HD transfection reagent into 50%-60% confluent HEK 293T cells (Promega, US). Peq-Pam, RDF and SFG plasmids were incubated in plain RPMI 1640 media (Gibco) for 15 minutes at room temperature (RT) and then added drop-wise onto the 293T cells. Retrovirus-containing supernatant was harvested after 48 hours and used to transduce human cell lines.

Hypoxic Conditions

A hypoxia chamber was purchased from STEMCELL Technologies (Canada) and purged with certified gas supplied by BOC containing 0.1%, 1% or 5% O₂, with constant 5% CO₂ and using N2 as a balance. The chamber was re-purged 1 hour after the first purge according to the manufacturer's protocol. Equal numbers of cells plated on two parallel plates where one was exposed to hypoxic conditions and the other maintained at normoxia for 18 hours. Luciferase activity was then measured using a luciferase assay (Promega, US) according to the manufacturer's protocol on a Perkin Elmer Fusion α-FP plate reader (Life Sciences). Incubation time for assessing hypoxia responsive gene expression was based on known studies. Hypoxic conditions were also mimicked using cobalt (II) chloride (Sigma-Aldrich, US) (PHD inhibitor) at a final concentration of 100 μM.

Western Blot Analysis

Cells were lysed in Western lysis buffer (2.5 ml 1M Tris pH 6.8, 1 g SDS, 5 ml glycerol, 17.5 ml water) containing a 1× concentration of a protease inhibitor cocktail (Thermo Scientific). Total protein in cell lysate was quantified using Pierce BCA Protein Assay Kit (ThermoFisher Scientific). 10 ug of protein from each lysate alongside with SeeBlue pre-stained protein ladder (ThermoFisher Scientific) were separated using 12% sodium dodecyl sulphate polyacrylamide gel electrophoresis (SDS PAGE) at 150V and transferred onto an activated PVDF nitrocellulose membrane (Thermo Scientific, Pierce) at 30V for 2 hours. The membrane was blocked with 1% milk in PBS 0.1% Tween-20 for 1 h at RT and then incubated with rabbit anti-HIF1α antibody (Novus Biologicals, Littleton, Colo.) in 1% milk (1:2000) overnight at 4° C. or polyclonal anti-β-Actin (1:5000; Abcam). After washing, the membrane was incubated with a secondary anti-rabbit horseradish peroxidase (HRP) goat anti-rabbit IgG antibody in 1% milk (1:5000; Invitrogen). Next, the HRP substrate 3,3′,5,5′ tetramethylbenzidine (TMB) was added to the PVDF membrane and the signal was read using a CL-XPosure Film (Thermo Scientific) and Western blot X-ray analyser.

Quantitative PCR

Genomic DNA was extracted from cell lines using a DNeasy Blood & Tissue Kit (QIAGEN, Germany) according to manufacturer's protocol and measured with nanodrop spectrophotometer at 280 nm absorbance. qPCR was performed using KiCqStart SYBR Green qPCR ReadyMix with ROX (purchased from Sigma-Aldrich, US) according to the manufacturer's protocol using custom designed primers to generate amplicons from Tbp, Luc or T2A sequences in the genome. The primers used were: murine Tbp 5′-TGTCTGTCGCAGTAAGAATGGA-3′ (SEQ ID NO: 46) and 5′-AAAATCCCAGACACGGTGGG-3′ (SEQ ID NO: 47), human Tbp 5′-TTTGGTGTTTGCTTCAGTCAG-3′ (SEQ ID NO: 48) and 5′-ATACCTAGAAAACAGGAGTTGCTCA-3′ (SEQ ID NO: 49), Luc 5′-ATTTGACTGCCGGCGAAATG-3′ (SEQ ID NO: 50) and 5′-AAGATTCATCGCCGACCACAT-3′ (SEQ ID NO: 51), T2A 5′-CGGAGAAAGCGCAGC-3′ (SEQ ID NO: 52) and 5′-GGGTCCGGGGTTCTCTT-3′ (SEQ ID NO: 53). Amplifications of the genes of interest were detected on an ABI 7900HT Fast Real Time PCR instrument (ThermoFisher Scientific).

Quantitative Reverse Transcription PCR

Healthy female C57BL/6 mice were sacrificed and the following organs were extracted: mammary gland, fat, liver, kidneys, colon, small intestine, stomach, skeletal muscle, lung, heart, brain, olfactory bulb and eyes (n=13). Organs were submerged in RNAlater (Sigma-Aldrich, US) reagent to stabilise and protect cellular RNA and kept overnight at 4° C. RNA was isolated from the tissues using PrepEase RNA Spin Kit (Affymetrix, US) according to the manufacturer's protocol and quantified using NanoDrop spectrophotometer at 280 nm. Erbb1-4 and Integrin β-6 mRNA expression was analyzed in purified mRNA by quantitative reverse transcriptase PCR using the EXPRESS One-step Superscript qRT-PCR kit (ThermoFisher Scientific), alongside assays on demand for the genes of interest which included: Egfr Mm01187858_m1, Erbb2 Mm00658541_m1 Erbb3 Mm01159999_m1, Erbb4 Mm01256793_m1, Itgb6 Mm01269869_m1, Tbp Mm01277042_m1. qRT PCR was performed using an ABI 7900HT Fast Real Time PCR instrument (ThermoFisher Scientific) and data analysis was done in Excel. RNA was stored at −80° C. Expression of all genes is represented relative to the house-keeping gene Tata-binding protein (Tbp).

List of Primers Used:

| Primer name | Sequence |
|---|---|
| Fwd EPO HRE | 5′-CCA CCT GTA GGT |
| | TTG GCA AGC TAG CGT |
| | CCG GGA AAC-3′ |
| | (SEQ ID NO: 54) |
| Fwd GLUT3 HRE | 5′-CCA CCT GTA GGT |
| | TTG GCA AGC TAG CCA |
| | CGC CTG TAA TC-3′ |
| | (SEQ ID NO: 55) |
| fwd VEGFA HRE | 5′-CCA CCT GTA GGT |
| | TTG GCA AGC TAG CCC |
| | CCC TTT GGG-3′ |
| | (SEQ ID NO: 56) |
| Fwd frag 3 | 5′-GAA CCA TCA GAT |
| downstream | GTT TCC AGG-3′ |
| Xba HRE | (SEQ ID NO: 57) |
| Fwd frag A | 5′-ATC CGC CAC AAC |
| binds in eGFP | ATC GAG-3’ |
| | (SEQ ID NO: 58) |
| Rev EPO HRE | 5′-CCT GGA AAC ATC |
| | TGA TGG TTC TCT AGA |
| | CCT CAG GCC CGG-3′ |
| | (SEQ ID NO: 59) |
| Rev frag 3 | 5′-GCG GGC CTC TTC |
| downstream | GCT ATT A-3′ |
| EcoRI | (SEQ ID NO: 60) |
| Rev frag A | 5′-TTG CCA AAC CTA |
| upstream Nhe | CAG GTG G-3′ |
| HRE | (SEQ ID NO: 61) |
| fwd HRE from | 5’-GGT GGT ACC GGT |
| p3 p4 p5 | CTG TAG GTT TGG CAA |
| | GCT AGC-3′ |
| | (SEQ ID NO: 62) |
| fwd primer | 5′-GAA AGA CCC CAC |
| seq genome to | CTG TAG GTT T-3′ |
| verify | (SEQ ID NO: 63) |
| orientation | |
| of HRE | |
| fwd puro plus | 5′-GCC ACG ACC GGT |
| AgeI plus | GCC GCC ACC ATC CCC |
| buffering | TGA CCC ACG CC-3′ |
| | (SEQ ID NO: 64) |
| fwd tataa | 5′-GGG TAT ATA ATG |
| linker gilbert | GAA GCT CGA ATT CTA |
| overlap | GCG-3′ |
| | (SEQ ID NO: 65) |
| fwr HRE overlap | 5′-CGA AAG GAG CGC |
| and skip | ACG ACC AAT TCA ATT |
| Nco | GGC CCT ACG TG-3′ |
| | (SEQ ID NO: 66) |
| gagSFG seq primer | 5′-CGG ATG GCC GCG |
| | AGA-3′ |
| | (SEQ ID NO: 67) |
| qPCRfwd Luc | 5′-ATT TGA CTG CCG |
| | GCG AAA TG-3′ |
| | (SEQ ID NO: 68) |
| qPCRfwdrefmouseTBP | 5’-TGT CTG TCG CAG |
| | TAA GAA TGG A-3′ |
| | (SEQ ID NO: 46) |
| qPCRreffwdhumanTBP | 5′-TTT GGT GTT TGC |
| | TTC AGT CAG-3′ |
| | (SEQ ID NO: 48) |
| qPCRrefrevhumanTBP | 5′-ATA CCT AGA AAA |
| | CAG GAG TTG CTC |
| | A-3′ |
| | (SEQ ID NO: 49) |
| qPCRrefrevmouseTBP | 5′-AAA ATC CCA GAC |
| | ACG GTG GG-3’ |
| | (SEQ ID NO: 47) |
| qPCRrev Luc | 5′-AAG ATT CAT CGC |
| | CGA CCA CAT-3′ |
| | (SEQ ID NO: 69) |
| rev GLUT3 HRE | 5′-CCT GGA AAC ATC |
| | TGA TGG TTC TCT AGA |
| | TTT GGC CAT GTT |
| | GAC TAG-3′ |
| | (SEQ ID NO: 70) |
| rev VEGFA HRE | 5′-CCT GGA AAC ATC |
| | TGA TGG TTC TCT AGA |
| | GTT CCG GGG TTA GTC |
| | AGT-3′ |
| | (SEQ ID NO: 71) |
| rev primer seq | 5′-CAC CAA AGA GTC |
| orientation | CTA AAC GAT C-3′ |
| HRE | (SEQ ID NO: 72) |
| rev puro skip | 5′-CAC GTA GGG CCA |
| Nco site | ATT GAA TTG GTC GTG |
| | CGC TCC TTT CG-3′ |
| | (SEQ ID NO: 73) |

Cell Viability

Cells were washed twice with cold Dulbecco's Phosphate Buffered Saline (DPBS) (Gibco) and resuspended in 1× Binding Buffer supplied in the PE Annexin V Apoptosis Detection Kit (BD Biosciences). Cells were then stained with PE Annexin V and 7-Amino-Actinomycin (7-AAD) according to PE Annexin V Apoptosis Detection Kit protocol (BD Biosciences) for 15 minutes at RT in the dark, washed and resuspended in 1× Binding Buffer and analysed by flow cytometry (FACSCanto II Flow cytometer, BD Biosciences). Flow data were analysed using FlowJo software. PE Annexin V and 7-AAD negative cells are considered viable, PE Annexin V positive and 7-AAD negative cells are in early apoptosis and PE Annexin V and 7-AAD positive cells are in late apoptosis or dead.

T-Cell Isolation

For isolating human T-cells; blood was obtained from healthy volunteers under approval of the Guy's and St Thomas' Research Ethics Committee (REC reference 09/H0804/92). Blood was collected into Falcon tubes containing anti-coagulant (10% Citrate), mixed at 1:1 with RPMI 1640 and layered over Ficoll-Paque Plus (GE Healthcare). Samples were centrifuged at 750 g for 30 mins at 20° C. to separate the peripheral blood mononuclear (PBMC) cell fraction. The interface between the plasma and the Ficoll layer, which contained the PBMCs, was harvested using a sterile Pasteur pipette and washed in RPMI 1640. T-cells were purified from the PBMC fraction using human Pan T-cell isolation kit (Miltenyi Biotec) and isolated using a MidiMACs™ separator and LS columns (Miltenyi Biotec) according to the manufacturer's protocol. Purified human T-cells were activated using CD3/CD28 Human T-Activator Dynabeads (Gibco) at a 1:1 cell to bead ratio and seeded in tissue culture plates at 3×10⁶ in RPMI 1640 supplemented with 5% human serum (Sigma-Aldrich) and 1× penicillin/streptomycin. The following day, 100 IU/ml recombinant human IL-2 (PROLEUKIN) was added to the cultures.

T-Cell and Cell Line Transduction

To produce retrovirus with tropism for human cells, RD114 pseudotyped retroviral particles were generated by triple transfection, using Peq-Pam plasmid (Moloney GagPol), RDF plasmid (RD114 envelope) and the SFG plasmid of interest, using FuGENE HD transfection reagent (Promega), of HEK 293T cells as previously described. To produce retrovirus with murine cell tropism, Phoenix-ECO retrovirus producer cells were transfected using FuGENE HD (Promega) with the relevant plasmid. Supernatant containing viral particles were harvested and incubated with the cells of interest for at least 48 h to allow their transduction. T-cells were transduced in non-tissue culture treated plates that were pre-coated with 4 μg/cm² RetroNectin (Takara Bio) overnight at 4° C. Prior to the retroviral transduction of human T-cells, CD3/CD28 Human T-Activator Dynabeads (Gibco) were removed and fresh IL-2 was added as stated in the T-cell isolation section. In the case of T-cell transduction with the bicistronic 4αβ-T2A-CAR construct, following T-cell transduction, human IL-4 (Peprotech) at 30 ng/ml final concentration was added to the culture to enrich the transduced T-cell population. Adherent cell lines, including SKOV-3 and HN3, were transduced with retrovirus, produced as indicated before, in media solution containing Polybrene (Santa Cruz Biotechnology Inc) at 4 μg/ml final concentration to increase infection efficiency. Cells modified to express Luc/eGFP were purified by cell sorting using BD FACSAria III (BD Biosciences) based on their eGFP fluorescence.

In Vitro Studies

In vitro hypoxia was achieved using a hypoxia incubator chamber (Stemcell Technologies) purged at 25 L/min for 4 mins with gas containing either; 0.1, 1, 5% O₂, 5% CO₂ and nitrogen as a balance (BOC), after which the chamber was sealed. This process was repeated again after 1 h. Hypoxia-mediated HIF1α stabilization was, in some cases, mimicked by using the chemical CoCl₂ (Sigma-Aldrich), which inhibits HIF1α hydroxylation, at 100 μM final concentration, unless otherwise stated. In vitro cytotoxicity assays 1×10⁴ Luc/eGFP-expressing SKOV3 cells were seeded in 96-well tissue culture plates and transduced or non-transduced T-cells were added in the well at the indicated effector to target ratios. Co-cultures were incubated for 24, 48 and 72 h time points and target cell viability was determined by luciferase quantification (in normoxic conditions, following the addition of 1 μl of 15 mg/ml XenoLight D-luciferin (PerkinElmer) in PBS per 100 μl of media. Luminescence was quantified using a FLUOstar Omega plate reader (BMG Labtech). At the 24 and 48 h co-culture time points a sample of media was taken from the co-culture and subsequently used for IL-2 and IFNγ quantification, respectively. IL-2 was quantified using Human IL-2 ELISA Ready-SET-Go! Kit, 2nd Generation (eBioscience) as per manufacturer's protocol. IFNγ was quantified using Human IFN-gamma DuoSet ELISA kit (Bio-Techne) as per manufacturer's protocol. In both ELISAs cytokine concentration was determined by absorbance measurements at 450 nm on a Fusion alpha-FP spectrophotometer (Perkin-Elmer).

In Vivo Studies

Tumour cell lines (2.5×10⁵ cells in PBS) were inoculated by subcutaneous (s.c.) injection into female (for SKOV3 and LL2) and male (for HN3) mice that were six to eight weeks of age. Once tumours were palpable, digital caliper measurements of the long (L) and short (S) dimensions of the tumour were performed every 2 or 3 days. Tumour volume was established using the following equation: Volume=(S²×L)/2. Blood samples were taken from mice in EDTA-coated Microvette™ tubes (Sarstedt) and plasma was extracted by centrifugation of these samples at 2,000 g for 5 mins. The indicated doses of CAR T-cells were injected in 200 μl PBS through the tail vein using a 30 G needle. Tumour tissue, and other organs, for flow cytometry analyses were enzyme-digested to release single cells as previously described. In brief, tissues were minced using scalpels, and then single cells were liberated by incubation for 60 mins at 37° C. with 1 mg/ml Collagenase 1, from Clostridium Histolyticum (Sigma-Aldrich) and 0.1 mg/ml Deoxyribonuclese I (AppliChem) in RPMI (Gibco). Released cells were then passed through a 70 μm cell strainer prior to staining for flow cytometry analyses. Viable cells were numerated using a haemocytometer with trypan blue (Sigma-Aldrich) exclusion.

Bioluminescence Imaging

To assess luciferase bio-distribution in vivo, mice were injected intraperitoneally (i.p.) with 200 μl (15 mg/ml) XenoLight D-luciferin (PerkinElmer) in sterile PBS 10 mins prior to imaging. Animals were anesthetized for imaging and emitted light was detected using the In vivo Imaging System (IVIS®) Lumina Series III (PerkinElmer) and data analysed using the Living Image software (Perkin Elmer). Light was quantified in photons/second/unit area.

Flow Cytometry

Flow cytometry was performed as previously described. The following antibodies were purchased from eBioscience and were used at 1 μg/ml unless stated otherwise: anti-human CD3ε Brilliant Violet 421™ (SK7; Biolegend®), anti-human CD8α Alexa Fluor 488 (RPA-T8), anti-human CD4 PE (RPA-T4), anti-human CD45 Brilliant Violet 510™ (H130 Biolegend®), anti-mouse CD4 FITC (Clone: RM4-5), anti-mouse CD8α eFluor®450 (Clone: 53-6.7), anti-mouse CD3ε PE (Clone: 145-2C11), neutralizing anti-mouse CD16/CD32 (Clone: 2.4G2). Background staining was established using fluorescence minus one stained samples. T1E CAR was stained with a biotinylated anti-human EGF antibody (Bio-Techne: BAF236) and detected using Streptavidin APC. eGFP was detected by its native fluorescence. Dead cells and red blood cells were excluded using 1 μg/ml 7-amino actinomycin D (Cayman Chemical Company) alongside anti-Ter-119 PerCP-Cy5.5 (Ter-119; eBioscience). Data were collected on a BD FACS Canto II (BD Biosciences). Data was analyzed using FlowJo software (Freestar Inc.).

Statistics

Normality and homogeneity of variance were determined using a Shapiro-Wilk normality test and an F-test respectively. Statistical significance was then determined using a two-sided unpaired Students t test for parametric or Mann-Whitney test for nonparametric data using GraphPad Prism 6 software. When comparing paired data, a paired ratio Students t test was performed. A Welch's correction was applied when comparing groups with unequal variances. Statistical analysis of tumour growth curves was performed using the “CompareGrowthCurves” function of the statmod software package. No outliers were excluded from any data presented.

Results

HRE Design

Based on analysis of genomic data obtained from the Ensembl database, putative HIF1-binding site (HBS), which is conserved between species and between hypoxia-induced genes, were identified. We compared the putative 6 nucleotide (nt)-long HBS from different oxygen-sensitive genes in human, mouse and rat based on the frequency of each nucleotide in each position in the 6-nt sequence, which binds HIF, and a sequence logo was constructed for human and mouse HBS (FIGS. 4A and 4B). Outside of the HBS element there is also a sequence 8 nts downstream of the genomic HBS sequence, which is associated with oxygen-controlled transcription. This site is known as HIF ancillary site (HAS) (FIG. 4C).

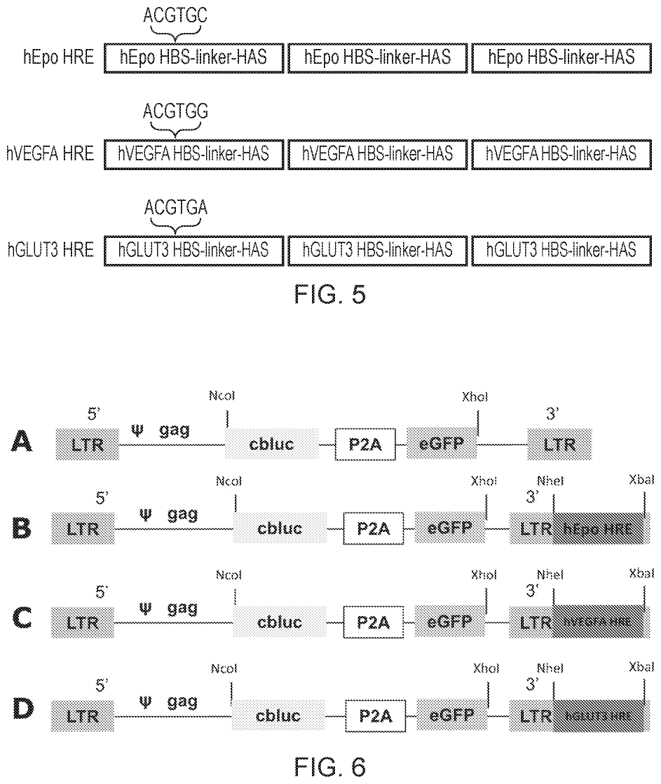

The HRE design included an HBS and a HAS site separate by a 8 nt linker region taken from the genomic sequence. In the first instance, 3 sequential HBS-HAS sequences were used. Also, to see whether different HBS sequences have different sensitivities to HIF, three constructs were initially designed, each containing 3 sequential HBS-HAS (HRE for simplicity) sequences. The difference between these constructs was that the HBS in each construct was derived from different genes (FIG. 5). These genes were human Epo, human VEGFA and human GLUT-3.

HREs in the LTR

To stably integrate the construct into the host cell's genome we used the SFG retroviral vector with modified LTRs as previously described. The SFG vector is derived from the Moloney murine leukaemia virus (MMLV). We attempted to modify the retroviral enhancer region within the LTRs without affecting the integration of the transgene into the host cell genome. This has previously been achieved by cloning HREs in to the NheI/XbaI site of the LTR, which is upstream the viral promoter. In order to avoid inactivating the vector or its ability to integrate into the host genome, we replaced the NheI/XbaI region with a fragment of similar length.

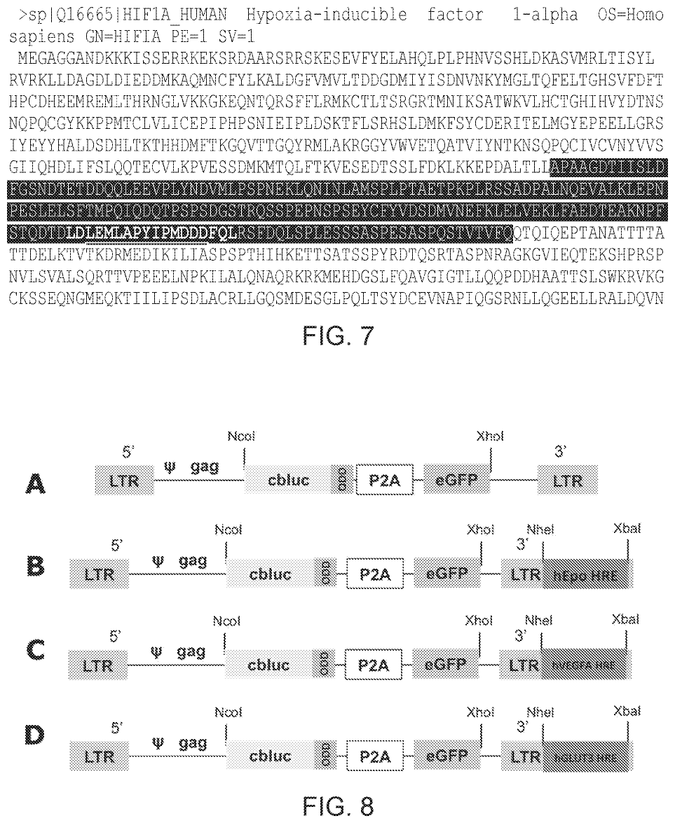

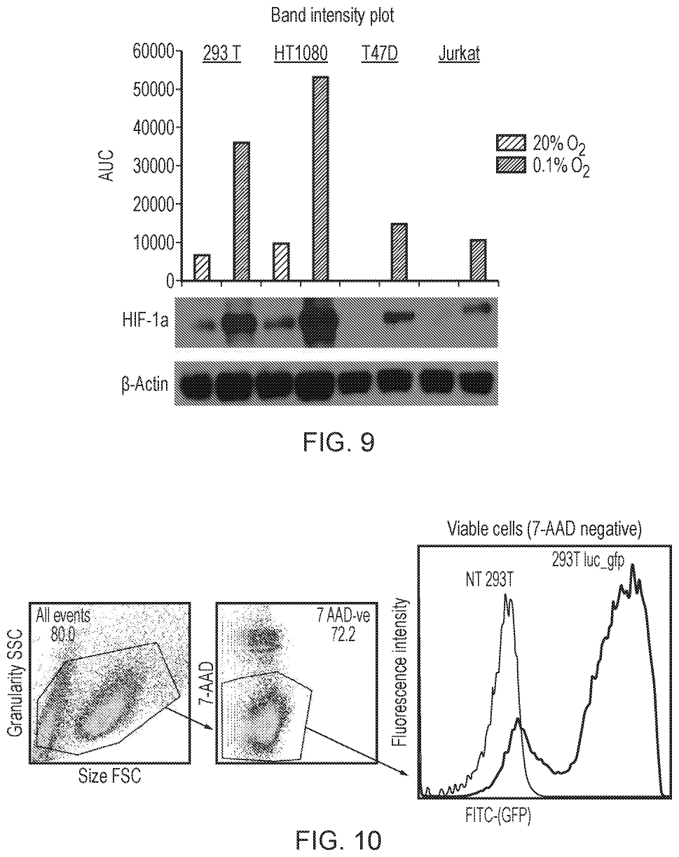

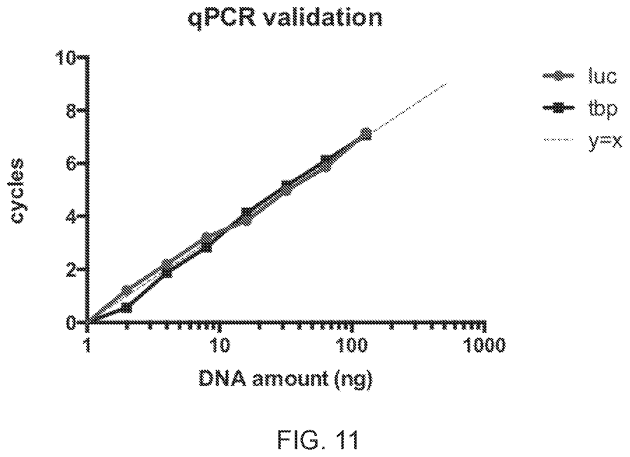

DNA sequences containing our HREs sequences that include 5′ NheI and 3′ XbaI restriction sites were synthesized by GeneArt. These sequences were sub-cloned in the NheI/XbaI site in the 3′ LTR of the SFG MMLV vector. We modified the 3′ LTR but not the 5′ LTR as, when reverse transcription occurs, the modified 3′ LTR U3 region is copied to the 5′ LTR. Due to the fact that NheI/XbaI were not unique restriction sites in the SFG, we synthesised a fragment in several steps using sequential overlapping PCR, which contained unique restriction sites (XhoI/EcoRI) in order to achieve specific modification of the NheI/XbaI site in the 3′ LTR. To make an oxygen-sensing reporter construct, green-emitting variant of click beetle luciferase and green fluorescent protein separated by a P2A peptide (self-cleaving peptide) were cloned into NcoI/XhoI site of the SFG vector. The resulting constructs are shown in FIG. 7.

ODD Addition

We simultaneously cloned an additional set of vectors that had an ODD domain attached to the luciferase reporter to facilitate protein degradation under conditions of normoxia. HIF1α stability is controlled by oxygen-dependent hydroxylation of prolines (p402 and p564) in the ODD. This sequence was fused with a protein of interest to make the degradation of the protein oxygen-dependent. Based on the UniProt database, the ODD domain (highlighted in FIG. 7) of human HIF1α is 203 amino acids long while the mouse orthologue consists of 213 amino acids. Using overlapping PCR, we fused the amino acid sequence 557-574 from HIF1α (in bold in FIG. 7) to the C-terminus of luciferase. The exact amino acid sequence 557-574 (LDLEMLAPYIPMDDDFQL (SEQ ID NO: 74)) is conserved in human and mouse. The resulting fragment (luciferase-ODD fusion) was inserted into the LTR-modified and LTR-unmodified SFG reporter constructs, as depicted in FIG. 8.

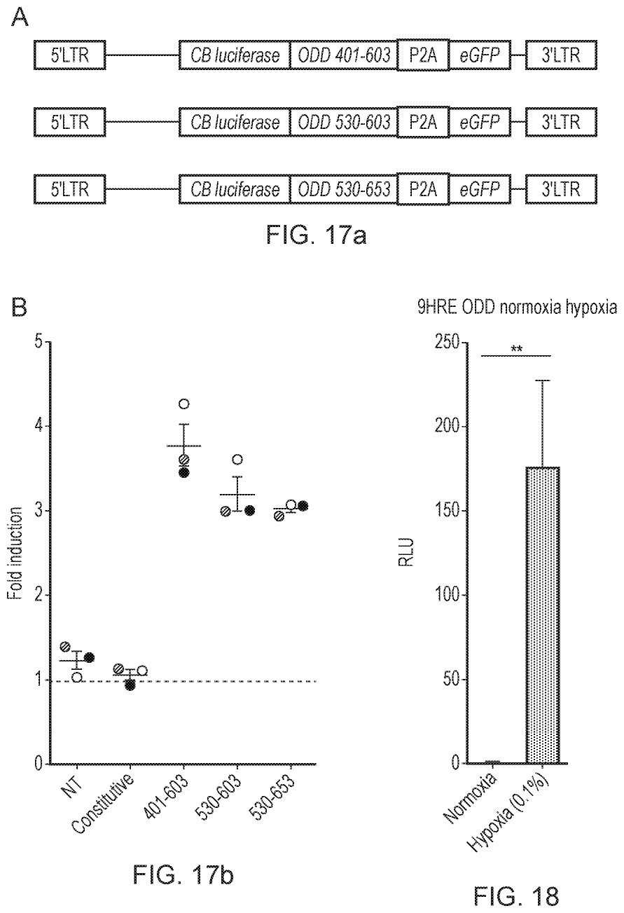

In subsequent experiments we fused SEQ ID Nos 29, 30, 31. All three SEQ ID Nos conferred oxygen sensitivity to the fusion partner, with optimal results being obtained with SEQ ID NO: 29, i.e. whole ODD (401-603) (FIG. 17).

HIF1α Stability Under Normoxia or Hypoxia in Different Cell Lines

Cell lines were cultured for 18 hours in normoxic or hypoxic conditions, 20% or 0.1% O₂, respectively. The following human cell lines were screened under these conditions: HEK293 T, HT1080, T47D and Jurkat (Clone E6-1). Immediately after the 18-hour exposure, cells were lysed and a Western blot was performed to quantify HIF1α as described in the methods. In all cell lines tested, HIF1α was found to be stabilised under hypoxic conditions (0.1% O₂), when compared to normoxia (20% O₂) (FIG. 10). Protein was quantified using densitometry using ImageJ Software. 293T cells and HT1080 cells had the highest amount of HIF1α under hypoxic conditions, however in these cell lines there was also some HIF1α detected in normoxic conditions. T47D and Jurkat cells both had detectable HIF1α protein under hypoxic conditions but no detectable HIF1α band was seen for T47D and Jurkat cells under normoxic conditions.

Cell Choice

We chose to use 293T cells in initial experiments for three reasons. First, HIF1α Western blot analysis showed that 293T cells had strong expression of HIF1α protein under hypoxic conditions, at levels 5-fold higher than found in normoxia. Second, we observed that 293T are fast-growing cells when compared to T47D, allowing multiple experiments to be performed in a short time period. Third, 293T cells are the packaging cell lines that we use to produce the retrovirus. Therefore, transfection of 293T cells to produce retrovirus results in an auto-transduction of the 293T cells themselves.

Transduction Efficiency Based on Flow Cytometry

Since the expression of transgene in our constructs is oxygen-sensitive, we cannot rely on flow cytometry to determine accurate transduction efficiency. Flow cytometry analysis of 293T cells, which had been transduced with the constitutive luciferase-P2A-GFP construct (SFG Reporter construct), revealed a transduction efficiency in the live cell population (7-AAD negative) of 83% (FIG. 10). These results indicated that retroviral transduction method we used worked efficiently.

Sequencing to Verify Post-Integration HRE Orientation within the LTR

To confirm that the modifications in the 3′ LTR had been duplicated to the 5′ LTR and were correctly orientated in the integrated provirus we sequenced the 5′ LTR region after transduction. Genomic DNA was isolated from transduced 293T cells and the 5′ LTR region was amplified via PCR and run on a 1.2% agarose gel. The band of the correct length was excised, gel purified and then sequenced. Sequence analysis revealed that the HRE modifications to the 3′ LTR were correctly copied and had the correct orientation in the 5′ LTR.

Establishment of Copy Number Assay/qPCR (Copy Number) Assay Validation

For our assay in which we would quantitate luciferase expression under hypoxic conditions, we need to normalise our data, as not every cell would be transduced and some cells may have contained multiple copies of the reporter construct. To permit this we utilised quantitative PCR (qPCR) using the amplification of a reference gene (TBP), which is present as 2 copies in every cell (native genomic DNA), as well as that of the transgene (luciferase) to allow us to calculate the number of integrated transgenes. To design the qPCR primers, we screened multiple possible primer sequences in silico using the Ensembl database to ensure high specificity of binding. We chose primers that bind to unique sites in the genes of interest so that the amplicons produced by PCR would be indicative of reference and transgene gene amount. We designed a primer set that binds to click beetle luciferase and human and mouse TBP (since we are using both human and mouse cell lines). Using this approach, the following three sets of primers were designed: forward mouse TBP (5′-TGT CTG TCG CAG TAA GAA TGG A-3′ (SEQ ID NO: 46)) and reverse mouse TBP (5′-AAA ATC CCA GAC ACG GTG GG-3′ (SEQ ID NO: 47)) that amplify a 94 nt fragment specifically from the mouse TBP gene, forward human TBP (5′-TTT GGT GTT TGC TTC AGT CAG-3′ (SEQ ID NO: 48)) and reverse human TBP (5′-ATA CCT AGA AAA CAG GAG TTG CTC A-3′ (SEQ ID NO: 49)) that amplify a 103 nt fragment specifically from the human TBP, and forward luciferase (5′-ATT TGA CTG CCG GCG AAA TG-3′ (SEQ ID NO: 68)) and reverse luciferase (5′-AAG ATT CAT CGC CGA CCA CAT-3′ (SEQ ID NO: 69)), which amplify specifically a 90 nt fragment from luciferase transgene.

To determine primer binding specificity (a single amplified product), we performed qPCR on genomic DNA extracted from cells and run the PCR product on an agarose gel. All PCR products gave a single band of appropriate length demonstrating that the primers were specific.

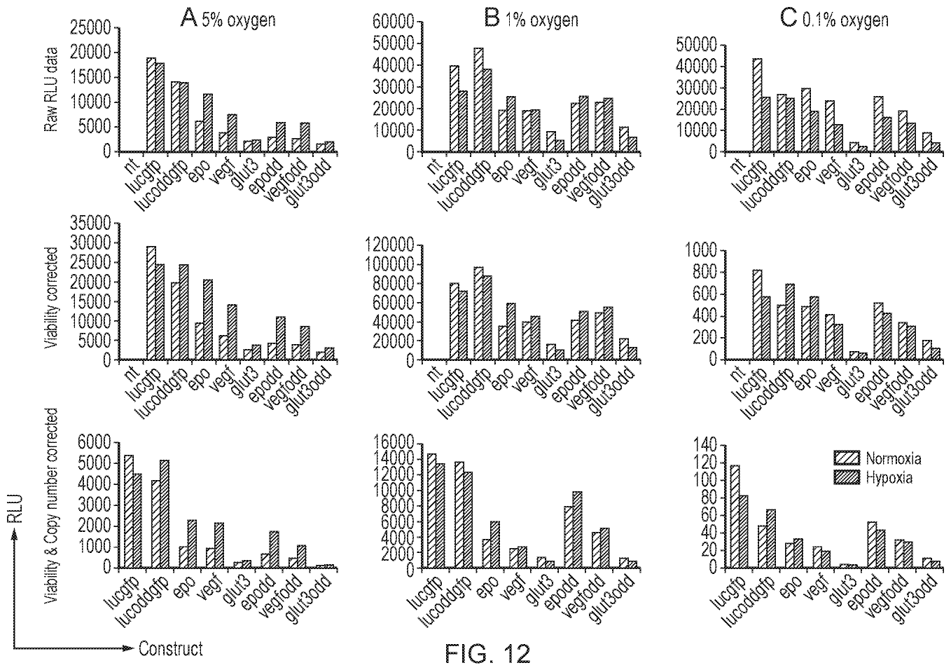

To validate the copy number assay, genomic DNA was extracted from non-transduced cells and from cells transduced with the construct containing the click beetle luciferase. 200 ng of DNA was serially diluted (1:2) and qPCR was performed using the designed primers. Each reaction was performed in triplicate. As expected, no luciferase amplicon was detected in the DNA extracted from non-transduced cells. qPCR data generated using DNA extracted from the transduced cells demonstrated that there was a linear relationship between the qPCR signal from both luciferase and TBP primer sets and the cycle number of the reaction, validating the assay. 18-hour incubation of 293T cells in 20%, 5%, 1% and 0.1% oxygen 293T cells were transduced with retrovirus and transduction efficiency was determined by qPCR. Non-transduced 293T cells and 293T cells transduced with luciferase constructs 1-8 (A, B, C and D from FIGS. 6 and 8) were seeded and cultured in 5%, 1% and 0.1% oxygen and normoxia (20% oxygen). Following an 18-hour incubation under these conditions, luciferase expression, and cell viability were determined. Raw relative light unit (RLU) data obtained following 18 h incubation of 293T cells in 5% oxygen and normoxia indicate that an oxygen-controlled luciferase expression system had been generated (FIG. 14A). All HRE and/or ODD modified constructs gave a modest increase in RLU in hypoxia (5%) compared to normoxia, however this was not seen at lower oxygen concentrations. In general, LTR HRE modified constructs gave lower RLU compared to their LTR wild type counterparts when cells were maintained at 0.1% oxygen. Based on previous publications, more severe hypoxia tends to increase the fold induction in protein expression under the hypoxic vs the normoxic condition. However, we did not see this trend in our data (FIG. 12C).

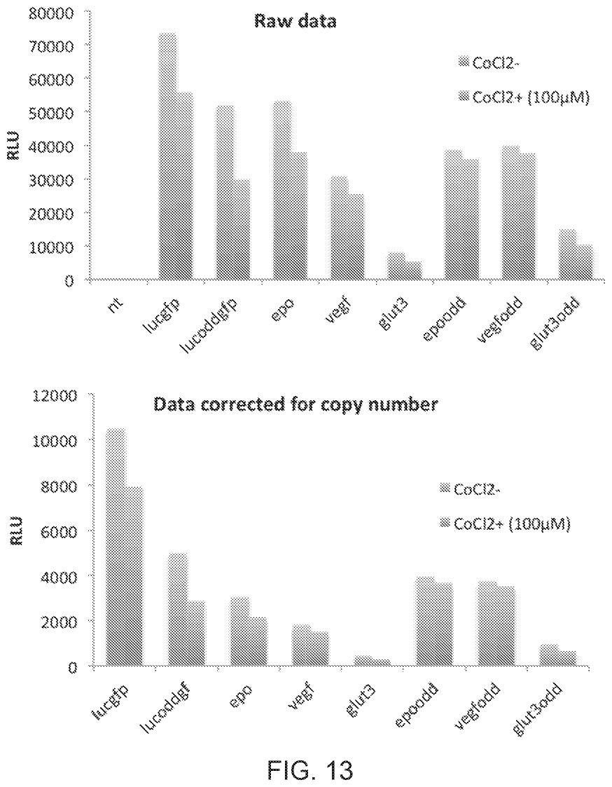

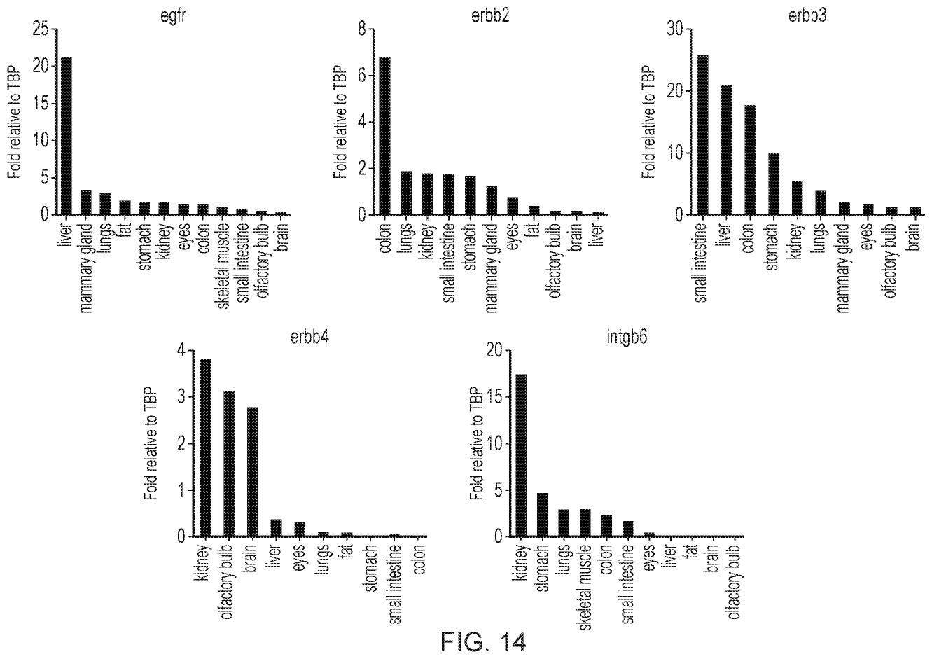

The effect of adding the ODD domain within the construct is best assessed by comparing the constitutively expressing unmodified LTR construct +/−ODD. See FIGS. 17 and 18. The addition of the ODD, across the experiments only modestly decreased the detection of luciferase in the conditions. It remained a possibility that the absence of a significant induction of hypoxia might have been a result of the apparatus or experimental procedure, so to exclude this, we stimulated the transduced 293T cells with 100 μM Cobalt chloride for 18 hours which mimics hypoxic conditions (by cobalt-mediated inhibition of HIF1α degradation). However, we did not observe luciferase induction in the presence of 100 μM Cobalt chloride compared to the absence of Cobalt chloride (FIG. 13).

FIG. 29 demonstrates the superiority of the HRE promoter versus the wild type. We observed that HRE modification leads to a superior promoter, which in a hypoxic, e.g. tumour environment, drives better expression of the downstream gene in comparison with its non-modified wild type counterpart in the same conditions. FIGS. 29 A and B demonstrate that HRE-modification alone leads to superior target killing and activation capacity in T-cells in a hypoxic (solid tumour) environment at all effector:target ratios (even at low E:T such as 1:2). This is extremely important as usually the effector to target ratio in an established solid tumour in patients is low, thus the ability of HRE-CAR to be efficient at low E:T ratios is crucial and may determine CAR T-cell immunotherapy outcome. In addition, this enhanced CAR expression will only happen within the solid tumour because of its hypoxic status and therefore as the enhanced expression will be tumour-specific it would not pose any risk of off tumour toxicities higher than the risk from the WT CAR.

Hypoxia Inducibility in the Presence of Increasing Numbers of HRE Elements in the Promoter

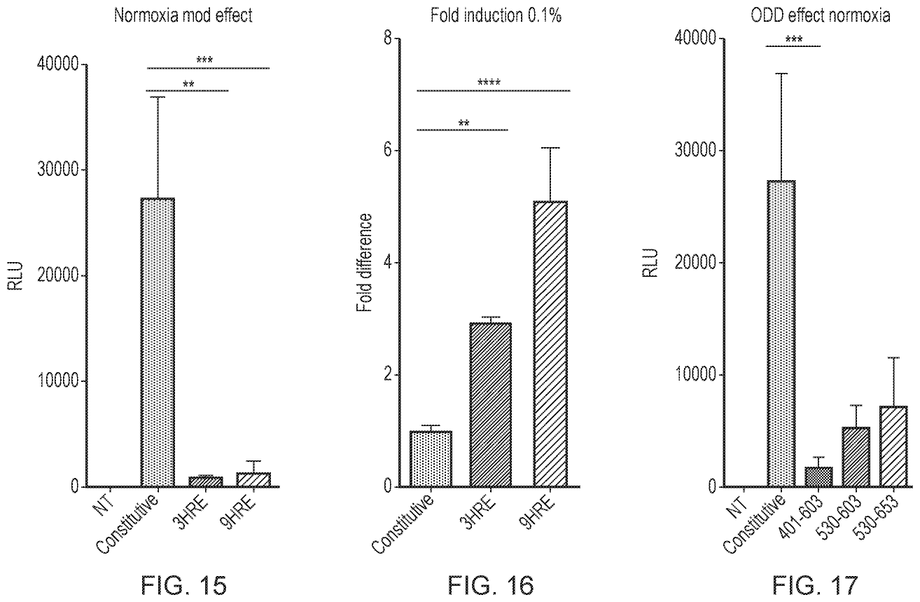

As shown in FIGS. 15 and 16, hypoxia-inducibility increases with increasing numbers of HRE elements in the promoter. By modifying the LTRs (retroviral promoter) to contain multiple HREs, expression of luciferase in conditions of normoxia was effectively silenced.

Luciferase Stability in Normoxia (+/−ODD)

A variety of ODD segments were fused to the C-terminus of luciferase and the results are shown in FIGS. 20 and 21. SEQ ID NO: 29: ODD segment 401-603, SEQ ID NO: 30: ODD segment 530-603 and SEQ ID NO: 31: ODD segment 530-653 were tested. Addition of each of the three ODD segments resulted in reduced expression in normoxic conditions, with the combination of the 9 HRE promoter architecture with SEQ ID NO: 29 (the 401-603 ODD) showing no expression of luciferase in normoxia, but which was switched on in hypoxia (FIG. 18).

In Vitro and In Vivo T4-CAR Results

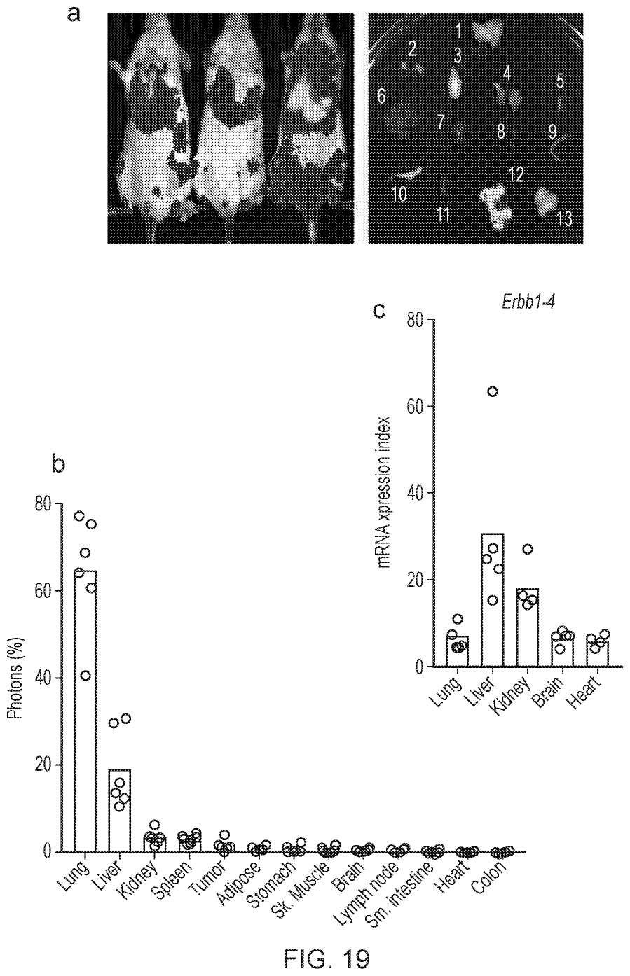

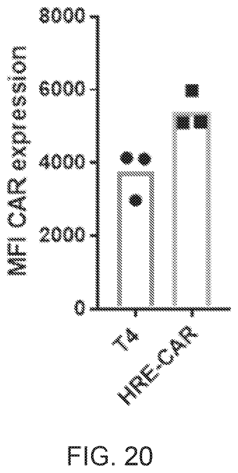

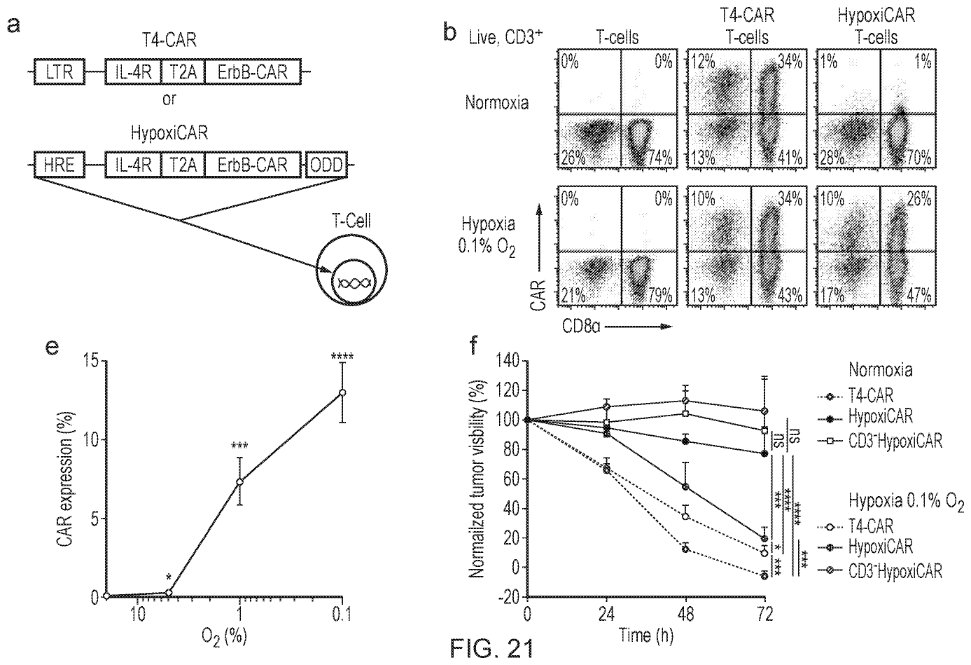

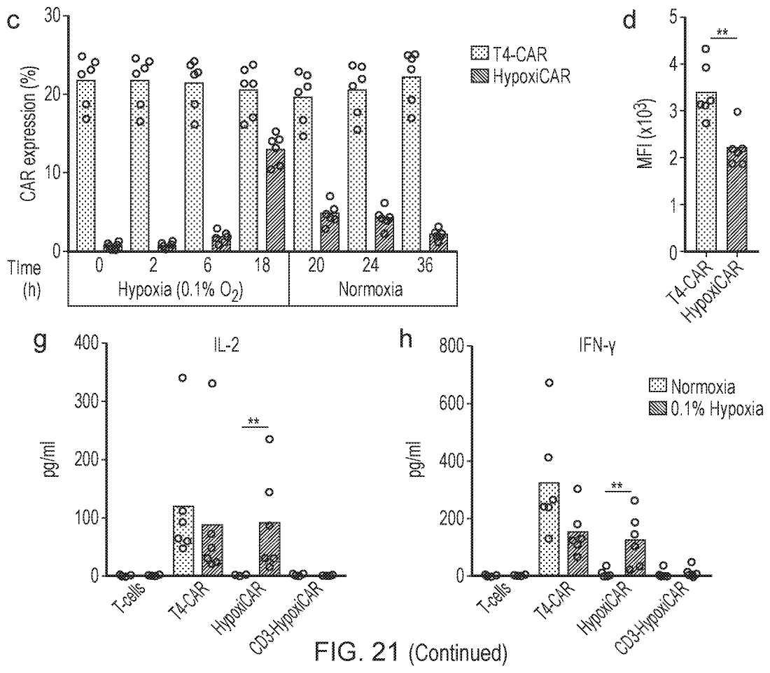

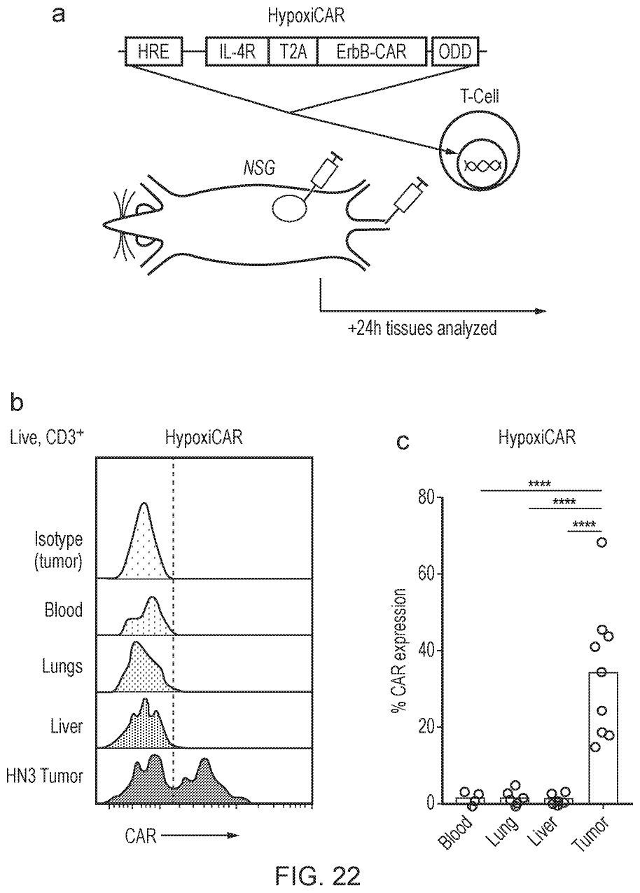

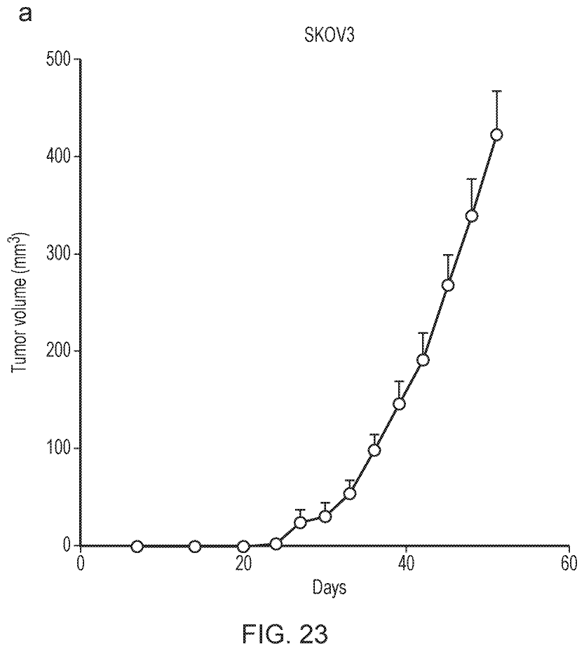

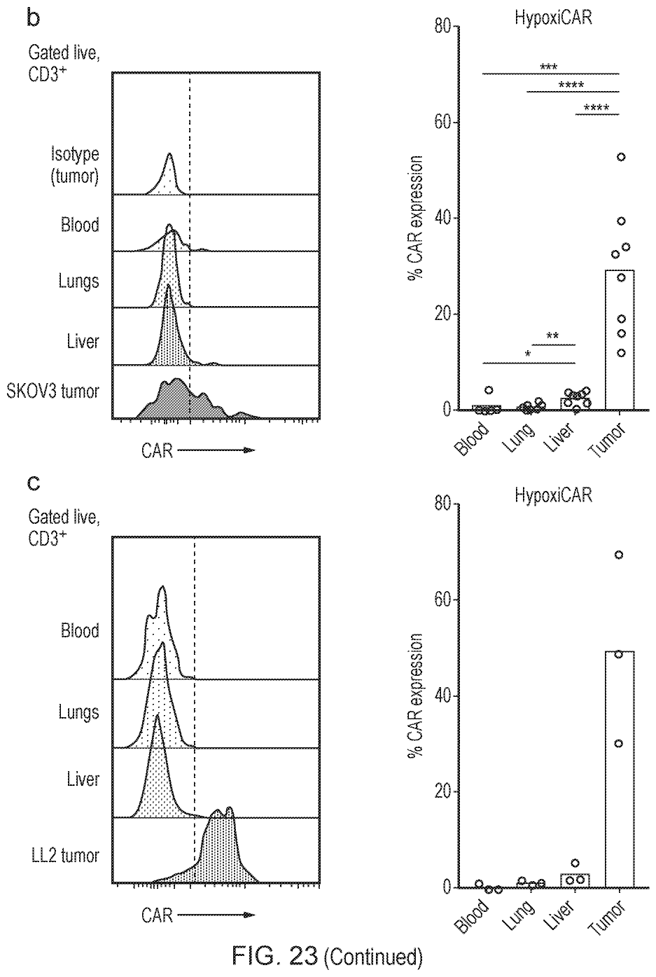

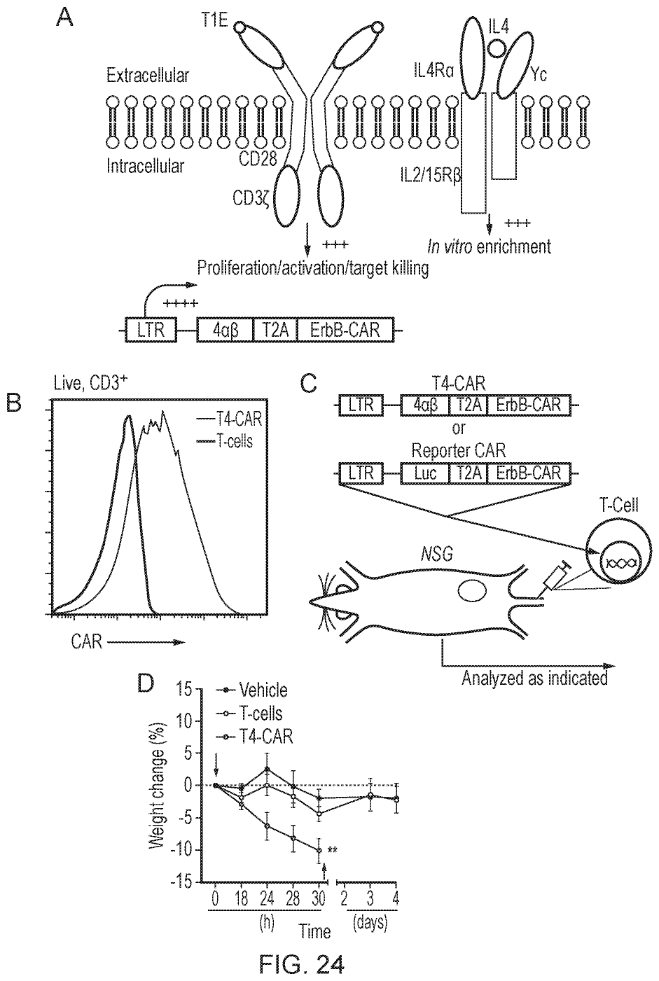

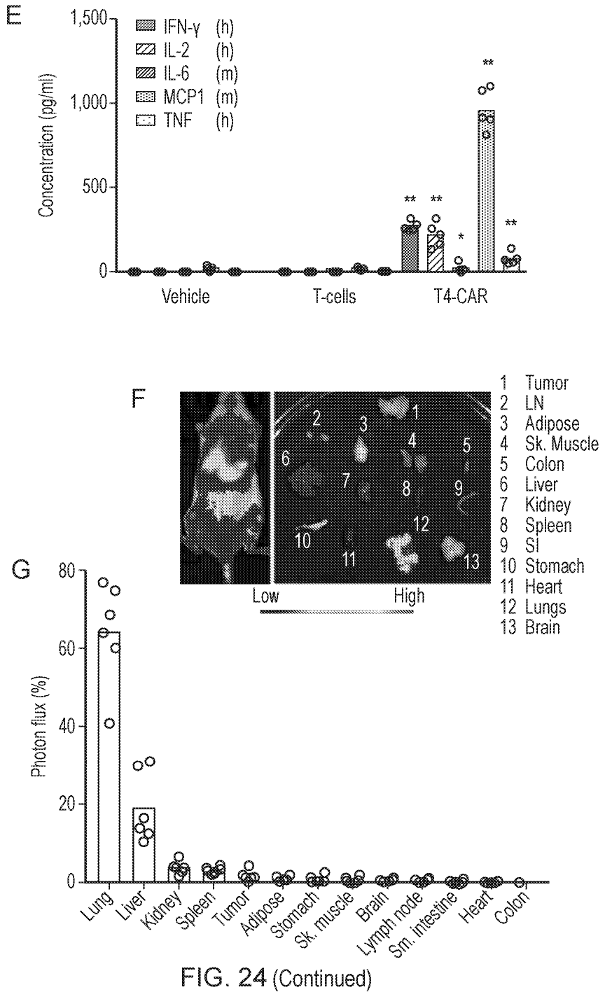

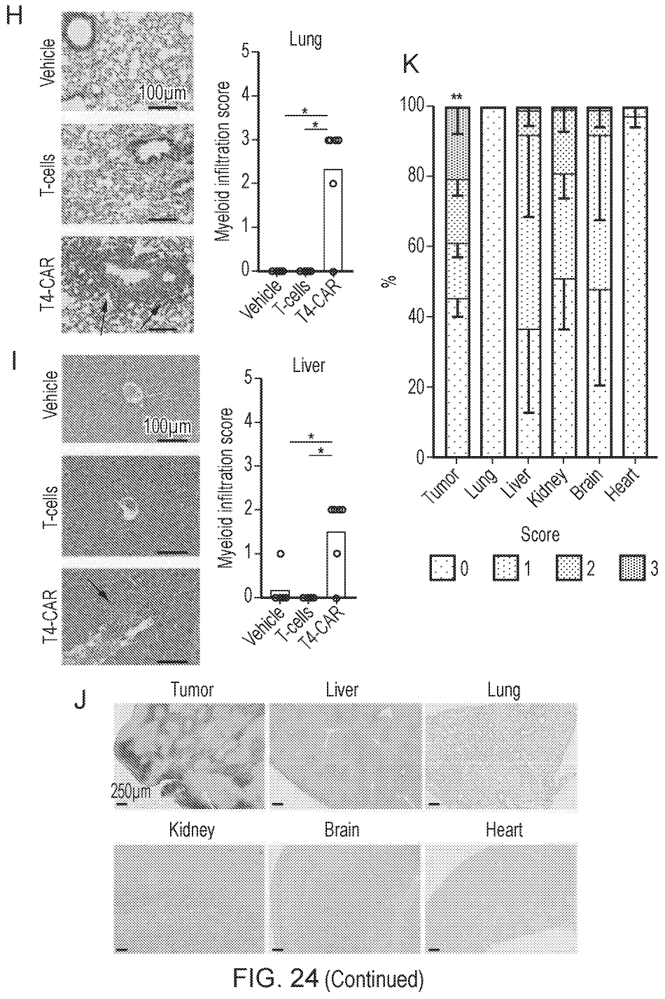

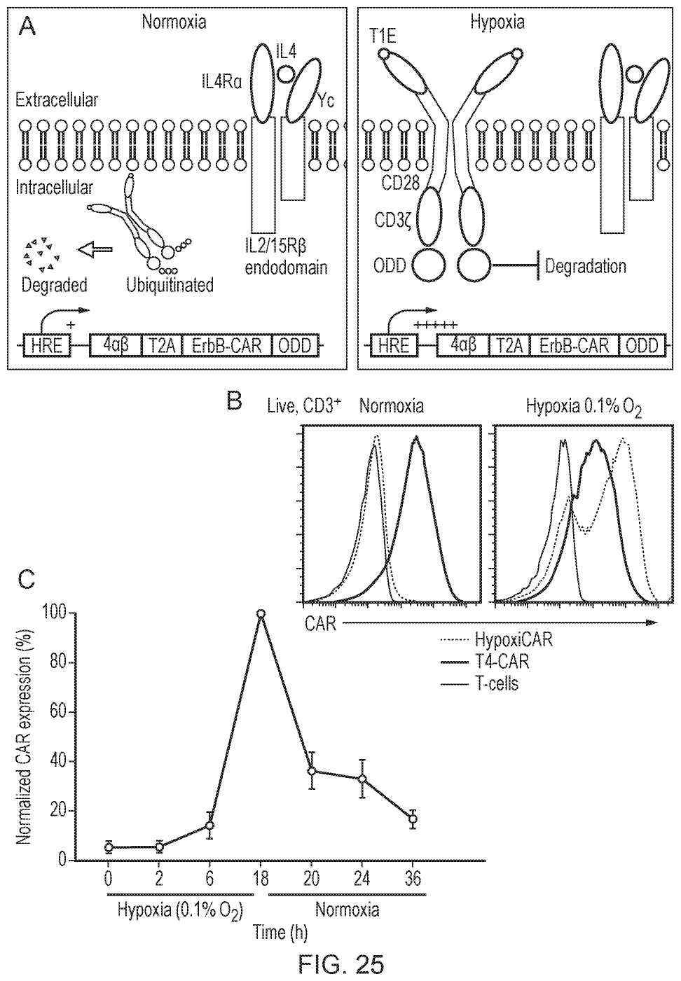

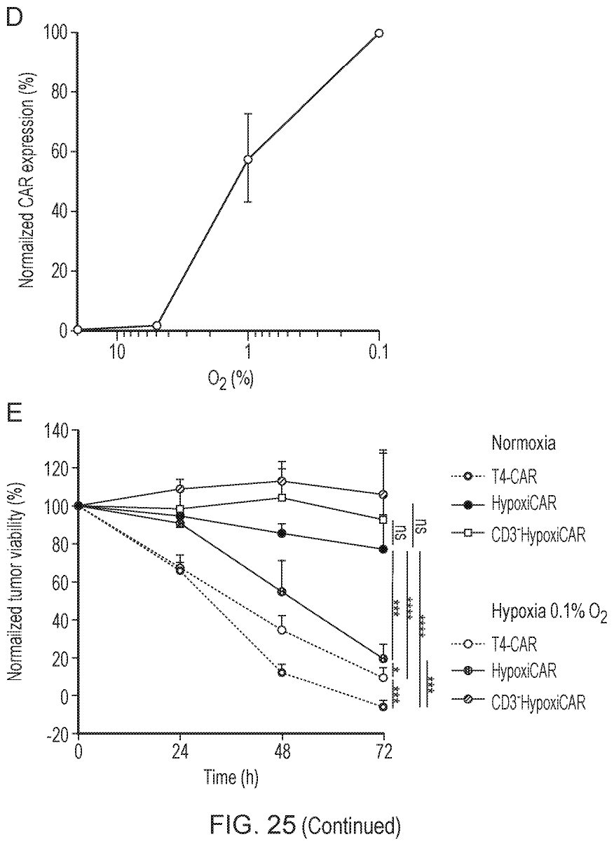

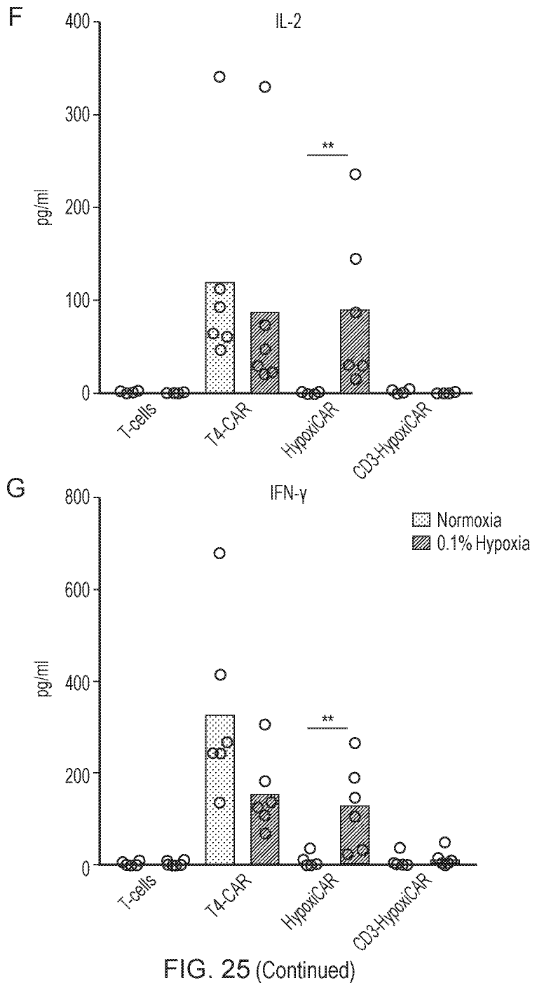

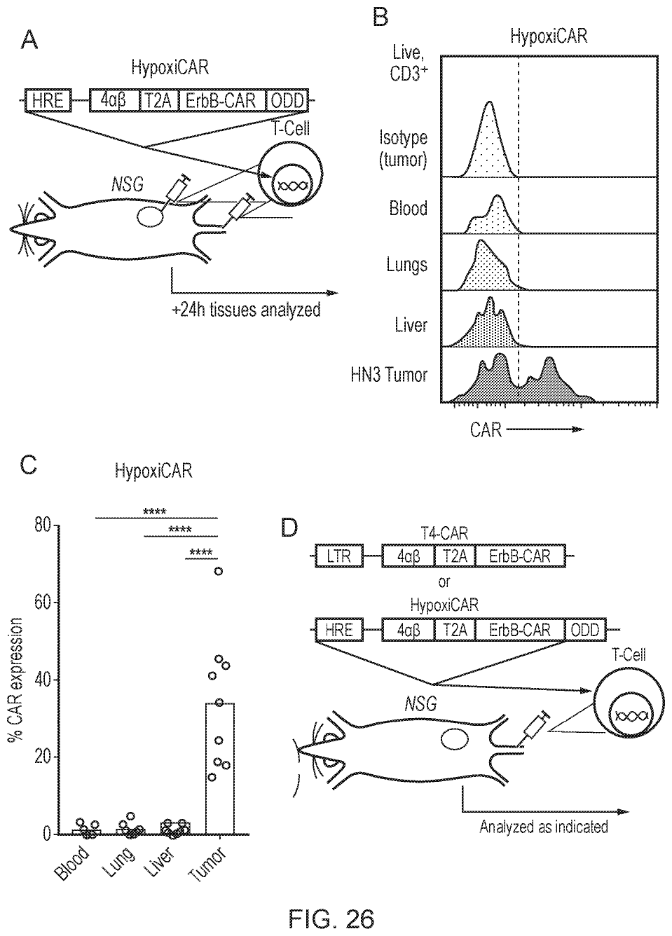

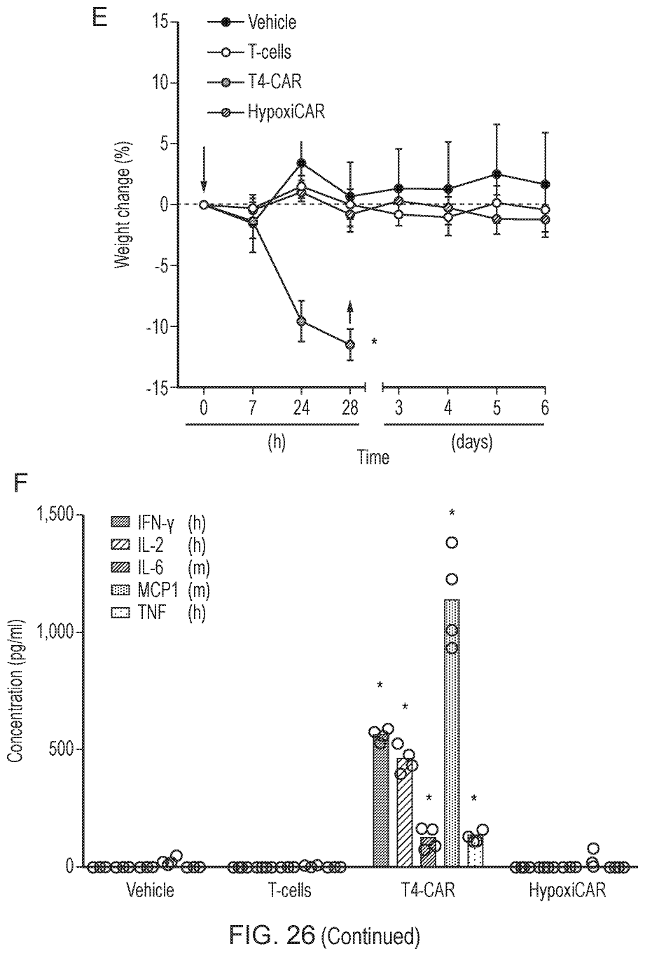

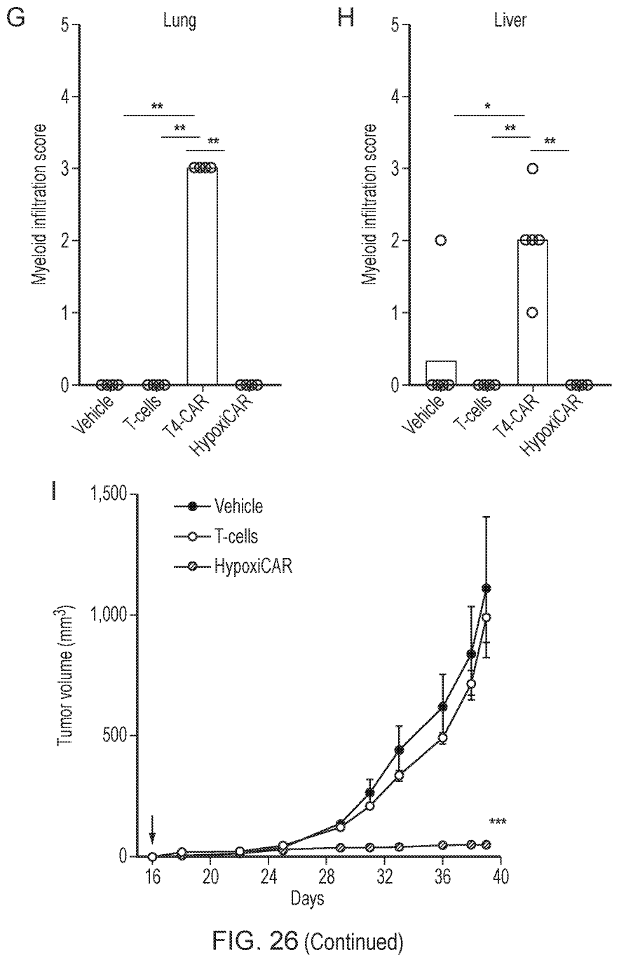

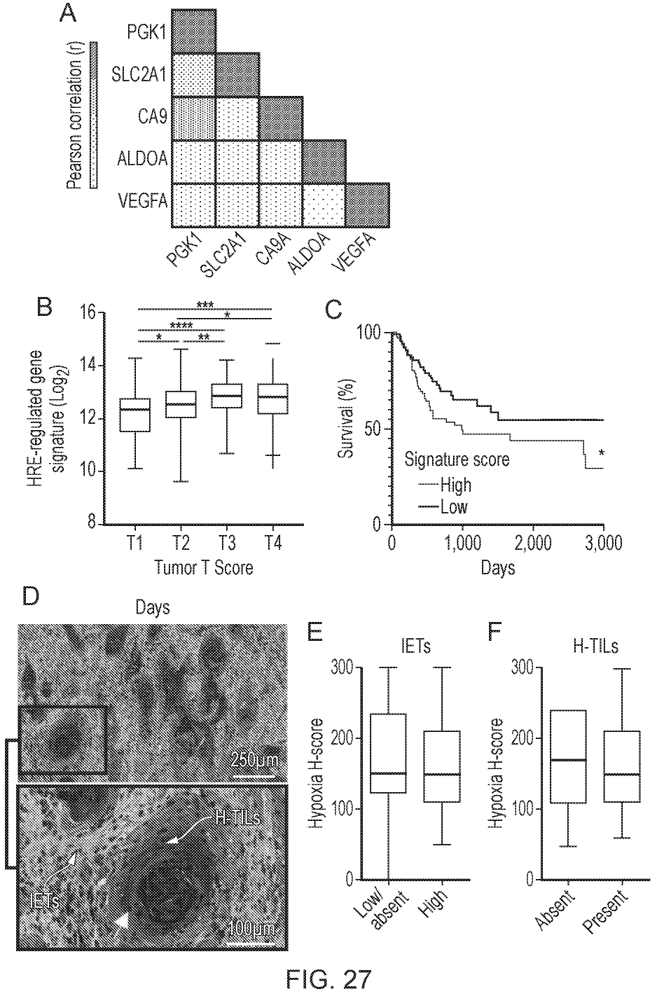

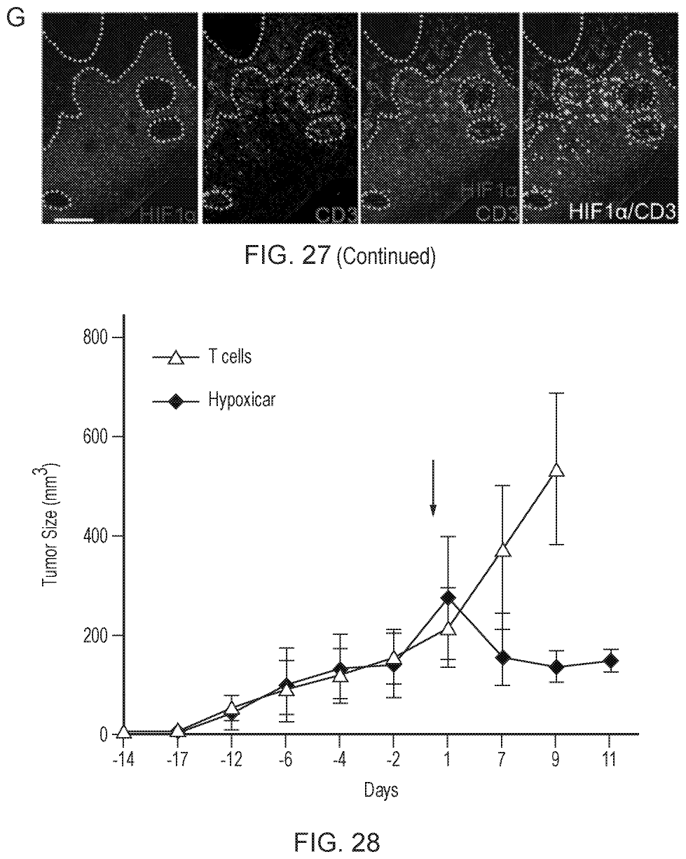

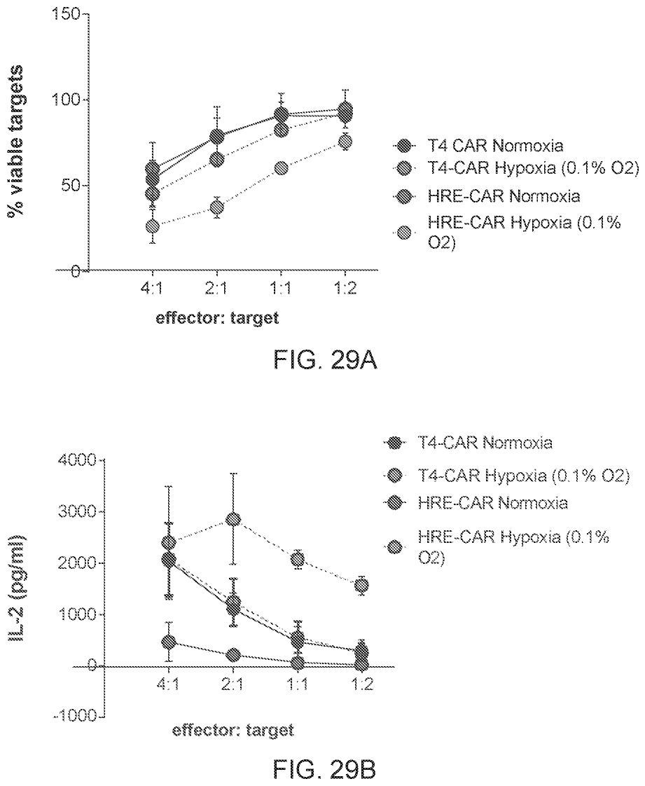

We utilised a pan-ErbB CAR T1E28z which has specificity towards 8/10 of the possible ErbB homo- and hetero-dimers in both mice and humans. We modified the CAR construct to concurrently co-express a reporter Click Beetle luciferase (Luc) to permit in vivo tracking once transduced into T-cells. ErbB-CAR/Luc T-cells were i.v. infused into immunocompromised NSG mice bearing subcutaneous SKOV3 ovarian cancer xenografts. The bio-distribution of the CAR T-cells was analysed 4 days post infusion. At this early time point, the majority of cells were seen to reside in the lungs and liver, while there was minimal uptake in the tumour (FIG. 19b). Profiling of organs for ErbB1-4 mRNA expression confirmed that all receptors from the family were expressed across all vital organs, including the lungs and liver where CAR T-cells were observed to accumulate post infusion.

As hypoxia differentiates the tumour microenvironment from healthy tissues, we sought to exploit this to create a hypoxia-sensing T4-CAR. T4 is a next generation anti-ErbB CAR co-expressed with a chimeric IL-4 receptor delivering an intracellular IL-2/IL-15 signal upon binding of IL-4 to the extracellular domain, thereby providing a means to selectively enrich CAR T-cells during ex vivo expansion without affecting the CAR-dependent killing capacity of the T-cells. We engineered the anti-ErbB CAR to contain a C-terminal 203 amino acid ODD and modified the CAR promoter in the long terminal repeat to contain a series of 9 HREs, rendering the CAR selectively responsive to hypoxia when transduced into T-cells (Schematic FIG. 21). In vitro, this CAR, named ‘HypoxiCAR’, demonstrated stringent hypoxia-specific surface CAR expression in both CD4⁺ and CD8⁺ T-cell populations (FIG. 21b).

CAR expression was highly dynamic and represented a switch that could be turned ‘on’ and ‘off’ in an O₂-dependent manner (FIG. 21c). The HRE proved to be a robust promoter as, in hypoxic conditions, only slightly less total cell surface CAR expression was observed compared to the parental T4-CAR, despite equivalent transduction efficiency and equal CD4/CD8⁺ T-cell ratio (FIG. 21d). HypoxiCAR demonstrated a favourable sensitivity of response to environmental O₂, where CAR expression was absent at O₂ concentrations found in healthy organs (≥5%) but detectable at O₂ levels seen in the tumour microenvironment (≤1%). Moreover, CAR expression positively correlated with the severity of hypoxia (FIG. 21e).

Having validated HypoxiCAR's ability to sense hypoxia, we sought to investigate its ability to elicit hypoxia-dependent killing of target cells. For this, SKOV3 ovarian cancer cells were used which express ErbB1-4. Cells were seeded onto culture plates and co-incubated with T4-CAR or HypoxiCAR under normoxic (20% O₂) and hypoxic (0.1% O₂) conditions. Despite equivalent transduction efficiencies, HypoxiCAR displayed efficient hypoxia-dependent killing of the SKOV3 cells with no significant killing under normoxic conditions. Target-cell killing was CAR-mediated as when HypoxiCAR's intracellular tail was truncated to prevent signalling (CD3⁻), killing was abrogated (FIG. 210. We also assessed the secretion of both IL-2 (FIG. 21g) and IFNγ (FIG. 21g-h) in these co-cultures, two cytokines which play important role in the T-cell response. Cytokine production by hypoxiCAR T-cells was also stringently regulated such that detectable levels were only found under hypoxic conditions.

To translate these observations in vivo, we evaluated whether HypoxiCAR could circumvent off-tumour toxicity of ErbB-CAR T-cells. This is a major hurdle that precludes their systemic administration in the clinic. To evaluate this technology in the tumour setting, HypoxiCAR T-cells were injected concurrently i.v. and i.t. in HN3 tumour-bearing NSG mice. By this means, we achieved a rapid accumulation of these cells in tumour and vital organs for ex vivo investigation (FIG. 22A). Four days after HypoxiCAR infusion, tissues were harvested, enzyme-digested and T-cells were assessed for CAR expression using flow cytometry. HypoxiCAR achieved tumour-selectivity of expression and only presented surface CAR molecules within the hypoxic tumour microenvironment, with an absence of CAR expression when T-cells were located in the blood, lungs, and liver (FIG. 22B-C). This observation was not model specific as it was also observed in NSG mice bearing SKOV3 tumours and Rag2^{-/-} mice bearing murine Lewis lung carcinoma (LL2) tumours.

The results show a stringent hypoxia-sensing CAR T-cell approach which achieves selective expression of a panErbB-targeted CAR within a solid tumour, a microenvironment characterized by an inadequate oxygen supply. Despite widespread expression of ErbB receptors in healthy organs, the approach provides anti-tumour efficacy without off-tumour toxicity in murine xenograft models. This dynamic oxygen-sensing safety switch potentially facilitates unlimited expansion of the CAR T-cell target repertoire for treating solid malignancies.

Identifying approaches to circumvent off-tumour toxicity has the potential to unlock an entirely new repertoire of CAR antigen targets for carcinomas, which are currently limited.

To investigate this issue, we utilized a 2^{nd} generation pan-anti-ErbB CAR T1E28z which has specificity towards 8/10 of the possible ErbB receptor homo- and hetero-dimers and crosses the species barrier binding both mice and human receptors equivalently. This CAR is currently undergoing Phase I evaluation by intra-tumoural (i.t.) delivery in patients with SCCHN. The CAR is co-expressed with a chimeric cytokine receptor (4αβ) which delivers an intracellular IL-2/IL-15 signal upon binding of IL-4 to the extracellular domain (FIGS. 24 A and B), providing a means to selectively enrich CAR T-cells during ex vivo expansion, but however does not affect the CAR-dependent killing capacity of the T-cells. This combination is referred to as T4-immunotherapy. Although i.t. delivery of T4-CAR T-cells has proven safe in man, i.v. infusion is desirable as this permits these cells to home to both the primary tumour and metastasis. I.v. infusion of human T4-CAR T-cells into immunocompromised NSG mice bearing HN3 tumours (FIG. 24C) which express ErbB1-4 resulted in lethal toxicity, evident by a rapid loss of weight in these animals (FIG. 24D). As observed clinically, analysis of the blood of these mice revealed evidence of an increase in pro-inflammatory cytokines (FIG. 24E). In an attempt to resolve the biodistribution of CAR T-cells, we modified the CAR construct to concurrently express a luciferase (Luc) reporter to permit in vivo tracking of transduced T-cells (FIG. 24F). Imaging analysis four days post i.v. infusion of a sub-lethal dose of reporter human CAR T-cells revealed that the majority had accumulated in the lungs and liver, while only a minority were present in the tumour despite the expression of ErbB1-4 (CAR targets) on these cells (FIG. 24G). The accumulation in the liver and lung was not an artefact of the xenograft system as, when murine T-cells were transduced to express the same reporter CAR and infused i.v. into Rag2^{-/-} mice (FIG. 24C), they accumulated in the same tissues and in the spleen (Fig. S3). Notably, murine T-cell accumulation in the liver, but not the lung, was CAR-dependent as T-cells expressing the Luc reporter alone were significantly less prevalent at this location. The CAR-independent T-cell accumulation in the lung was likely due to an integrin-dependent interaction. Profiling of ErbB1-4 mRNA expression confirmed that all four receptors were expressed in all vital organs, including the lungs and liver. To investigate for direct evidence of T4-CAR T-cell-mediated tissue damage, a sub-lethal dose of human T4-CAR T-cells was infused i.v. into NSG mice and a pathohistological examination using haematoxylin and eosin (H&E) stained tissue sections of the liver and lung was conducted after 5 days. This analysis revealed the presence of myeloid cell infiltrates in the lungs and liver (FIGS. 24H and I), representing a surrogate marker of CAR-mediated inflammation. The infiltrate was observed both in a perivascular distribution and scattered throughout the parenchyma, consisting of both neutrophils (polymorphonuclear cells) and large mononuclear cells with abundant cytoplasm, likely to be macrophages. Hepatocyte necrosis/apoptosis was also seen in some animals. T4-CAR T-cells accumulated in the kidney at a lower level (FIG. 24G) with no significant evidence of inflammation in this tissue. These data indicate that the liver and lung represent the two key organs for off-tumour CAR T-cell activation.

Hypoxia is a characteristic of most solid tumours. The proliferative and high metabolic demands of the tumour cells, alongside inefficient tumour vasculature, result in a state of inadequate oxygen supply (<2% O₂) compared to that of healthy organs/tissues (5-10% O₂) (FIGS. 24 J and K). As hypoxia differentiates the tumour microenvironment from that of healthy, normoxic tissue, it represents a desirable marker for the induction of CAR T-cell expression (FIGS. 24 J and K). To create a stringent hypoxia-regulated CAR expression system, we developed a dual-oxygen sensing approach for the T4-CAR (FIG. 25A). This was achieved by appending a C-terminal 203 amino acid ODD onto the anti-ErbB CAR while concurrently modifying the CAR promoter in the long terminal repeat (LTR) enhancer region to contain a series of 9 consecutive HREs, rendering CAR expression selectively responsive to hypoxia. In vitro, this CAR, named ‘HypoxiCAR’, demonstrated stringent hypoxia-specific presentation of the CAR molecules on the cell surface of human T-cells (FIG. 25B). We demonstrated that the dual-oxygen sensing system proved superior to variants in which either the 9 HRE cassette or ODD were used alone. In both cases, these alternative approaches displayed leakiness of CAR expression under conditions of normoxia, permitting tumour cell killing under normoxic conditions. HypoxiCAR's expression of the CAR was also highly dynamic and represented a switch that could be turned both ‘on’ and ‘off’ in an O₂-dependent manner (FIG. 25C). In further in vitro characterization, exquisite O₂ sensitivity of HypoxiCAR was confirmed as CAR expression was absent under O₂ concentrations consistent with healthy organs (5%) but became detectable on the cell surface at O₂ concentrations equivalent to those found in the tumour microenvironment (1%) (FIG. 25D). Tumour-infiltrated T-cells have been demonstrated to egress from the tumour microenvironment, highlighting a potential safety concern if hypoxia-experienced HypoxiCAR T-cells expressing CAR were to re-enter healthy normoxic tissue. However, as cytolytic T-cell mediated killing of a target cell may take up to 6 hours, within which time in normoxia it might be expected that approximately 62±8% of HypoxiCAR's surface CAR may have already degraded (FIG. 2C), any off-tumour killing by egressed HypoxiCAR T-cells would be expected to be limited. Moreover, once HypoxiCAR has expressed sufficient CAR to kill a target, cell egress would be limited as has been demonstrated that CD8⁺ T-cell migration ceases in regions where it encounters a tumour cell expressing its cognate antigen.

Having validated HypoxiCAR's ability to sense hypoxia, we sought to investigate its ability to elicit hypoxia-dependent killing of tumour target cells. SKOV3 ovarian cancer cells were seeded onto culture plates and co-incubated with T4-CAR or HypoxiCAR under normoxic and hypoxic (0.1% O₂) conditions. Despite equivalent transduction efficiencies and CD4⁺:CD8⁺ T-cells ratios, HypoxiCAR T-cells displayed efficient hypoxia-dependent killing of the SKOV3 cells, almost equivalent to T4-CAR T-cells, with no significant killing observed under normoxic conditions (FIG. 25E). Target-cell destruction was strictly CAR-dependent as when the intracellular tail of HypoxiCAR was truncated to prevent CD3□ signalling, killing was abrogated (FIG. 25E). In addition, HypoxiCAR-provided stringent hypoxia-restricted T-cell secretion of both IL-2 (FIG. 25F) and IFNγ (FIG. 25G), two cytokines which play an important role in the T-cell response.

To evaluate whether HypoxiCAR could provide tumour-restricted CAR expression in vivo, human HypoxiCAR T-cells were injected concurrently i.v. and i.t. in NSG mice bearing HN3 tumours. These tumours had an approximate volume of 500 mm³ (FIG. 26A), in which the presence of hypoxia was confirmed (FIG. 24J,K). Four days after HypoxiCAR T-cell infusion, tissues were harvested, enzyme-digested and T-cells were assessed for CAR expression using flow cytometry. As predicted by the in vitro analyses (FIG. 25), HypoxiCAR T-cells did not express detectable cell surface CAR molecules when recovered from the blood, lungs, or liver of the mice post infusion, but they did express CAR molecules on the cell surface within the hypoxic tumour microenvironment (FIG. 26B,C). This finding was not model specific as similar observations were made in both NSG mice bearing SKOV3 tumours and in Rag2^{-/-} mice bearing murine Lewis lung carcinoma (LL2) tumours. To establish if the ‘Hypoxi’ construct elements would be active across different stages of tumour growth, a Hypoxi-luciferase reporter was developed in which the HRE promoter was used to drive expression of a luciferase-ODD. This reporter was stably transduced directly into the SKOV3 and HN3 cell lines. Luciferase-ODD, despite not being detectable in tumour cells under normoxic conditions, was detected in vivo at all stages of tumour growth, even prior to the tumour becoming palpable, in both SKOV3 and HN3 tumours. This suggested that HypoxiCAR T-cells might be active even against early stage tumours. To test this, HypoxiCAR T-cells were infused into mice at day 16 post injection of HN3 tumour cells, just prior to the tumours becoming palpable. In keeping with the absence of CAR expression on the T-cells in normoxic tissues, HypoxiCAR also circumvented the treatment-limiting toxicity seen using following i.v. infusion of high-dose T4-CAR T-cells. Indeed, mice infused i.v. with human HypoxiCAR T-cells displayed no acute drop in weight post infusion (FIG. 26D,E), no evidence of pro-inflammatory cytokines in the systemic circulation (FIG. 26F), nor were there any signs of tissue damage in the lung, liver or kidney (FIG. 26G,H). Importantly, while mice infused i.v. with human T4-CAR T-cells all reached their humane endpoints at 28 h (FIG. 26E), the HypoxiCAR T-cell infused mice showed no signs of off-tumour toxicity and prevented tumour growth (FIG. 26I). As such, HypoxiCAR overcomes a major hurdle that currently precludes the systemic administration of CAR T-cells targeting antigens that are expressed in normal tissues throughout the body.

Hypoxia has been extensively studied in SCCHN. To assess which patients might be most appropriate for HypoxiCAR T-cell immunotherapy, we firstly generated an HRE-regulated gene signature using patient tumour transcriptomic data. Known HRE-regulated genes were analyzed for co-expression, and a refined signature utilizing the genes PGK1, SLC2A1, CA9, ALDOA and VEGFA was chosen as we observed a significant positive correlation between these genes (FIG. 27A). There was no difference in expression of this signature across the different SCCHN subtypes (hypopharynx, larynx, oral cavity, and oropharynx). However, expression of this 5-gene signature, significantly increased with tumour size (T-score; FIG. 27B) and was also prognostic of poorer survival in stage 3 and 4 HNSCC patients (FIG. 27C). Utilizing an HRE-regulated gene signatures to predict hypoxia from biopsy material could provide a simple means to assess those patients which might respond best to HypoxiCAR therapy.

Immunohistochemistry staining of SCCHN tumour sections for stabilized HIF1α, the master transcription factor for HypoxiCAR's CAR expression, revealed large regions of the tumours where HIF1α had become stabilized (FIG. 27D). Although several factors can stabilize HIF1α, hypoxia represents the most probable explanation for this observation. Heterogeneity in both HIF1α stabilization and intra-tumoural T-cell infiltration was seen between patients. Encouragingly however, those tumours with the highest prevalence and/or intensity of HIF1α stabilization did not exclude T-cells from entering the intra-epithelial space nor from entering HIF1α stabilized regions of the tumour (FIG. 27E,F). Using immunofluorescence, we also demonstrated that CD3⁺ T-cells infiltrating HIF1α stabilized tumour regions also stabilized HIF1α themselves, suggesting that in these environments HypoxiCAR T-cells would become activated (FIG. 27G). These observations suggest that HypoxiCAR could find clinical application in hypoxic tumour types such as SCCHN, where gene expression (FIG. 27A-C), staining of biopsy samples for HIF1α/CD3 (FIG. 27D-G) and imaging techniques such as PET/CT using a hypoxia-radiotracer such as ⁶⁴Cu-ATSM might provide biomarkers to confirm the presence of a hypoxic tumour microenvironment and guide patient selection.

Approaches to improve tumour-specificity of CAR T-cells have been developed, such as T-cell receptor-mimetic CARs with specificity for HLA-presented antigens, combined targeting of tumour antigens, or tuning of CAR affinity to preferentially target high density antigens. This study demonstrates an alternative approach to achieve cancer-selective immunotherapy, exploiting one of the most innate characteristics of the tumour microenvironment. The ‘dual hypoxia-sensing’ system described here achieves compelling anti-tumour efficacy while abrogating off-tumour toxicity of a CAR that recognizes multiple targets in normal tissues. The hypoxia-sensing HRE module and the ODD appended onto the CAR act synergistically to provide stringent hypoxia-specific target killing (FIG. 25E). This approach restricts both transcription (HRE) and stability (ODD) of the CAR under conditions of normoxia and, when these two systems are utilized concurrently, they overcame the leakiness observed when either system was used alone.

The hypoxic tumour microenvironment is not conducive to efficient immune reactions. Hypoxia can activate immune-suppressive programmes in stromal cells such as macrophages, regulate the expression of immune checkpoint molecules and promote a more aggressive tumour cell phenotype. However, encouragingly we found that hypoxia did not negatively affect T-cell effector function directly in vitro (FIG. 25E-G), which is in agreement with that observed by others. HypoxiCAR T-cells also were able to prevent the growth of hypoxic tumours (FIG. 26I) suggesting that, in the in vivo models tested, the tumour microenvironment was not a complete barrier to HypoxiCAR's ability to deliver in vivo anti-tumour therapeutic efficacy. There is also the potential in the future to combine HypoxiCAR T-cell therapy with microenvironment modifying agents, such as immune checkpoint inhibitors, which may further improve the ability of these cells to target the tumour. Furthermore, as T-cells are not excluded from HIF1α stabilized regions of human tumours (FIG. 27D-F) it is likely that HypoxiCAR T-cells should be able to access the appropriate microenvironments to activate CAR expression. Although we did not observe evidence of treatment-limiting toxicity in mice infused with high dose HypoxiCAR T-cells (FIGS. 26E and I), there are microenvironments in healthy tissues such as the intestinal mucosa where ‘physiologic hypoxia’ has been observed. Such tissues might represent sites where off-tumour activation of HypoxiCAR

T-cells could take place. As such, a suicide switch could be incorporated into HypoxiCAR to provide an additional level of safety for the most pervasive CARs. Although the ‘HypoxiCAR’ dual oxygen sensing system was exemplified using a pan-ErbB-targeted CAR, the broadly applicable strategy may be used to overcome the paucity of safe targets available for the treatment of solid malignancies.

1. A nucleic acid molecule comprising:
a. a polynucleotide encoding a Chimeric Antigen Receptor (CAR), wherein the CAR comprises:
(i) one or more Oxygen-Dependent Degradation Domains (ODD); and
(ii) at least one polypeptide with anti-tumour properties; and
b. a hypoxia-responsive regulatory nucleic acid,
wherein said CAR-encoding polynucleotide is operably linked to said hypoxia-responsive regulatory nucleic acid.

2. The nucleic acid molecule of claim 1, wherein said hypoxia-responsive regulatory nucleic acid comprises a plurality of hypoxia-responsive elements (HREs), wherein each individual HRE of said plurality of HREs independently comprises (i) an HIF binding site (HBS): 5′-(A/G)CGT(G/C)-3′ (SEQ ID NO: 1); and optionally (ii) an HIF ancillary site (HAS): 5′-CA(C/G)(G/A)(T/C/G)-3′ (SEQ ID NO: 2); or (iii) an HNF-4 site: 5′-TGACCT-3′ (SEQ ID NO: 3).

3. The nucleic acid molecule of claim 2, wherein said HBS and HAS if present are separated by a linker, optionally wherein said linker is at least 6 nucleotides in length.

4. The nucleic acid molecule of claim 2, wherein said plurality of HREs comprises at least one or a plurality of sequences selected from SEQ ID NOs 5-17 or sequences having at least 70%, 75%, 80%, 85%, 90%, 95% or more sequence identity to any of SEQ ID NOs 5-17.

5. The nucleic acid molecule of claim 2, wherein said plurality is at least two, three, four, five, six, seven, eight, nine, ten, eleven, twelve, thirteen, fourteen, fifteen, sixteen, seventeen, eighteen, nineteen, twenty or more individual HREs, which may be sequentially positioned or which may be spatially separate.

6. (canceled)

7. The nucleic acid molecule of claim 2, wherein said hypoxia-responsive regulatory nucleic acid comprises a sequence of SEQ ID NOs 18, 19, 20, 21, 22, 23, 24, 25, 26 or 27 or functional fragment thereof or homologues thereof.

8. The nucleic acid molecule of claim 2, wherein said hypoxia-responsive regulatory nucleic acid comprises a sequence of SEQ ID NO 19 or 26 or a homologue thereof having at least 70%, 75%, 80%, 85%, 90%, 95% or more sequence identity thereto.

9. The nucleic acid molecule of claim 2, wherein the hypoxia responsive regulatory nucleic acid is comprised in a retroviral or lentiviral vector, optionally an SFG retroviral vector.

10. The nucleic acid molecule of claim 9, wherein the retroviral or lentiviral vector comprises an enhancer region, wherein the enhancer region comprises a plurality of HREs, optionally wherein the plurality is nine HREs which may be sequentially positioned or which may be spatially separate.

11. (canceled)

12. The nucleic acid molecule of claim 2, wherein said HREs are derived from any one or more of the following oxygen-responsive genes or from orthologues or paralogues thereof: erythropoietin (EPO), vascular endothelial growth factor (VEGF), phosphoglycerate kinase (PGK), glucose transporters (e.g. Glut-1), lactate dehydrogenase (LDH), aldolase (ALD), Enolase (e.g. ENO3), glyceraldehyde-3-phosphate dehydrogenase (GAPDH), nitric oxide synthetase (NOS), Heme oxygenase, muscle glycolytic enzyme pyruvate kinase (PKM), endothelin-1 (ET 1).

13. The nucleic acid molecule of claim 1, wherein said ODD has the sequence of SEQ ID NO: 28: X¹X²LEMLAPYIXMDDDX³X⁴X⁵, where “X¹⁻⁵” can be any amino acid residue, optionally wherein X¹ is “L” or any conservative substitution; X² is “D” or any conservative substitution, X³ is “F” or any conservative substitution, X⁴ is “Q” or any conservative substitution, X⁵ is “L” or any conservative substitution.

14. The nucleic acid molecule of claim 1, wherein said ODD has the sequence of SEQ ID NO: 29, 30 or 31 or homologue thereof having at least 70%, 75%, 80%, 85%, 90%, 95% or more sequence identity thereto and comprising SEQ ID NO: 28 or the sequence of SEQ ID NO: 5 or variant thereof having at least 70%, 75%, 80%, 85%, 90%, 95% or more sequence identity to SEQ ID NO: 5, wherein said variant comprises SEQ ID NO: 4.

15. (canceled)

16. The nucleic acid molecule of claim 1, wherein said polypeptide with an anti-tumour property comprises:
a. an extracellular antigen-specific targeting region, or
b. a protein for delivery to a tumour, selected from immune stimulating antibodies; surface or intracellular receptors that confer cell activation and tumour-killing capability; T-cell Receptor (TCR); immunomodulatory cytokines (for example, IL-12, IL-15), decoy antibodies (for example, PD axis-interacting antibodies), and a protein that alters host cell function (for example, Lck, TCR zeta chain, ZAP70).

17. (canceled)

18. (canceled)

19. The nucleic acid molecule of claim 1, wherein hypoxia is a condition with O₂ concentration below 5%, preferably below 3%, or reduced O₂ availability relative to O₂ availability or partial pressure of the corresponding non-cancerous organ, tissue or cells.

20. (canceled)

21. The nucleic acid molecule of claim 1, wherein said CAR is selected from a first, second, third, fourth generation CAR, a split CAR design, and armoured CAR.

22. The nucleic acid molecule of claim 1, wherein said CAR has specificity towards the ErbB family of receptors.

23. An immunoresponsive cell comprising said nucleic acid molecule of claim 1.

24. (canceled)

25. (canceled)

26. A method for the preparation of a modified immunoresponsive cell, comprising:
a isolating lymphoid or myeloid-derived cells from a subject;
b. modifying said cells to introduce the nucleic acid molecule of claim 1;
c. expanding said modified cells ex-vivo; and
d. obtaining expanded cells capable of expressing said nucleic acid molecule under conditions of hypoxia.

27. (canceled)

28. (canceled)

29. A method for treatment of haematological or solid cancer, comprising administering the immunoresponsive cell of claim 23 to a patient in need thereof.

30. (canceled)

31. (canceled)

32. (canceled)

33. A pharmaceutical composition comprising the immunoresponsive cell of claim 23.

34. (canceled)

35. (canceled)

36. (canceled)

37. (canceled)

38. (canceled)

39. (canceled)

40. (canceled)

41. The method of treatment of claim 29, further comprising a preceding step of:
a monitoring co-expression of at least two, three, four or all five the following genes: PGK1, SLC2A1, CA9, ALDOA and VEGFA, wherein co-expression of said genes in said subject is indicative of the subject's suitability for treatment,
b. immunohistochemical staining of a tumour biopsy from the subject and assessing HIF stabilisation in the tumour or stoma, or
c. monitoring T-cell infiltration (and/or of other immunoresponsive cells) to HIF stabilised regions of the tumour, wherein infiltration of the immunoresponsive cells to HIF stabilised regions of the tumour is indicative of a subject's suitability for treatment.
